(19) United States
(12) Patent Application Publication
(10) Pub. No.: US 2009/0062223 A1
(43) Pub. Date: Mar. 5, 2009

(54) AMINO TRICYCLIC-NUCLEOSIDE COMPOUNDS, COMPOSITIONS, AND METHODS OF USE

(75) Inventors: Jesse Daniel Keicher, San Carlos, CA (US); Christopher Don Roberts, Belmont, CA (US); Vivek Kumar Rajwanshi, Cupertino, CA (US); Ronald Conrad Griffith, Escondido, CA (US); Xiaoling Zheng, Fremont, CA (US); Sebastian Johannes Reinhard Liehr, Palo Alto, CA (US); Marija Prhavc, Encinitas, CA (US); Choung U. Kim, San Carlos, CA (US); Adrian S. Ray, Redwood City, CA (US)

Correspondence Address: Genelabs Technologies, Inc.;c/o Foley & Lardner LLP, 975 Page Mill Road, Palo Alto, CA, 94304-1013, US

(73) Assignee: GENELABS TECHNOLOGIES, INC.

(21) Appl. No.: 12/201,692

(22) Filed: Aug. 29, 2008

Related U.S. Application Data
(60) Provisional application No. 60/969,581, filed on Aug. 31, 2007.

(51) Int. Cl. A61K 31/7064; C07H 19/23; A61P 31/14

(52) U.S. Cl. 514/43; 536/27.1

(57) ABSTRACT

Provided are compounds of Formula (I) or a pharmaceutically acceptable salt or solvate thereof. The compounds and compositions are useful for treating viral infections caused by the Flaviviridae family of viruses.

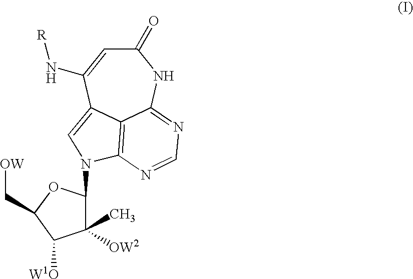

### CROSS-REFERENCE TO RELATED APPLICATION

This application claims the benefit under 35 USC 119(e) to co-pending U.S. Provisional Application No. 60/969,581, filed 31 Aug. 2007, which is incorporated into this application by reference in its entirety.

### BACKGROUND OF THE INVENTION

### Field of the Invention

Compounds and compositions, methods for their preparation, and methods for their use in treating viral infections in patients mediated, at least in part, by a virus in the Flaviviridae family of viruses are disclosed.

### REFERENCES

The following publications are cited in this application as superscript numbers:
1. Szabo, E. et al., Pathol. Oncol. Res. 2003, 9:215-221.
2. Hoofnagle J. H., Hepatology 1997, 26:15 S-20S.
3. Thomson B. J. and Finch R. G., Clin Microbial Infect. 2005, 11:86-94.
4. Moriishi K. and Matsuura Y., Antivir. Chem. Chemother. 2003, 14:285-297.
5. Fried, M. W., et al. N. Engl. J. Med 2002, 347:975-982.
6. Ni, Z. J. and Wagman, A. S. Curr. Opin. Drug Discov. Devel 2004, 7, 446-459.
7. Beaulieu, P. L. and Tsantrizos, Y. S. Curr. Opin. Investig. Drugs 2004, 5, 838-850.
8. Griffith, R. C. et al., Ann. Rep. Med. Chem. 39, 223-237, 2004.
9. Watashi, K. et al., Molecular Cell, 19, 111-122, 2005
10. Horsmans, Y. et al., Hepatology, 42, 724-731, 2005

### STATE OF THE ART

Chronic infection with HCV is a major health problem associated with liver cirrhosis, hepatocellular carcinoma, and liver failure. An estimated 170 million chronic carriers worldwide are at risk of developing liver disease.^{1,2} In the United States alone 2.7 million are chronically infected with HCV, and the number of HCV-related deaths in 2000 was estimated between 8,000 and 10,000, a number that is expected to increase significantly over the next years. Infection by HCV is insidious in a high proportion of chronically infected (and infectious) carriers who may not experience clinical symptoms for many years. Liver cirrhosis can ultimately lead to liver failure. Liver failure resulting from chronic HCV infection is now recognized as a leading cause of liver transplantation.

HCV is a member of the Flaviviridae family of RNA viruses that affect animals and humans. The genome is a single ˜9.6-kilobase strand of RNA, and consists of one open reading frame that encodes for a polyprotein of ˜3000 amino acids flanked by untranslated regions at both 5′ and 3′ ends (5′- and 3′-UTR). The polyprotein serves as the precursor to at least 10 separate viral proteins critical for replication and assembly of progeny viral particles. The organization of structural and non-structural proteins in the HCV polyprotein is as follows: C-E1-E2-p7-NS2-NS3-NS4a-NS4b-NS5a-NS5b. Because the replicative cycle of HCV does not involve any DNA intermediate and the virus is not integrated into the host genome, HCV infection can theoretically be cured. While the pathology of HCV infection affects mainly the liver, the virus is found in other cell types in the body including peripheral blood lymphocytes.^{3,4}

At present, the standard treatment for chronic HCV is interferon alpha (IFN-alpha) in combination with ribavirin and this requires at least six (6) months of treatment. IFN-alpha belongs to a family of naturally occurring small proteins with characteristic biological effects such as antiviral, immunoregulatory, and antitumoral activities that are produced and secreted by most animal nucleated cells in response to several diseases, in particular viral infections. IFN-alpha is an important regulator of growth and differentiation affecting cellular communication and immunological control. Treatment of HCV with interferon has frequently been associated with adverse side effects such as fatigue, fever, chills, headache, myalgias, arthralgias, mild alopecia, psychiatric effects and associated disorders, autoimmune phenomena and associated disorders and thyroid dysfunction. Ribavirin, an inhibitor of inosine 5′-monophosphate dehydrogenase (IMPDH), enhances the efficacy of IFN-alpha in the treatment of HCV. Despite the introduction of ribavirin, more than 50% of the patients do not eliminate the virus with the current standard therapy of interferon-alpha (IFN) and ribavirin. By now, standard therapy of chronic hepatitis C has been changed to the combination of pegylated IFN-alpha plus ribavirin. However, a number of patients still have significant side effects, primarily related to ribavirin. Ribavirin causes significant hemolysis in 10-20% of patients treated at currently recommended doses, and the drug is both teratogenic and embryotoxic. Even with recent improvements, a substantial fraction of patients do not respond with a sustained reduction in viral load⁵ and there is a clear need for more effective antiviral therapy of HCV infection.

A number of approaches are being pursued to combat the virus. These include, for example, application of antisense oligonucleotides or ribozymes for inhibiting HCV replication. Furthermore, low-molecular weight compounds that directly inhibit HCV proteins and interfere with viral replication are considered as attractive strategies to control HCV infection. Among the viral targets, the NS3/4a protease/helicase and the NS5b RNA-dependent RNA polymerase are considered the most promising viral targets for new drugs.⁶⁻⁸

Besides targeting viral genes and their transcription and translation products, antiviral activity can also be achieved by targeting host cell proteins that are necessary for viral replication. For example, Watashi et al.⁹ show how antiviral activity can be achieved by inhibiting host cell cyclophilins. Alternatively, a potent TLR7 agonist has been shown to reduce HCV plasma levels in humans.¹⁰

However, none of the compounds described above have progressed beyond clinical trials.^{6,8}

In view of the worldwide epidemic level of HCV and other members of the Flaviviridae family of viruses, and further in view of the limited treatment options, there is a strong need for new effective drugs for treating infections cause by these viruses.

### SUMMARY OF THE INVENTION

In one embodiment, the present invention provides a compound that is Formula (I):

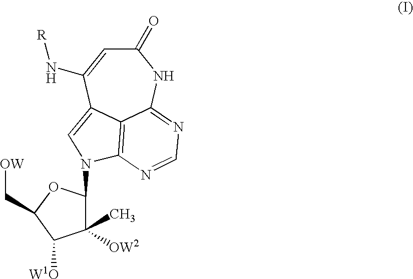

or a pharmaceutically acceptable salt or solvate thereof, wherein
R is selected from the group consisting of H and R¹(CO);
R¹ is selected from the group consisting of C₁₋₆ alkoxy, phenyl(C₁₋₆ alkoxy), substituted phenyl(C₁₋₆ alkoxy), (C₁₋₆ alkyl)(CO)O(C₁₋₆ alkoxy), substituted (C₁₋₆ alkyl)(CO)O(C₁₋₆ alkoxy), heterocyclyl(C₁₋₆ alkoxy), substituted heterocyclyl(C₁₋₆ alkoxy), amino(C₁₋₆ alkyl), substituted amino(C₁₋₆ alkyl), and acylamino(C₁₋₆ alkyl);
W and W¹ are independently selected from the group consisting of H, C₁₋₆ alkyl(CO), amino(C₁₋₆ alkyl)(CO), substituted amino(C₁₋₆ alkyl)(CO), acylamino(C₁₋₆ alkyl)(CO), heterocyclyl(C₁₋₆ alkyl)(CO), substituted heterocyclyl(C₁₋₆ alkyl)(CO), (C₁₋₆ alkyl)(CO)O(C₁₋₆ alkoxy), and substituted (C₁₋₆ alkyl)(CO)O(C₁₋₆ alkoxy);
W² is selected from the group consisting of H and C₁₋₆ alkyl(CO), heterocyclyl(C₁₋₆ alkyl)(CO); or OW¹ and OW² and together form a —O(CO)O— group; and
provided that when W, W¹, and W² are H, then R is not H or CH₃(CO).

In one embodiment, the provided is a compound that is Formula (Ia):

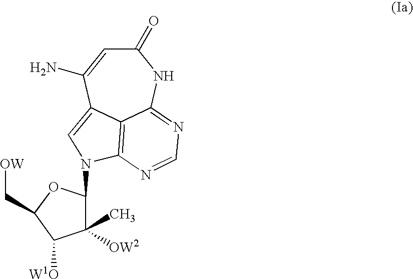

or a pharmaceutically acceptable salt or solvate thereof, wherein W, W¹, and W² are as defined for Formula (I).

In one embodiment, the provided is a compound that is Formula (Ib):

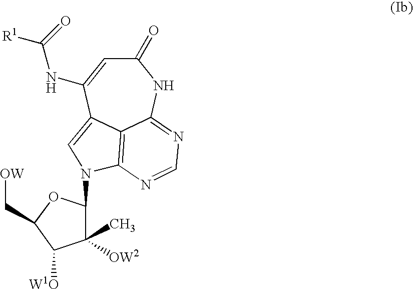

or a pharmaceutically acceptable salt or solvate thereof, wherein R¹ is selected from the group consisting of C₁₋₆ alkoxy, phenyl(C₁₋₆ alkoxy), substituted phenyl(C₁₋₆ alkoxy), (C₁₋₆ alkyl)(CO)O(C₁₋₆ alkoxy), substituted (C₁₋₆ alkyl)(CO)O(C₁₋₆ alkoxy), heterocyclyl(C₁₋₆ alkoxy), and substituted heterocyclyl(C₁₋₆ alkoxy); and W, W¹, and W² are as defined in Formula (I).

In one embodiment provided is a pharmaceutical composition comprising a pharmaceutically acceptable carrier and a therapeutically effective amount of a compound of Formula (I).

In other embodiments provided are methods for preparing the compounds and compositions of Formula (I) and for their therapeutic uses. In one embodiment provided is a method for treating a viral infection in a patient mediated at least in part by a virus in the Flaviviridae family of viruses, comprising administering to said patient a composition of Formula (I). In some aspects, the viral infection is mediated by hepatitis C virus.

These and other embodiments of the invention are further described in the text that follows.

### DETAILED DESCRIPTION OF THE INVENTION

Throughout this application, references are made to various embodiments relating to compounds, compositions, and methods. The various embodiments described are meant to provide a variety illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

### DEFINITIONS

It is to be understood that the terminology used herein is for the purpose of describing particular embodiments only and is not intended to limit the scope of the present invention. In this specification and in the claims that follow, reference will be made to a number of terms that shall be defined to have the following meanings:

“Alkyl” refers to monovalent saturated aliphatic hydrocarbyl groups having from 1 to 10 carbon atoms and, in some embodiments, from 1 to 6 carbon atoms. “C₁₋₆alkyl” refers to alkyl groups having from 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Substituted alkyl” refers to an alkyl group having from 1 to 5 and, in some embodiments, 1 to 3 or 1 to 2 substituents selected from the group consisting of alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, spirocycloalkyl, SO₃H, substituted sulfonyl, sulfonyloxy, thioacyl, thiocyanate, thiol, alkylthio, and substituted alkylthio, wherein said substituents are as defined herein.

“Alkenyl” refers to a linear or branched hydrocarbyl group having from 2 to 10 carbon atoms and in some embodiments from 2 to 6 carbon atoms or 2 to 4 carbon atoms and having at least 1 site of vinyl unsaturation (>C═C<). For example, (Cₓ-C_{y})alkenyl refers to alkenyl groups having from x to y carbon atoms and is meant to include for example, ethenyl, propenyl, 1,3-butadienyl, and the like.

“Substituted alkenyl” refers to alkenyl groups having from 1 to 3 substituents and, in some embodiments, 1 to 2 substituents selected from the group consisting of alkoxy, substituted alkoxy, acyl, acylamino, acyloxy, alkyl, substituted alkyl, alkynyl, substituted alkynyl, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to a vinyl (unsaturated) carbon atom.

“Alkynyl” refers to a linear monovalent hydrocarbon radical or a branched monovalent hydrocarbon radical containing at least one triple bond. The term “alkynyl” is also meant to include those hydrocarbyl groups having one triple bond and one double bond. For example, (C₂-C₆)alkynyl is meant to include ethynyl, propynyl, and the like.

“Substituted alkynyl” refers to alkynyl groups having from 1 to 3 substituents and, in some embodiments, from 1 to 2 substituents selected from the group consisting of alkoxy, substituted alkoxy, acyl, acylamino, acyloxy, alkyl, substituted alkyl, alkenyl, substituted alkenyl, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are as defined herein and with the proviso that any hydroxy or thiol substitution is not attached to an acetylenic carbon atom.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is as defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, aryl-C(O)—, substituted aryl-C(O)—, substituted hydrazino-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, aryl, substituted aryl, substituted hydrazino, heteroaryl, substituted heteroaryl, heterocyclic, and substituted heterocyclic are as defined herein. Acyl includes the “acetyl” group CH₃C(O)—.

“Acylamino” refers to the groups —NR²⁰C(O)alkyl, —NR²⁰C(O)substituted alkyl, —NR²⁰C(O)cycloalkyl, —NR²⁰C(O)substituted cycloalkyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic wherein R²⁰ is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Amino” refers to the group —NH₂.

“Substituted amino” refers to the group —NR²¹R²² where R²¹ and R²² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, and —SO₂-substituted heterocyclic and wherein R²¹ and R²² are optionally joined together with the nitrogen bound thereto to form a heterocyclic or substituted heterocyclic group, provided that R²¹ and R²² are both not hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein. When R²¹ is hydrogen and R²² is alkyl, the substituted amino group is sometimes referred to herein as alkylamino. When R²¹ and R²² are alkyl, the substituted amino group is sometimes referred to herein as dialkylamino. When referring to a monosubstituted amino, it is meant that either R²¹ or R²² is hydrogen but not both. When referring to a disubstituted amino, it is meant that neither R²¹ nor R²² are hydrogen.

“Hydroxyamino” refers to the group —NHOH.

“Alkoxyamino” refers to the group —NHO-alkyl wherein alkyl is defined herein.

“Aminocarbonyl” refers to the group —C(O)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, hydroxy, alkoxy, substituted alkoxy, amino, substituted amino, and acylamino, and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminothiocarbonyl” refers to the group —C(S)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminocarbonylamino” refers to the group —NR²⁰C(O)NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminothiocarbonylamino” refers to the group —NR²⁰C(S)NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminocarbonyloxy” refers to the group —O—C(O)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonyl” refers to the group —SO₂NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonyloxy” refers to the group —O—SO₂NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonylamino” refers to the group —NR²⁰—SO₂NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Amidino” refers to the group —C(═NR²⁵)NR²³R²⁴ where R²⁵, R²³, and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aryl” or “Ar” refers to an aromatic group of from 6 to 14 carbon atoms and no ring heteroatoms and having a single ring (e.g., phenyl) or multiple condensed (fused) rings (e.g., naphthyl or anthryl). For multiple ring systems, including fused, bridged, and spiro ring systems having aromatic and non-aromatic rings that have no ring heteroatoms, the term “Aryl” or “Ar” applies when the point of attachment is at an aromatic carbon atom (e.g., 5,6,7,8 tetrahydronaphthalene-2-Yl is an aryl group as its point of attachment is at the 2-position of the aromatic phenyl ring).

“Substituted aryl” refers to aryl groups which are substituted with 1 to 8 and, in some embodiments, 1 to 5, 1 to 3, or 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiocyanate, thiol, alkylthio, and substituted alkylthio, wherein said substituents are defined herein.

“Aryloxy” refers to the group —O-aryl, where aryl is as defined herein, that includes, by way of example, phenoxy and naphthyloxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Azido” refers to the group —N₃.

“Hydrazino” refers to the group —NHNH₂.

“Substituted hydrazino” refers to the group —NR²⁶NR²⁷R²⁸ where R²⁶, R²⁷, and R²⁸ are independently selected from the group consisting of hydrogen, alkyl, substituted alkyl, alkenyl, substituted alkenyl, alkynyl, substituted alkynyl, aryl, substituted aryl, carboxyl ester, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, and —SO₂-substituted heterocyclic and wherein R²⁷ and R²⁸ are optionally joined, together with the nitrogen bound thereto to form a heterocyclic or substituted heterocyclic group, provided that R²⁷ and R²⁸ are both not hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Cyano” or “carbonitrile” refers to the group —CN.

“Carbonyl” refers to the divalent group —C(O)— which is equivalent to —C(═O)—.

“Carboxyl” or “carboxy” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)amino” refers to the group —NR²⁰—C(O)O-alkyl, —NR¹⁰—C(O)O-substituted alkyl, —NR²⁰—C(O)O-alkenyl, —NR²⁰—C(O)O-substituted alkenyl, —NR²⁰—C(O)O-alkynyl, —NR²⁰—C(O)O-substituted alkynyl, —NR²⁰—C(O)O-aryl, —NR²⁰—C(O)O-substituted aryl, —NR²⁰—C(O)O-cycloalkyl, —NR²⁰—C(O)O-substituted cycloalkyl, —NR²⁰—C(O)O-heteroaryl, —NR²⁰—C(O)O-substituted heteroaryl, —NR²⁰—C(O)O-heterocyclic, and —NR²⁰—C(O)O-substituted heterocyclic wherein R²⁰ is alkyl or hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Cycloalkyl” refers to a saturated or partially saturated cyclic group of from 3 to 14 carbon atoms and no ring heteroatoms and having a single ring or multiple rings including fused, bridged, and spiro ring systems. For multiple ring systems having aromatic and non-aromatic rings that have no ring heteroatoms, the term “cycloalkyl” applies when the point of attachment is at a non-aromatic carbon atom (e.g. 5,6,7,8,-tetrahydronaphthalene-5-yl). The term “Cycloalkyl” includes cycloalkenyl groups. Examples of cycloalkyl groups include, for instance, adamantyl, cyclopropyl, cyclobutyl, cyclopentyl, cyclooctyl, and cyclohexenyl. “Cᵤ₋ᵥcycloalkyl” refers to cycloalkyl groups having u to v carbon atoms.

“Cycloalkenyl” refers to a partially saturated cycloalkyl ring having at least one site of >C═C< ring unsaturation.

“Cycloalkylene” refer to divalent cycloalkyl groups as defined herein. Examples of cycloalkyl groups include those having three to six carbon ring atoms such as cyclopropylene, cyclobutylene, cyclopentylene, and cyclohexylene.

“Substituted cycloalkyl” refers to a cycloalkyl group, as defined herein, having from 1 to 8, or 1 to 5, or in some embodiments 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiocyanate, thiol, alkylthio, and substituted alkylthio, wherein said substituents are as defined herein. The term “substituted cycloalkyl” includes substituted cycloalkenyl groups.

“Cycloalkyloxy” refers to —O-cycloalkyl wherein cycloalkyl is as defined herein.

“Substituted cycloalkyloxy refers to —O-(substituted cycloalkyl) wherein substituted cycloalkyl is as defined herein.

“Cycloalkylthio” refers to —S-cycloalkyl wherein cycloalkyl is as defined herein.

“Substituted cycloalkylthio” refers to —S-(substituted cycloalkyl).

“Guanidino” refers to the group —NHC(═NH)NH₂.

“Substituted guanidino” refers to —NR²⁹C(═NR²⁹)N(R²⁹)₂ where each R²⁹ is independently selected from the group consisting of hydrogen, alkyl, substituted alkyl, aryl, substituted aryl, heteroaryl, substituted heteroaryl, heterocyclyl, and substituted heterocyclyl and two R²⁹ groups attached to a common guanidino nitrogen atom are optionally joined together with the nitrogen bound thereto to form a heterocyclic or substituted heterocyclic group, provided that at least one R²⁹ is not hydrogen, and wherein said substituents are as defined herein.

“Halo” or “halogen” refers to fluoro, chloro, bromo, and iodo.

“Haloalkyl” refers to substitution of alkyl groups with 1 to 5 or in some embodiments 1 to 3 halo groups.

“Haloalkoxy” refers to substitution of alkoxy groups with 1 to 5 or in some embodiments 1 to 3 halo groups.

“Hydroxy” or “hydroxyl” refers to the group —OH.

“Heteroaryl” refers to an aromatic group of from 1 to 14 carbon atoms and 1 to 6 heteroatoms selected from the group consisting of oxygen, nitrogen, and sulfur and includes single ring (e.g. imidazolyl) and multiple ring systems (e.g. benzimidazol-2-yl and benzimidazol-6-yl). For multiple ring systems, including fused, bridged, and spiro ring systems having aromatic and non-aromatic rings, the term “heteroaryl” applies if there is at least one ring heteroatom and the point of attachment is at an atom of an aromatic ring (e.g. 1,2,3,4-tetrahydroquinolin-6-yl and 5,6,7,8-tetrahydroquinolin-3-yl). In one embodiment, the nitrogen and/or the sulfur ring atom(s) of the heteroaryl group are optionally oxidized to provide for the N-oxide (N→O), sulfinyl, or sulfonyl moieties. More specifically the term heteroaryl includes, but is not limited to, pyridyl, furanyl, thienyl, thiazolyl, isothiazolyl, triazolyl, imidazolyl, isoxazolyl, pyrrolyl, pyrazolyl, pyridazinyl, pyrimidinyl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, quinazolinonyl, benzimidazolyl, benzisoxazolyl, or benzothienyl.

“Substituted heteroaryl” refers to heteroaryl groups that are substituted with from 1 to 8 or in some embodiments 1 to 5, or 1 to 3, or 1 to 2 substituents selected from the group consisting of the substituents defined for substituted aryl.

“Heteroaryloxy” refers to —O-heteroaryl wherein heteroaryl is as defined herein.

“Substituted heteroaryloxy refers to the group —O-(substituted heteroaryl) wherein substituted heteroaryl is as defined herein.

“Heteroarylthio” refers to the group —S-heteroaryl wherein heteroaryl is as defined herein.

“Substituted heteroarylthio” refers to the group —S-(substituted heteroaryl) wherein substituted heteroaryl is as defined herein.

“Heterocyclic” or “heterocycle” or “heterocycloalkyl” or “heterocyclyl” refers to a saturated or partially saturated cyclic group having from 1 to 14 carbon atoms and from 1 to 6 heteroatoms selected from the group consisting of nitrogen, sulfur, or oxygen and includes single ring and multiple ring systems including fused, bridged, and spiro ring systems. For multiple ring systems having aromatic and/or non-aromatic rings, the terms “heterocyclic”, “heterocycle”, “heterocycloalkyl”, or “heterocyclyl” apply when there is at least one ring heteroatom and the point of attachment is at an atom of a non-aromatic ring (e.g. 1,2,3,4-tetrahydroquinoline-3-yl, 5,6,7,8-tetrahydroquinoline-6-yl, and decahydroquinolin-6-yl). In one embodiment, the nitrogen and/or sulfur atom(s) of the heterocyclic group are optionally oxidized to provide for the N-oxide, sulfinyl, sulfonyl moieties. More specifically the heterocyclyl includes, but is not limited to, tetrahydropyranyl, piperidinyl, N-methylpiperidin-3-yl, piperazinyl, N-methylpyrrolidin-3-yl, 3-pyrrolidinyl, 2-pyrrolidon-1-yl, morpholinyl, and pyrrolidinyl. A prefix indicating the number of carbon atoms (e.g., C₃-C₁₀) refers to the total number of carbon atoms in the portion of the heterocyclyl group exclusive of the number of heteroatoms.

“Substituted heterocyclic” or “Substituted heterocycle” or “substituted heterocycloalkyl” or “substituted heterocyclyl” refers to heterocyclic groups, as defined herein, that are substituted with from 1 to 5 or in some embodiments 1 to 3 of the substituents as defined for substituted cycloalkyl.

“Heterocyclyloxy” refers to the group —O-heterocycyl wherein heterocyclyl is as defined herein.

“Substituted heterocyclyloxy” refers to the group —O-(substituted heterocycyl) wherein substituted heterocyclyl is as defined herein.

“Heterocyclylthio” refers to the group —S-heterocycyl wherein heterocyclyl is as defined herein.

“Substituted heterocyclylthio” refers to the group —S-(substituted heterocycyl) wherein substituted heterocyclyl is as defined herein.

Examples of heterocycle and heteroaryl groups include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O).

“Oxide” refers to products resulting from the oxidation of one or more heteroatoms. Examples include N-oxides, sulfoxides, and sulfones.

“Spirocycloalkyl” refers to a 3 to 10 member cyclic substituent formed by replacement of two hydrogen atoms at a common carbon atom with an alkylene group having 2 to 9 carbon atoms, as exemplified by the following structure wherein the methylene group shown here attached to bonds marked with wavy lines is substituted with a spirocycloalkyl group:

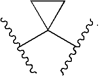

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-alkynyl, —SO₂-substituted alkynyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein. Substituted sulfonyl includes groups such as methyl-SO₂—, phenyl-SO₂—, and 4-methylphenyl-SO₂—.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cylcoalkyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thiol” refers to the group —SH.

“Alkylthio” refers to the group —S-alkyl wherein alkyl is as defined herein.

“Substituted alkylthio” refers to the group —S-(substituted alkyl) wherein substituted alkyl is as defined herein.

“Thiocarbonyl” refers to the divalent group —C(S)— which is equivalent to —C(═S)—.

“Thione” refers to the atom (═S).

“Thiocyanate” refers to the group —SCN.

“Compound” and “compounds” as used herein refers to a compound encompassed by the generic formulae disclosed herein, any subgenus of those generic formulae, and any forms of the compounds within the generic and subgeneric formulae, including the racemates, stereoisomers, and tautomers of the compound or compounds.

“Racemates” refers to a mixture of enantiomers.

“Solvate” or “solvates” of a compound refer to those compounds, where compounds is as defined above, that are bound to a stoichiometric or non-stoichiometric amount of a solvent. Solvates of a compound includes solvates of all forms of the compound. Preferred solvents are volatile, non-toxic, and/or acceptable for administration to humans in trace amounts. Suitable solvates include water.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

“Patient” refers to mammals and includes humans and non-human mammals.

“Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

Unless indicated otherwise, the nomenclature of substituents that are not explicitly defined herein are arrived at by naming the terminal portion of the functionality followed by the adjacent functionality toward the point of attachment. For example, the substituent “arylalkyloxycabonyl” refers to the group (aryl)-(alkyl)-O—C(O)—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

Accordingly in one embodiment, provided is a compound that is Formula (I):

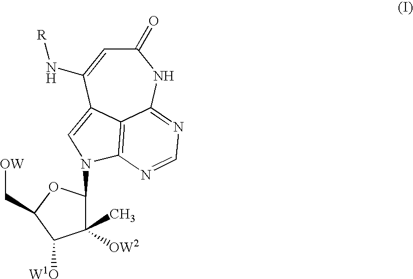

or a pharmaceutically acceptable salt or solvate thereof, wherein
R is selected from the group consisting of H and R¹(CO);
R¹ is selected from the group consisting of C₁₋₆ alkoxy, phenyl(C₁₋₆ alkoxy), substituted phenyl(C₁₋₆ alkoxy), (C₁₋₆ alkyl)(CO)O(C₁₋₆ alkoxy), substituted (C₁₋₆ alkyl)(CO)O(C₁₋₆ alkoxy), heterocyclyl(C₁₋₆ alkoxy), substituted heterocyclyl(C₁₋₆ alkoxy), amino(C₁₋₆ alkyl), substituted amino(C₁₋₆ alkyl), and acylamino(C₁₋₆ alkyl);
W and W¹ are independently selected from the group consisting of H, C₁₋₆ alkyl(CO), amino(C₁₋₆ alkyl)(CO), substituted amino(C₁₋₆ alkyl)(CO), acylamino(C₁₋₆ alkyl)(CO), heterocyclyl(C₁₋₆ alkyl)(CO), substituted heterocyclyl(C₁₋₆ alkyl)(CO), (C₁₋₆ alkyl)(CO)O(C₁₋₆ alkoxy), and substituted (C₁₋₆ alkyl)(CO)O(C₁₋₆ alkoxy);
W² is selected from the group consisting of H and C₁₋₆ alkyl(CO), heterocyclyl(C₁₋₆ alkyl)(CO); or OW¹ and OW² and together form a —O(CO)O— group; and
provided that when W, W¹, and W² are H, then R is not H or CH₃(CO).

In one embodiment, the provided is a compound that is Formula (Ia):

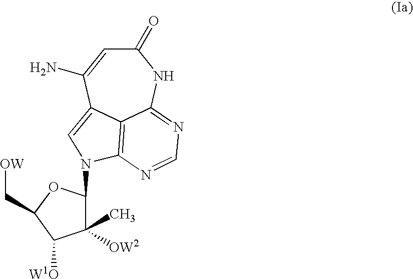

or a pharmaceutically acceptable salt or solvate thereof, wherein W, W¹, and W² are as defined for Formula (I).

In one embodiment, the provided is a compound that is Formula (Ib):

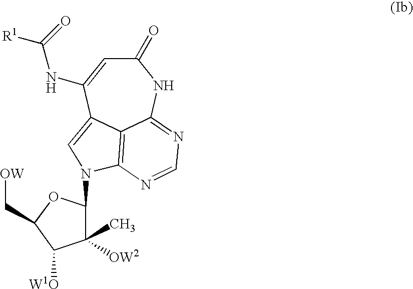

or a pharmaceutically acceptable salt or solvate thereof, wherein R¹ is selected from the group consisting of C₁₋₆ alkoxy, phenyl(C₁₋₆ alkoxy), substituted phenyl(C₁₋₆ alkoxy), (C₁₋₆ alkyl)(CO)O(C₁₋₆ alkoxy), substituted (C₁₋₆ alkyl)(CO)O(C₁₋₆ alkoxy), heterocyclyl(C₁₋₆ alkoxy), and substituted heterocyclyl(C₁₋₆ alkoxy); and W, W¹, and W² are as defined in Formula (I).

In some embodiments, R¹ is (C₁₋₆ alkyl)(CO)O(C₁₋₆ alkoxy).

In some embodiments, R¹ is (CH₃)₂CH(CO)OCH₂O—.

In some embodiments, R¹ is amino(C₁₋₆ alkyl).

In some embodiments, R¹ is substituted heterocyclyl(C₁₋₆ alkoxy).

In some embodiments, R¹ is amino(C₁₋₆ alkyl)(CO)O(C₁₋₆ alkoxy).

In some embodiments, R¹ is substituted amino(C₁₋₆ alkyl)(CO)O(C₁₋₆ alkoxy).

In some embodiments, R¹ is acylamino(C₁₋₆ alkyl)(CO)O(C₁₋₆ alkoxy).

In one embodiment, provided is a compound that is a pharmaceutically acceptable salt of Formula (I).

In one embodiment, provided is a compound that is a solvate of Formula (I). In some aspects, the solvate is a solvate of a pharmaceutically acceptable salt of Formula (I).

Various features relating to the embodiments above are given below. These features when referring to different substituents or variables can be combined with each other or with any other embodiments described in this application. In some aspects, provided are compounds of Formula (I), (Ia), or (Ib) having one or more of the following features below.

In some embodiments, at least one of W, W¹, or W² is C₁₋₆ alkyl(CO).

In some embodiments, W and W¹ are independently C₁₋₆ alkyl(CO).

In some embodiments, W, W¹, and W² are independently C₁₋₆ alkyl(CO).

In some embodiments, W, W¹, and W² are independently selected from the group consisting of CH₃(CO), CH₃CH₂(CO), and (CH₃)₂CH(CO).

In some embodiments, W, W¹, and W are CH₃(CO).

In some embodiments, W, W¹, and W² are CH₃CH₂(CO).

In some embodiments, W, W¹, and W² are (CH₃)₂CH(CO).

In some embodiments, W is H.

In some embodiments, W² is H.

In some embodiments, W¹ and W² are H.

In some embodiments, OW¹ and OW² together form a —O(CO)O— group.

In other embodiments, provided is a compound selected from Table 1 or a pharmaceutically acceptable salt or solvate thereof.

TABLE 1

| Cmpd | Structure | Name |
|---|---|---|
| 101 | 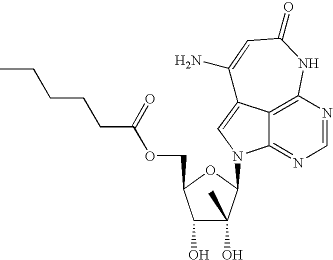 | Hexanoic acid 5-(9-amino-7-oxo-6,7-dihydro-2,3,5,6-tetraaza-benzo[cd]azulen-2-yl)-3,4-dihydroxy-4-methyl-tetrahydro-furan-2-ylmethylester |
| 102 | 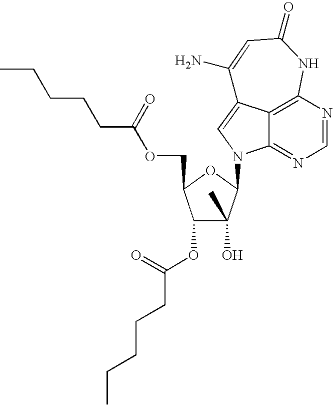 | Hexanoic acid 5-(9-amino-7-oxo-6,7-dihydro-2,3,5,6-tetraaza-benzo[cd]azulen-2-yl)-3-hexanoyloxy-4-hydroxy-4-methyl-tetrahydro-furan-2-ylmethyl ester |
| 103 | 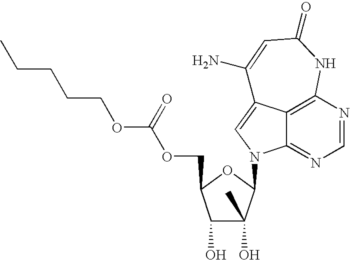 | Carbonic acid 5-(9-amino-7-oxo-6,7-dihydro-2,3,5,6-tetraaza-benzo[cd]azulen-2-yl)-3,4-dihydroxy-4-methyl-tetrahydro-furan-2-ylmethylester pentyl ester |
| 104 | 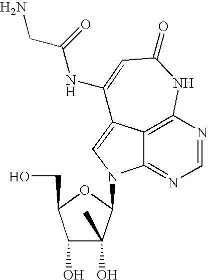 | 2-Amino-N-[2-(3,4-dihydroxy-5-hydroxymethyl-3-methyl-tetrahydro-furan-2-yl)-7-oxo-6,7-dihydro-2H-2,3,5,6-tetraaza-benzo[cd]azulen-9-yl]-acetamide |
| 105 | 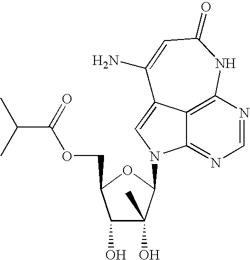 | Isobutyric acid 5-(9-amino-7-oxo-6,7-dihydro-2,3,5,6-tetraaza-benzo[cd]azulen-2-yl)-3,4-dihydroxy-4-methyl-tetrahydro-furan-2-ylmethylester |
| 106 | 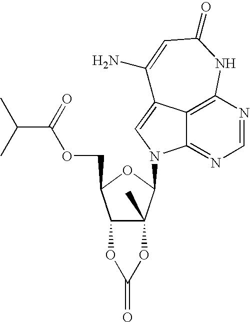 | Isobutyric acid 6-(9-amino-7-oxo-6,7-dihydro-2,3,5,6-tetraaza-benzo[cd]azulen-2-yl)-6a-methyl-2-oxo-tetrahydro-furo[3,4-d][1,3]dioxol-4-ylmethyl ester |
| 107 | 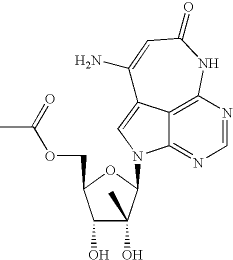 | Acetic acid 5-(9-amino-7-oxo-6,7-dihydro-2,3,5,6-tetraaza-benzo[cd]azulen-2-yl)-3,4-dihydroxy-4-methyl-tetrahydro-furan-2-ylmethylester |
| 108 | 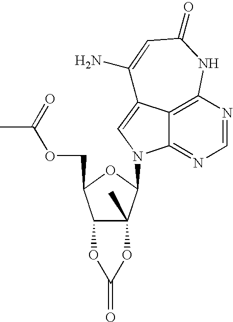 | Acetic acid 6-(9-amino-7-oxo-6,7-dihydro-2,3,5,6-tetraaza-benzo[cd]azulen-2-yl)-6a-methyl-2-oxo-tetrahydro-furo[3,4-d][1,3]dioxol-4-ylmethyl ester |
| 109 | 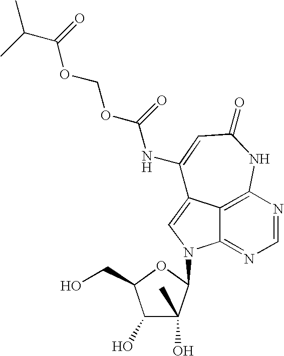 | Isobutyric acid 2-(3,4-dihydroxy-5-hydroxymethyl-3-methyl-tetrahydro-furan-2-yl)-7-oxo-6,7-dihydro-2H-2,3,5,6-tetraaza-benzo[cd]azulen-9-ylcarbamoyloxymethyl ester |
| 110 | 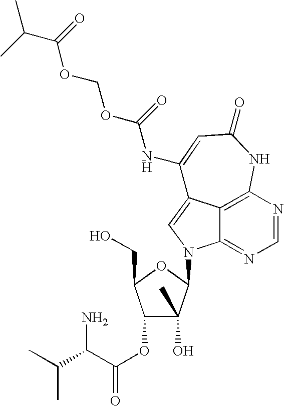 | 2-Amino-3-methyl-butyric acid 4-hydroxy-2-hydroxymethyl-5-(9-isobutyryloxymethoxycarbonyl-amino-7-oxo-6,7-dihydro-2,3,5,6-tetraaza-benzo[cd]azulen-2-yl)-4-methyl-tetrahydro-furan-3-yl ester |
| 111 | 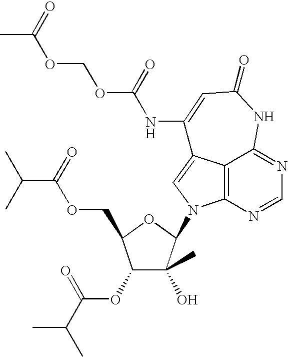 | Isobutyric acid 5-(9-acetoxymethoxycarbonylamino-7-oxo-6,7-dihydro-2,3,5,6-tetraaza-benzo[cd]azulen-2-yl)-4-hydroxy-2-isobutyryloxymethyl-4-methyl-tetrahydro-furan-3-yl ester |
| 112 | 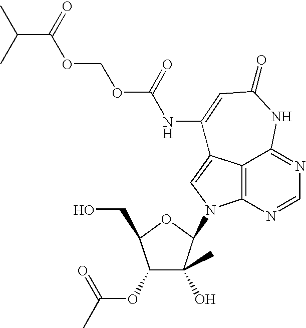 | Isobutyric acid 2-(4-acetoxy-3-hydroxy-5-hydroxymethyl-3-methyl-tetrahydro-furan-2-yl)-7-oxo-6,7-dihydro-2H-2,3,5,6-tetraaza-benzo[cd]azulen-9-ylcarbamoyloxymethyl ester |
| 113 | 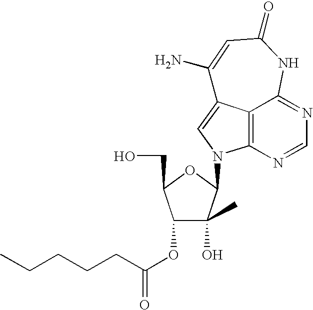 | Hexanoic acid 5-(9-amino-7-oxo-6,7-dihydro-2,3,5,6-tetraaza-benzo[cd]azulen-2-yl)-4-hydroxy-2-hydroxymethyl-4-methyl-tetrahydro-furan-3-yl ester |
| 114 | 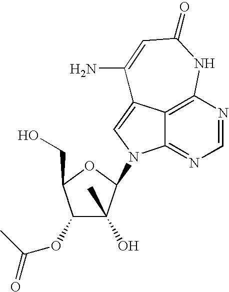 | Acetic acid 5-(9-amino-7-oxo-6,7-dihydro-2,3,5,6-tetraaza-benzo[cd]azulen-2-yl)-4-hydroxy-2-hydroxymethyl-4-methyl-tetrahydro-furan-3-yl ester |
| 115 | 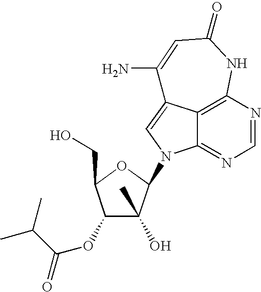 | Isobutyric acid 5-(9-amino-7-oxo-6,7-dihydro-2,3,5,6-tetraaza-benzo[cd]azulen-2-yl)-4-hydroxy-2-hydroxymethyl-4-methyl-tetrahydro-furan-3-yl ester |
| 116 | 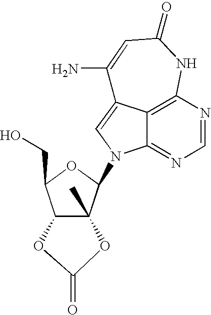 | 9-Amino-2-(6-hydroxymethyl-3a-methyl-2-oxo-tetrahydro-furo[3,4-d][1,3]dioxol-4-yl)-2,6-dihydro-2,3,5,6-tetraaza-benzo[cd]azulen-7-one |
| 117 | 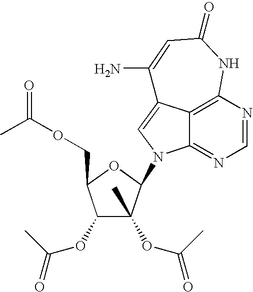 | Acetic acid 4-acetoxy-2-acetoxymethyl-5-(9-amino-7-oxo-6,7-dihydro-2,3,5,6-tetraaza-benzo[cd]azulen-2-yl)-4-methyl-tetrahydro-furan-3-yl ester |
| 118 | 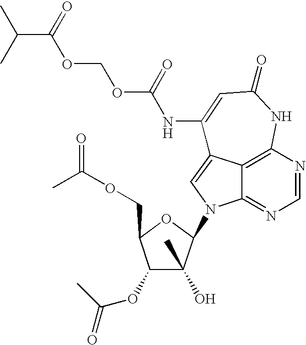 | Isobutyric acid 2-(4-acetoxy-5-acetoxymethyl-3-hydroxy-3-methyl-tetrahydro-furan-2-yl)-7-oxo-6,7-dihydro-2H-2,3,5,6-tetraaza-benzo[cd]azulen-9-ylcarbamoyloxymethyl ester |
| 119 | 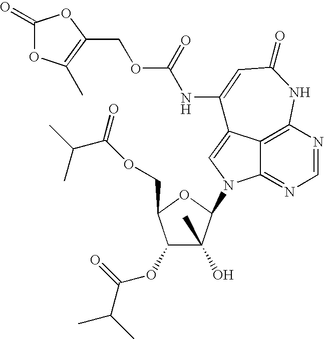 | Isobutyric acid 4-hydroxy-3-isobutyryloxy-4-methyl-5-[9-(5-methyl-2-oxo-[1,3]dioxol-4-ylmethoxycarbonylamino)-7-oxo-6,7-dihydro-2,3,5,6-tetraaza-benzo[cd]azulen-2-yl]-tetrahydro-furan-2-ylmethyl ester |
| 120 | 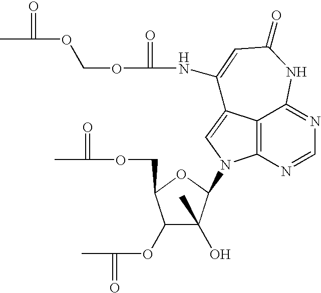 | Acetic acid 3-acetoxy-5-(9-acetoxymethoxycarbonylamino-7-oxo-6,7-dihydro-2,3,5,6-tetraaza-benzo[cd]azulen-2-yl)-4-hydroxy-4-methyl-tetrahydro-furan-2-ylmethylester |
| 121 | 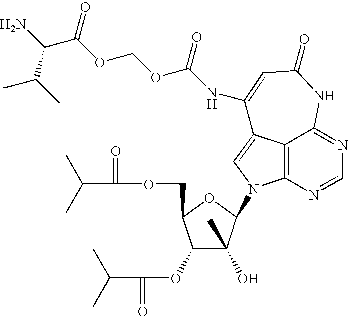 | 2-Amino-3-methyl-butyric acid 2-(3-hydroxy-4-isobutyryloxy-5-isobutyryloxymethyl-3-methyl-tetrahydro-furan-2-yl)-7-oxo-6,7-dihydro-2H-2,3,5,6-tetraaza-benzo[cd]azulen-9-ylcarbamoyloxymethyl ester |
| 122 | 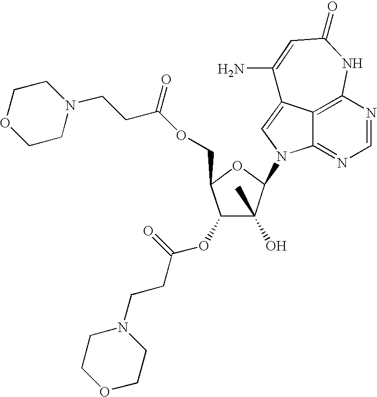 | 3-Morpholin-4-yl-propionic acid 5-(9-amino-7-oxo-6,7-dihydro-2,3,5,6-tetraaza-benzo[cd]azulen-2-yl)-4-hydroxy-4-methyl-3-(3-morpholin-4-yl-propionyloxy)-tetrahydro-furan-2-ylmethyl ester |
| 123 | 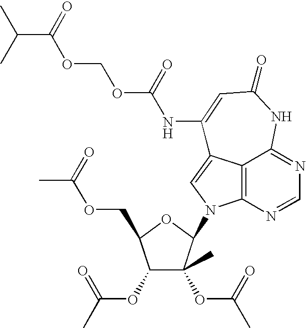 | Isobutyric acid 2-(3,4-diacetoxy-5-acetoxymethyl-3-methyl-tetrahydro-furan-2-yl)-7-oxo-6,7-dihydro-2H-2,3,5,6-tetraaza-benzo[cd]azulen-9-ylcarbamoyloxymethyl ester |
| 124 | 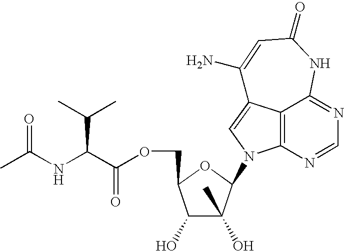 | 2-Acetylamino-3-methyl-butyric acid5-(9-amino-7-oxo-6,7-dihydro-2,3,5,6-tetraaza-benzo[cd]azulen-2-yl)-3,4-dihydroxy-4-methyl-tetrahydro-furan-2-ylmethyl ester |
| 125 | 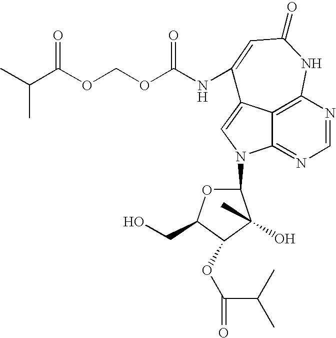 | Isobutyric acid 4-hydroxy-2-hydroxymethyl-5-(9-isobutyryloxymethoxycarbonyl-amino-7-oxo-6,7-dihydro-2,3,5,6-tetraaza-benzo[cd]azulen-2-yl)-4-methyl-tetrahydro-furan-3-yl ester |
| 126 | 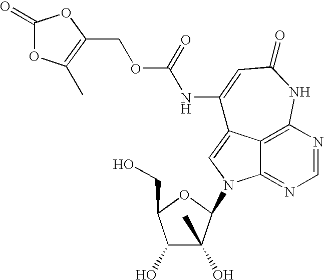 | [2-(3,4-Dihydroxy-5-hydroxymethyl-3-methyl-tetrahydro-furan-2-yl)-7-oxo-6,7-dihydro-2H-2,3,5,6-tetraaza-benzo[cd]azulen-9-yl]-carbamic acid5-methyl-2-oxo-[1,3]dioxol-4-ylmethyl ester |
| 127 | 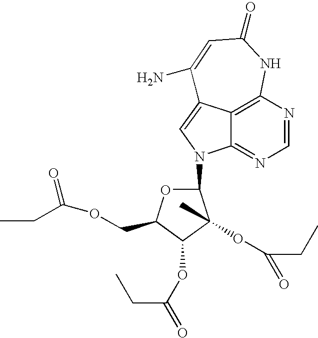 | Propionic acid 5-(9-amino-7-oxo-6,7-dihydro-2,3,5,6-tetraaza-benzo[cd]azulen-2-yl)-4-methyl-3,4-bis-propionyloxy-tetrahydro-furan-2-ylmethyl ester |
| 128 | 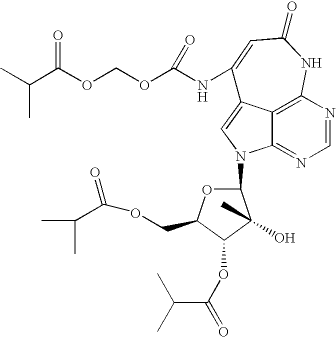 | Isobutyric acid 4-hydroxy-3-isobutyryloxy-5-(9-iso-butyryloxymethoxycarbonylamino-7-oxo-6,7-dihydro-2,3,5,6-tetraaza-benzo[cd]azulen-2-yl)-4-methyl-tetrahydro-furan-2-ylmethyl ester |
| 129 | 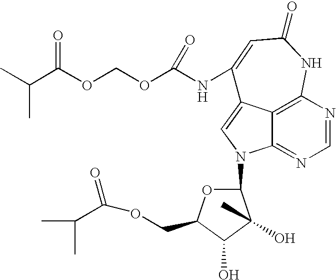 | Isobutyric acid 3,4-dihydroxy-5-(9-isobutyryloxymethoxycarbonyl-amino-7-oxo-6,7-dihydro-2,3,5,6-tetraaza-benzo[cd]azulen-2-yl)-4-methyl-tetrahydro-furan-2-ylmethylester |
| 130 | 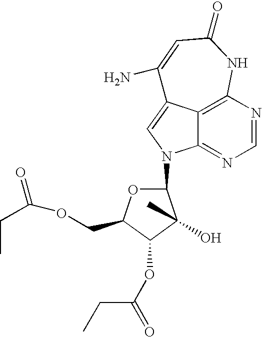 | Propionic acid 5-(9-amino-7-oxo-6,7-dihydro-2,3,5,6-tetraaza-benzo[cd]azulen-2-yl)-4-hydroxy-4-methyl-3-propionyloxy-tetrahydro-furan-2-ylmethyl ester |
| 131 | 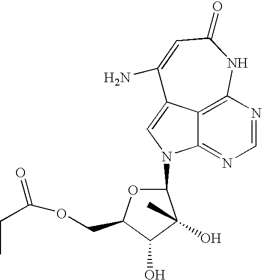 | Propionic acid 5-(9-amino-7-oxo-6,7-dihydro-2,3,5,6-tetraaza-benzo[cd]azulen-2-yl)-3,4-dihydroxy-4-methyl-tetrahydro-furan-2-ylmethylester |
| 132 | 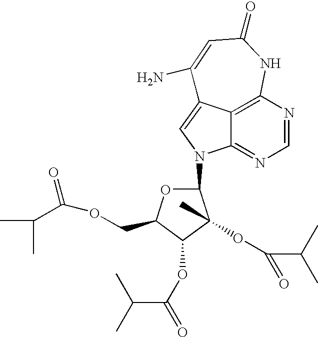 | Isobutyric acid 5-(9-amino-7-oxo-6,7-dihydro-2,3,5,6-tetraaza-benzo[cd]azulen-2-yl)-3,4-bis-isobutyryloxy-4-methyl-tetrahydro-furan-2-ylmethyl ester |
| 133 | 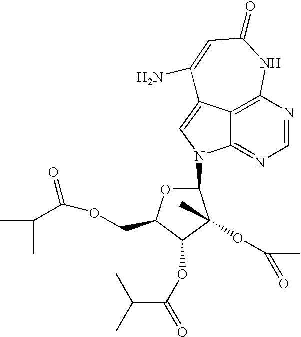 | Isobutyric acid 4-acetoxy-5-(9-amino-7-oxo-6,7-dihydro-2,3,5,6-tetraaza-benzo[cd]azulen-2-yl)-3-isobutyryloxy-4-methyl-tetrahydro-furan-2-ylmethyl ester |
| 134 | 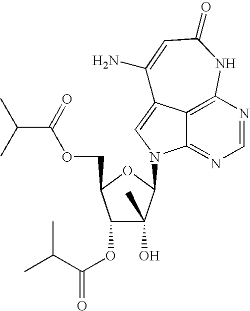 | Isobutyric acid 5-(9-amino-7-oxo-6,7-dihydro-2,3,5,6-tetraaza-benzo[cd]azulen-2-yl)-4-hydroxy-3-isobutyryloxy-4-methyl-tetrahydro-furan-2-ylmethyl ester |
| 135 | 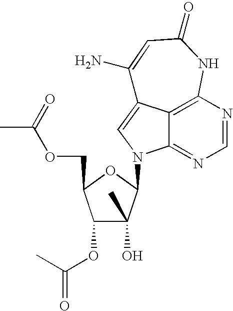 | Acetic acid 3-acetoxy-5-(9-amino-7-oxo-6,7-dihydro-2,3,5,6-tetraaza-benzo[cd]azulen-2-yl)-4-hydroxy-4-methyl-tetrahydro-furan-2-ylmethylester |
| 136 | 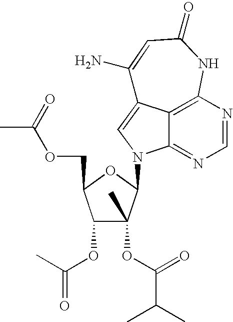 | Isobutyric acid 4-acetoxy-5-acetoxymethyl-2-(9-amino-7-oxo-6,7-dihydro-2,3,5,6-tetraaza-benzo[cd]azulen-2-yl)-3-methyl-tetrahydro-furan-3-yl ester |
| 138 | 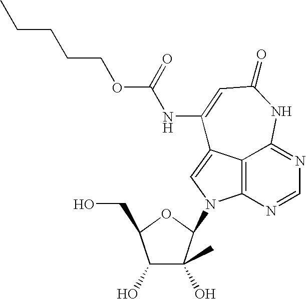 | [2-(3,4-Dihydroxy-5-hydroxymethyl-3-methyl-tetrahydro-furan-2-yl)-7-oxo-6,7-dihydro-2H-2,3,5,6-tetraaza-benzo[cd]azulen-9-yl]-carbamic acidpentyl ester |
| 139 | 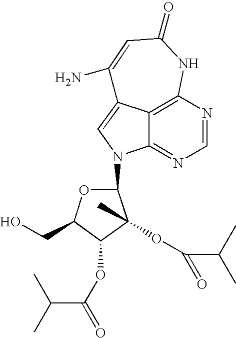 | Isobutyric acid 5-(9-amino-7-oxo-6,7-dihydro-2,3,5,6-tetraaza-benzo[cd]azulen-2-yl)-2-hydroxymethyl-4-isobutyryloxy-4-methyl-tetrahydro-furan-3-yl ester |
| 140 | 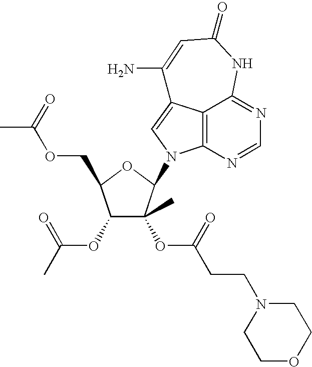 | 3-Morpholin-4-yl-propionic acid 4-acetoxy-5-acetoxymethyl-2-(9-amino-7-oxo-6,7-dihydro-2,3,5,6-tetraaza-benzo[cd]azulen-2-yl)-3-methyl-tetrahydro-furan-3-yl ester |
| 141 | 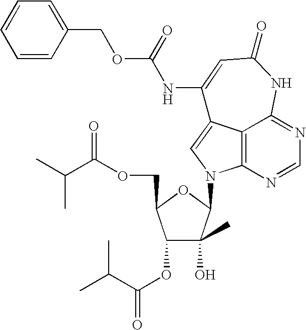 | Isobutyric acid 5-(9-benzyloxycarbonylamino-7-oxo-6,7-dihydro-2,3,5,6-tetraaza-benzo[cd]azulen-2-yl)-4-hydroxy-3-isobutyryloxy-4-methyl-tetrahydro-furan-2-ylmethyl ester |
| 142 | 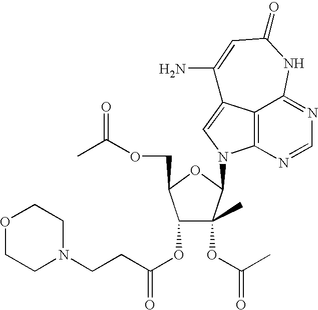 | 3-Morpholin-4-yl-propionic acid 4-acetoxy-2-acetoxymethyl-5-(9-amino-7-oxo-6,7-dihydro-2,3,5,6-tetraaza-benzo[cd]azulen-2-yl)-4-methyl-tetrahydro-furan-3-yl ester |
| 143 | 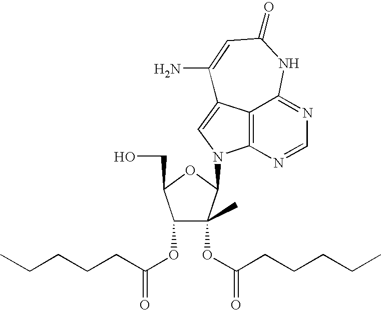 | Hexanoic acid 5-(9-amino-7-oxo-6,7-dihydro-2,3,5,6-tetraaza-benzo[cd]azulen-2-yl)-4-hexanoyloxy-2-hydroxymethyl-4-methyl-tetrahydro-furan-3-yl ester |
| 144 | 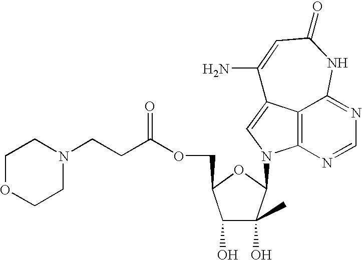 | 3-Morpholin-4-yl-propionic acid 5-(9-amino-7-oxo-6,7-dihydro-2,3,5,6-tetraaza-benzo[cd]azulen-2-yl)-3,4-dihydroxy-4-methyl-tetrahydro-furan-2-ylmethyl ester |
| 145 | 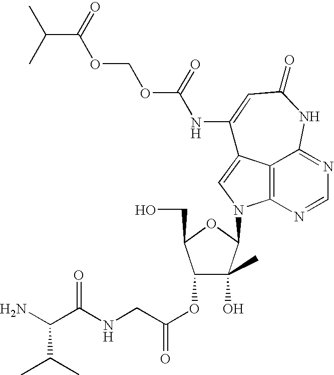 | Isobutyric acid 2-{4-[2-(2-amino-3-methyl-butyrylamino)-acetoxy]-3-hydroxy-5-hydroxymethyl-3-methyl-tetrahydro-furan-2-yl}-7-oxo-6,7-dihydro-2H-2,3,5,6-tetraaza-benzo[cd]azulen-9-ylcarbamoyloxymethyl ester |
| 146 | 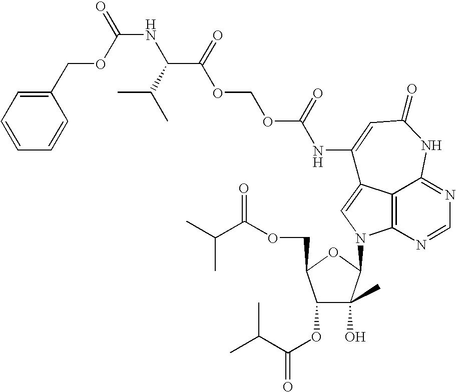 | 2-Benzyloxycarbonylamino-3-methyl-butyric acid 2-(3-hydroxy-4-isobutyryloxy-5-isobutyryloxymethyl-3-methyl-tetrahydro-furan-2-yl)-7-oxo-6,7-dihydro-2H-2,3,5,6-tetraaza-benzo[cd]azulen-9-ylcarbamoyloxymethyl ester |

In other embodiments, provided are pharmaceutical compositions comprising a pharmaceutically acceptable diluent and a therapeutically effective amount of one of the compounds described herein or mixtures of one or more of such compounds.

In other embodiments, provided are methods for treating in patients a viral infection mediated at least in part by a virus in the Flaviviridae family of viruses, such as HCV, which methods comprise administering to a patient that has been diagnosed with said viral infection or is at risk of developing said viral infection a pharmaceutical composition comprising a pharmaceutically acceptable diluent and a therapeutically effective amount of one of the compounds described herein or mixtures of one or more of such compounds. In another aspect, present provided are use of the compounds of Formula (I) for the preparation of a medicament for treating or preventing said infections. In other aspects the patient is a human.

In yet another embodiment provided are methods of treating or preventing viral infections in patients in combination with the administration of a therapeutically effective amount of one or more agents active against HCV. Active agents against HCV include ribavirin, levovirin, viramidine, thymosin alpha-1, an inhibitor of NS3 serine protease, and inhibitor of inosine monophosphate dehydrogenase, interferon-alpha, pegylated interferon-alpha, alone or in combination with ribavirin or viramidine. In one example, the additional agent active against HCV is interferon-alpha or pegylated interferon-alpha alone or in combination with ribavirin or viramidine. In another example, the active agent is interferon.

In other embodiments, provided are methods for preparing compounds of Formula (I). Details of the such methods can be found in Examples 1-43.

In one embodiment, provided is a method of preparing a compound of Formula (II) or a pharmaceutically acceptable salt thereof.

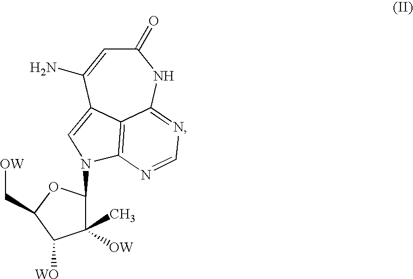

wherein W is optionally substituted C₁₋₆ alkyl(CO), said method comprising:

(a) reacting a compound of Formula (IIa)

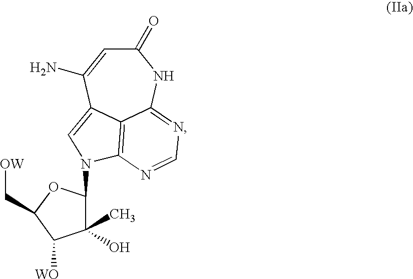

wherein W and W¹ are independently H or optionally substituted C₁₋₆ alkyl(CO), with optionally substituted C₁₋₆ alkyl(CO)OH and an amide coupling agent to form a compound of Formula (II); and

(b) optionally reacting a compound of Formula (II) with an acid to form a pharmaceutically acceptable salt thereof.

In some aspects one of W and W¹ is C₁₋₆ alkyl(CO). In other aspects both of W and W¹ are C₁₋₆ alkyl(CO).

In some aspects, the amide coupling agent is a carbodiimide coupling agent. In other aspects the coupling agent is N,N′-dicyclohexylcarbodiimde.

In some aspects the coupling reaction occurs in the presence of an a heteroaromatic amine such as dimethylaminopyridine.

In other the aspects the reaction occurs in a polar solvent. A suitable polar solvent is dimethylformamide.

In some aspects of the compound of Formula (II), W is CH₃(CO).

### Administration and Pharmaceutical Composition

The present invention provides novel compounds possessing antiviral activity, including Flaviviridae family viruses such as hepatitis C virus. The compounds of this invention inhibit viral replication by inhibiting the enzymes involved in replication, including RNA dependent RNA polymerase. They may also inhibit other enzymes utilized in the activity or proliferation of Flaviviridae viruses.

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors. The drug can be administered more than once a day, preferably once or twice a day.

Therapeutically effective amounts of compounds of the present invention may range from approximately 0.01 to 50 mg per kilogram body weight of the recipient per day; preferably about 0.01-25 mg/kg/day, more preferably from about 0.1 to 10 mg/kg/day. Thus, for administration to a 70 kg person, the dosage range would most preferably be about 7-70 mg per day.

This invention is not limited to any particular composition or pharmaceutical carrier, as such may vary. In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of this invention is inhalation.

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of the present invention in combination with at least one pharmaceutically acceptable excipient. Acceptable excipients are non-toxic, aid administration, and do not adversely affect the therapeutic benefit of the claimed compounds. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of a compound of the present invention based on the total formulation, with the balance being one or more suitable pharmaceutical excipients. Preferably, the compound is present at a level of about 1-80 wt %. Representative pharmaceutical formulations are described in the Formulation Examples section below.

Additionally, the present invention is directed to a pharmaceutical composition comprising a therapeutically effective amount of a compound of the present invention in combination with a therapeutically effective amount of another active agent against RNA-dependent RNA virus and, in particular, against HCV. Agents active against HCV include, but are not limited to, ribavirin, levovirin, viramidine, thymosin alpha-1, an inhibitor of HCV NS3 serine protease, or an inhibitor of inosine monophosphate dehydrognease, interferon-α, pegylated interferon-α (peginterferon-α), a combination of interferon-α and ribavirin, a combination of peginterferon-α and ribavirin, a combination of interferon-α and levovirin, and a combination of peginterferon-α and levovirin. Interferon-α includes, but is not limited to, recombinant interferon-α2a (such as ROFERON interferon available from Hoffman-LaRoche, Nutley, N.J.), interferon-α2b (such as Intron-A interferon available from Schering Corp., Kenilworth, N.J., USA), a consensus interferon, and a purified interferon-α product. For a discussion of ribavirin and its activity against HCV, see J. O, Saunders and S. A. Raybuck, “Inosine Monophosphate Dehydrogenase: Consideration of Structure, Kinetics and Therapeutic Potential,” Ann. Rep. Med. Chem., 35:201-210 (2000).

The agents active against hepatitis C virus also include agents that inhibit HCV proteases, HCV polymerase, HCV helicase, HCV NS4B protein, HCV entry, HCV assembly, HCV egress, HCV NS5A protein, and inosine 5′-monophosphate dehydrogenase. Other agents include nucleoside analogs for the treatment of an HCV infection. Still other compounds include those disclosed in WO 2004/014313 and WO 2004/014852 and in the references cited therein. The patent applications WO 2004/014313 and WO 2004/014852 are hereby incorporated by references in their entirety.

Specific antiviral agents include Omega IFN (BioMedicines Inc.), BILN-2061 (Boehringer Ingelheim), Summetrel (Endo Pharmaceuticals Holdings Inc.), Roferon A (F. Hoffman-La Roche), Pegasys (F. Hoffman-La Roche), Pegasys/Ribaravin (F. Hoffman-La Roche), CellCept (F. Hoffman-La Roche), Wellferon (GlaxoSmithKline), Albuferon-α (Human Genome Sciences Inc.), Levovirin (ICN Pharmaceuticals), IDN-6556 (Idun Pharmaceuticals), IP-501 (Indevus Pharmaceuticals), Actimmune (InterMune Inc.), Infergen A (InterMune Inc.), ISIS 14803 (ISIS Pharamceuticals Inc.), JTK-003 (Japan Tobacco Inc.), Pegasys/Ceplene (Maxim Pharmaceuticals), Ceplene (Maxim Pharmaceuticals), Civacir (Nabi Biopharmaceuticals Inc.), Intron A/Zadaxin (RegeneRx), Levovirin (Ribapharm Inc.), Viramidine (Ribapharm Inc.), Heptazyme (Ribozyme Pharmaceuticals), Intron A (Schering-Plough), PEG-Intron (Schering-Plough), Rebetron (Schering-Plough), Ribavirin (Schering-Plough), PEG-Intron/Ribavirin (Schering-Plough), Zadazim (SciClone), Rebif (Serono), IFN-β/EMZ701 (Transition Therapeutics), T67 (Tularik Inc.), VX-497 (Vertex Pharmaceuticals Inc.), VX-950/LY-570310 (Vertex Pharmaceuticals Inc.), Omniferon (Viragen Inc.), XTL-002 (XTL Biopharmaceuticals), SCH 503034 (Schering-Plough), isatoribine and its prodrugs ANA971 and ANA975 (Anadys), R1479 (Roche Biosciences), Valopicitabine (Idenix), NIM811 (Novartis), and Actilon (Coley Pharmaceuticals).

In some embodiments, the compositions and methods of the present invention contain a compound of the invention and interferon. In some aspects, the interferon is selected from the group consisting of interferon alpha 2B, pegylated interferon alpha, consensus interferon, interferon alpha 2A, and lymphoblastiod interferon tau.

In other embodiments the compositions and methods of the present invention contain a compound of the invention and a compound having anti-HCV activity is selected from the group consisting of interleukin 2, interleukin 6, interleukin 12, a compound that enhances the development of a type 1 helper T cell response, interfering RNA, anti-sense RNA, Imiquimod, ribavirin, an inosine 5′-monophospate dehydrogenase inhibitor, amantadine, and rimantadine.

In still other embodiments, the compound having anti-HCV activity is Ribavirin, levovirin, viramidine, thymosin alpha-1, an inhibitor of NS3 serine protease, and inhibitor of inosine monophosphate dehydrogenase, interferon-alpha, or pegylated interferon-alpha alone or in combination with Ribavirin or viramidine.

In another embodiments, the compound having anti-HCV activity is said agent active against HCV is interferon-alpha or pegylated interferon-alpha alone or in combination with Ribavirin or viramidine.

### EXAMPLES

In the examples below the following abbreviations have the indicated meanings. If an abbreviation is not defined, it has its generally accepted meaning.

| | |
|---|---|
| aq. = | aqueous |
| μL = | microliters |
| μM = | micromolar |
| NMR = | nuclear magnetic resonance |
| br = | broad |
| d = | doublet |
| δ = | chemical shift |
| ° C. = | degrees celcius |
| DCC = | N,N′-dicyclohexylcarbodiimide |
| dd = | doublet of doublets |
| DMEM = | Dulbeco's Modified Eagle's Medium |
| DMF = | N,N-dimethylformamide |
| DMSO = | dimethylsulfoxide |
| DTT = | dithiothreotol |
| EDTA = | ethylenediaminetetraacetic acid |
| EtOH = | ethanol |
| g = | gram |
| h or hr = | hours |
| HCV = | hepatitus C virus |
| HPLC = | high performance liquid chromatography |
| Hz = | hertz |
| IU = | International Units |
| IC₅₀ = | inhibitory concentration at 50% inhibition |
| J = | coupling constant (given in Hz unless otherwise |
| | indicated) |
| m = | multiplet |
| M = | molar |
| M + H⁺= | parent mass spectrum peak plus H⁺ |
| MeOH = | methanol |
| mg = | milligram |
| mL = | milliliter |
| mM = | millimolar |
| mmol = | millimole |
| MS = | mass spectrum |
| nm = | nanomolar |
| ng = | nanogram |
| ppm = | parts per million |
| HPLC = | high performance liquid chromatographY |
| s = | Singlet |
| t = | triplet |
| TEA = | triethylamine |
| TFA = | trifluoroacetic acid |
| wt % = | weight percent |

### Example 1

### Preparation of Hexanoic Acid 5-(9-amino-7-oxo-6,7-dihydro-2,3,5,6-tetraaza-benzo[cd]azulen-2-yl)-3,4-dihydroxy-4-methyl-tetrahydro-furan-2-ylmethyl Ester (Compound 101)

To a solution of 9-amino-2-(3,4-dihydroxy-5-hydroxymethyl-3-methyl-tetrahydro-furan-2-yl)-2,6-dihydro-2,3,5,6-tetraaza-benzo[cd]azulen-7-one (compound 100, prepared according to WO 2006/093987, published on Sep. 8, 2006, 100 mg, 0.288 mmol) in pyridine (2.9 mL) was added DMAP (52 mg, 0.432 mmol) and hexanoyl chloride (80.0 μL, 0.576 mmol) and the reaction was stirred at room temperature. The reaction was complete in 6 hours as was determined by QC-LCMS. The reaction mixture was concentrated in vacuo then re-dissolved in EtOAc and washed with 0.001M HCl. The organic layer was then dried over MgSO₄ and concentrated in vacuo. The crude reaction mixture was purified on reverse phase HPLC (0-100% buffer B over 30 minutes at 10 mL/min flow rate—Buffer A=H2O; Buffer B=ACN). One of the fractions yielded 25 mg (20%) of compound 101.

¹H NMR (DMSO-d₆): δ 10.08 (d, 1H, J=1.5 Hz), 8.32 (s, 1H), 7.78 (s, 1H), 6.77 (br s, 2H), 6.21 (s, 1H), 5.48 (d, 1H, J=7.2) 5.41 (s, 1H), 5.04 (d, 1H, J=1.5 Hz), 4.47-4.43 (m, 1H), 4.34-4.28 (m, 1H), 4.14-4.05 (m, 1H), 3.92-3.86 (m, 1H) 2.34 (t, 2H, J=7.8 Hz), 1.51 (m, 2H), 1.24 (m, 4H), 0.83 (t, 3H), 0.78 (s, 3H).

MS: m/z=446.2 (M+1)

### Example 2

### Preparation of Hexanoic Acid 5-(9-amino-7-oxo-6,7-dihydro-2,3,5,6-tetraaza-benzo[cd]azulen-2-yl)-3-hexanoyloxy-4-hydroxy-4-methyl-tetrahydro-furan-2-ylmethyl Ester (Compound 102)

To a solution of 9-amino-2-(3,4-dihydroxy-5-hydroxymethyl-3-methyl-tetrahydro-furan-2-yl)-2,6-dihydro-2,3,5,6-tetraaza-benzo[cd]azulen-7-one (compound 100, prepared according to WO 2006/093987, published on Sep. 8, 2006, 500 mg, 1.441 mmol) in pyridine (14.4 mL) was added (dimethylaminopyridine DMAP 263 mg, 2.161 mmol) and hexanoyl chloride (201 μL, 1.441 mmol) and the reaction was stirred at room temperature overnight. Reaction was monitored by QC-LCMS and showed a mixture of mono and di-acylated products. The reaction was quenched with MeOH, concentrated in vacuo and purified on Isco CombiFlash purification system utilizing a 40 g silica gel column and 0-20% MeOH gradient in DCM as the eluent over 30 minutes followed by a second purification on reverse phase HPLC (20-100% buffer B over 30 minutes at 10 mL/min flow rate—Buffer A=H₂O; Buffer B=ACN) to afford 40 mg (5%) of compound 102.

¹H NMR (DMSO-d₆): δ 10.15 (s, 1H), 8.33 (s, 1H), 7.89 (s, 1H), 6.83 (br s, 2H), 6.23 (s, 1H), 5.87 (s, 1H) 5.18 (d, 1H, J=7.8 Hz), 5.06 (s, 1H), 4.40-4.30 (m, 3H), 2.43 (t, 2H, J=7.5 Hz), 2.33 (t, 2H, J=7.5 Hz), 1.60-1.45 (m, 4H), 1.30-1.20 (m, 8H), 0.89-0.82 (m, 9H).

MS: m/z=544.3 (M+1)

### Example 3

### Preparation of Carbonic Acid 5-(9-amino-7-oxo-6,7-dihydro-2,3,5,6-tetraaza-benzo[cd]azulen-2-yl)-3,4-dihydroxy-4-methyl-tetrahydro-furan-2-ylmethyl Ester Pentyl Ester (Compound 103)

To a solution of 9-amino-2-(3,4-dihydroxy-5-hydroxymethyl-3-methyl-tetrahydro-furan-2-yl)-2,6-dihydro-2,3,5,6-tetraaza-benzo[cd]azulen-7-one (compound 100, prepared according to WO 2006/093987, published on Sep. 8, 2006, 125 mg, 0.360 mmol) in pyridine (2.4 mL) was added DMAP (65.9 mg, 0.540 mmol) and chloroformic acid n-amylester (78.2 μL, 0.540 mmol) and the reaction was stirred at room temperature overnight. The reaction stalled with 50% starting material determined by QC-HPLC. The reaction was quenched with MeOH, concentrated in vacuo and purified on Isco CombiFlash purification system utilizing a 4 g silica gel column and 0-20% MeOH gradient in DCM as the eluent over 20 minutes followed by a second purification on reverse phase HPLC (0-100% buffer B over 30 minutes at 10 mL/min flow rate—Buffer A=H₂O; Buffer B=ACN, acetonitrile) to afford 23 mg (14%) of compound 103.

¹H NMR (DMSO-d₆): δ 10.09 (s, 1H), 8.31 (s, 1H), 7.78 (s, 1H), 6.74 (br s, 2H), 6.19 (s, 1H), 5.53 (d, 1H, J=6.6 Hz) 5.44 (s, 1H), 5.05 (d, 1H, J=1.5 Hz), 4.48-4.44 (m, 2H), 4.15-4.05 (m, 3H), 3.88-3.83 (m, 1H), 1.58 (m, 2H), 1.27 (m, 4H), 0.85 (t, 3H, J=6.6 Hz), 0.76 (s, 3H).

MS: m/z=462.2 (M+1)

### Example 4

### Preparation of 2-amino-N-[2-(3,4-dihydroxy-5-hydroxymethyl-3-methyl-tetrahydro-furan-2-yl)-7-oxo-6,7-dihydro-2H-2,3,5,6-tetraaza-benzo[cd]azulen-9-yl]-acetamide (Compound 104)

### Step 1: 9-amino-2-(2,2-di-tert-butyl-7-hydroxy-7-methyl-tetrahydro-furo[3,2-d][1,3,2]dioxasilin-6-yl)-2,6-dihydro-2,3,5,6-tetraaza-benzo[cd]azulen-7-one

To a solution of 9-amino-2-(3,4-dihydroxy-5-hydroxymethyl-3-methyl-tetrahydro-furan-2-yl)-2,6-dihydro-2,3,5,6-tetraaza-benzo[cd]azulen-7-one (compound 100, prepared according to WO 2006/093987, published on Sep. 8, 2006, 500 mg, 1.441 mmol) in DMF (5.76 mL) was added imidazole followed by the dropwise addition of di-tert-butylsilyl bis(trifluoromethane sulfonate) under rapid stirring. The reaction mixture was stirred at room temperature for 3 hours then quenched with MeOH, concentrated in vacuo onto celite and purified on Isco CombiFlash purification system utilizing a 40 g silica gel column and 0-20% MeOH gradient in DCM as the eluent over 20 minutes to afford 450 mg (64%).

MS: m/z=488.2 (M+1)

### Step 2: {[2-(2,2-di-tert-butyl-7-hydroxy-7-methyl-tetrahydro-furo[3,2-d][1,3,2]dioxasilin-6-yl)-7-oxo-6,7-dihydro-2H-2,3,5,6-tetraaza-benzo[cd]azulen-9-ylcarbamoyl]-methyl}-carbamic Acid Benzyl Ester

To the product from Step 1 (100 mg, 0.205 mmol) in pyridine was added TMSCl (trimethylsilylchloride 26 μL, 0.205 mmol) and the mixture was allowed to stir for 1 hour. To this nucleoside solution at 0° C. was added a ˜1.38M solution Cbz-glycine acid chloride (1 mL, 1.38 mmol) which was made as follows. A 2M solution of oxalyl chloride (690 μL, 1.38 mmol) in DCM 10 mL was added to a solution of DMF (103 μl, 1.38 mmol) at 0° C. followed by the addition of pyridine (111 μl, 1.38 mmol). This solution was cooled to negative 20-25° C. and Cbz-gylcine (288 mg, 1.38 mmol) was added and the mixture was stirred at negative 20-25° C. for 2 hours. Prior to use, the Cbz-glycine acid chloride solution was concentrated to ˜1 mL (˜1.38M solution). This reaction procedure was repeated a second time on the same scale (100 mg starting nucleoside) and the two reactions were pooled, quenched with MeOH, concentrated in vacuo onto celite. The crude material was purified on Isco CombiFlash purification system utilizing a 40 g silica gel column and 0-10% MeOH gradient in DCM as the eluent over 20 minutes followed by purified on reverse phase HPLC (30-100% buffer B over 20 minutes at 20 mL/min flow rate—Buffer A=H2O; Buffer B=ACN) to afford 135 mg (˜48%) of slightly impure product.

MS: 679.2 (M+1)

### Step 3: {[2-(3,4-dihydroxy-5-hydroxymethyl-3-methyl-tetrahydro-furan-2-yl)-7-oxo-6,7-dihydro-2H-2,3,5,6-tetraaza-benzo[cd]azulen-9-ylcarbamoyl]-methyl}-carbamic Acid Benzyl Ester

To a solution of the product from Step 2 (135 mg, 0.199 mmol) in THF 1.9 mL at 0° C. was added TEA.3HF (32 μL, 0.199 mmol) and reaction was allowed to stir at 0° C. The reaction progress was monitored by QC-HPLC and was complete in 0.5 hours. Upon completion, the reaction was concentrated in vacuo and purified on reverse phase HPLC (30-100% buffer B over 20 minutes at 20 mL/min flow rate—Buffer A=H₂O; Buffer B=ACN) to afford 75 mg (70%) of a mixture of two products.

### Step 4: 2-amino-N-[2-(3,4-dihydroxy-5-hydroxymethyl-3-methyl-tetrahydro-furan-2-yl)-7-oxo-6,7-dihydro-2H-2,3,5,6-tetraaza-benzo[cd]azulen-9-yl]-acetamide (Compound 104)

To a solution of the product from Step 3 (75 mg, 0.139 mmol) in MeOH 5 mL containing 1% v/v acetic acid was added palladium on carbon (25 mg, 10% Pd by weight) and the mixture was maintained under a blanket of hydrogen via balloon (1 atmosphere). The reaction progress was monitored by QC-HPLC and was complete in 2 hours. The palladium was filtered off, the filtrate was concentrated in vacuo and purified on reverse phase HPLC (0-50% buffer B over 20 minutes at 20 mL/min flow rate—Buffer A=H₂O w/0.1% TFA; Buffer B=ACN w/0.1% TFA to afford 12 mg (17%) of compound 104 as the TFA salt after lyophilization. The compound was converted to the HCl salt by re-dissolving in 10 mL water containing 4 molar equivalence of HCL and lyophilizing a second time.

¹H NMR (DMSO-d₆): δ 10.84 (s, 1H), 9.93 (br s, 1H), 8.38 (s, 1H), 8.12 (br s, 3H), 7.95 (s, 1H), 6.56 (s, 1H), 6.15 (s, 1H), 5.35 (s, 1H), 5.20 (d, 1H, J=6.9 Hz), 4.98 (br s, 1H), 3.93-3.65 (m, 6H), 0.78 (s, 3H).

MS: 405.1 (M+1)

### Example 5

### Preparation of Isobutyric Acid 5-(9-amino-7-oxo-6,7-dihydro-2,3,5,6-tetraaza-benzo[cd]azulen-2-yl)-3,4-dihydroxy-4-methyl-tetrahydro-furan-2-ylmethyl Ester (Compound 105) and isobutyric acid 5-(9-amino-7-oxo-6,7-dihydro-2,3,5,6-tetraaza-benzo[cd]azulen-2-yl)-4-hydroxy-3-isobutyryloxy-4-methyl-tetrahydro-furan-2-ylmethyl Ester (Compound 134)

To a solution of 9-amino-2-(3,4-dihydroxy-5-hydroxymethyl-3-methyl-tetrahydro-furan-2-yl)-2,6-dihydro-2,3,5,6-tetraaza-benzo[cd]azulen-7-one (compound 100, prepared according to WO 2006/093987, published on Sep. 8, 2006, 250 mg, 0.720 mmol) in pyridine (7.3 mL) was added resin bound DMAP (118 mg, 1.52 mmol/g resin) and the solution was cooled to 0° C. Isobutyryl chloride (137 μL, 1.30 mmol) was added to the mixture in 22.8 μl aliquots every hour for 6 hours. After 6.5 hours, the reaction was quenched with the addition of silica gel, concentrated in vacuo and purified on Isco CombiFlash purification system utilizing a 40 g silica gel column and 0-20% MeOH gradient in DCM as the eluent over 20 minutes followed by a second purification on reverse phase HPLC (0-80% buffer B over 30 minutes at 20 mL/min flow rate—Buffer A=H2O; Buffer B=ACN) to afford 45 mg (15%) of compound 105, 15 mg (4%) of compound 134 and other side products.

Compound 105:

¹H NMR (DMSO-d₆): δ 10.08 (s, 1H), 8.32 (s, 1H), 7.78 (s, 1H), 6.78 (s, 2H), 6.21 (s, 1H), 5.50 (d, 1H, J=6.6 Hz) 5.42 (s, 1H), 5.04 (d, 1H, J=1.8 Hz), 4.48-4.42 (m, 1H), 4.37-4.30 (m, 1H), 4.14-4.06 (m, 1H), 3.89-3.83 (m, 1H), 2.6 (m, 1H), 1.1 (d, 3H, J=3 Hz), 1.08 (d, 3H, J=3 Hz), 0.77 (s, 3H).

MS: m/z=418.2 (M+1)

Compound 134:

¹H NMR (DMSO-d₆): δ 10.13 (s, 1H), 8.33 (s, 1H), 7.90 (s, 1H), 6.82 (s, 2H), 6.24 (s, 1H), 5.90 (s, 1H) 5.16 (d, 1H, J=8.1 Hz), 5.06 (s, 1H), 4.36 (m, 3H), 2.68 (m, 1H), 2.54 (m, 1H), 1.15 (d, 6H, J=6.9 Hz), 1.09 (d, 3H, J=3 Hz), 1.09 (d, 3H, J=3 Hz), 0.82 (s, 3H).

MS: m/z=488.3 (M+1)

### Example 6

### Preparation of Isobutyric Acid 6-(9-amino-7-oxo-6,7-dihydro-2,3,5,6-tetraaza-benzo[cd]azulen-2-yl)-6a-methyl-2-oxo-tetrahydro-furo[3,4-d][1,3]dioxol-4-ylmethyl Ester (Compound 106)

To a solution of compound 105 (Example 5, 30 mg, 0.072 mmol) in DMF (0.719 mL) was added CDI (35 mg, 0.216 mmol) and the reaction was stirred at room temp for 3 hours. The crude product was purified on reverse phase HPLC (30-100% buffer B over 30 minutes at 20 mL/min flow rate—Buffer A=H2O; Buffer B=ACN) to afford 23 mg (72%) of compound 106.

¹H NMR (DMSO-d₆): δ 10.19 (d, 1H, J=1.5 Hz), 8.36 (s, 1H), 7.93 (s, 1H), 6.76 (br s, 2H), 6.72 (s, 1H), 5.07 (d, 1H, J=1.5 Hz) 5.04 (d, 1H, J=4.5 Hz), 4.69-4.64 (m, 1H), 4.44-4.36 (m, 2H), 2.60 (m, 1H), 1.22 (s, 3H), 1.12 (d, 6H, J=6.9 Hz).

MS: m/z=440.2 (M+1)

### Example 7

### Preparation of Acetic Acid 5-(9-amino-7-oxo-6,7-dihydro-2,3,5,6-tetraaza-benzo[cd]azulen-2-yl)-3,4-dihydroxy-4-methyl-tetrahydro-furan-2-ylmethyl Ester (Compound 107) and acetic acid 3-acetoxy-5-(9-amino-7-oxo-6,7-dihydro-2,3,5,6-tetraaza-benzo[cd]azulen-2-yl)-4-hydroxy-4-methyl-tetrahydro-furan-2-ylmethyl Ester (Compound 135)

To a solution of 9-amino-2-(3,4-dihydroxy-5-hydroxymethyl-3-methyl-tetrahydro-furan-2-yl)-2,6-dihydro-2,3,5,6-tetraaza-benzo[cd]azulen-7-one (compound 100, prepared according to WO 2006/093987, published on Sep. 8, 2006, 250 mg, 0.720 mmol) in DMF (0.72 mL) was added DMAP (132 mg, 1.08 mmol) and acetyl chloride (102 μL, 1.44 mmol) and the reaction was stirred at room temperature for 1 hour. The crude product mixture was purified on reverse phase HPLC (0-60% buffer B over 30 minutes at 20 mL/min flow rate—Buffer A=H2O; Buffer B=ACN) to afford 40 mg (14%) of and 55 mg (18%) of compound 107.

Compound 107:

¹H NMR (DMSO-d₆): δ 10.10 (d, 1H, J=1.8 Hz), 8.32 (s, 1H), 7.78 (s, 1H), 6.77 (br s, 2H), 6.21 (s, 1H), 5.50 (d, 1H, J=6.9 Hz) 5.42 (s, 1H), 5.04 (d, 1H, J=1.8 Hz), 4.46-4.40 (m, 1H), 4.36-4.28 (m, 1H), 4.20-4.06 (m, 1H), 3.92-3.85 (m, 1H), 2.05 (s, 3H), 0.78 (s, 3H).

MS: m/z=390.2 (M+1)

Compound 135:

¹H NMR (DMSO-d₆): δ 10.14 (d, 1H, J=1.5 Hz), 8.33 (s, 1H), 7.89 (s, 1H), 6.83 (br s, 2H), 6.23 (s, 1H), 5.87 (s, 1H) 5.16 (d, 1H, J=7.8 Hz), 5.06 (d, 1H, J=1.5 Hz), 4.40-4.30 (m, 3H), 2.14 (s, 3H), 2.05 (s, 3H), 0.83 (s, 3H).

MS: m/z=432.2 (M+1)

### Example 8

### Preparation of Acetic Acid 6-(9-amino-7-oxo-6,7-dihydro-2,3,5,6-tetraaza-benzo[cd]azulen-2-yl)-6a-methyl-2-oxo-tetrahydro-furo[3,4-d][1,3]dioxol-4-ylmethylester (Compound 108)

To a solution of compound 135 (Example 7, 40 mg, 0.103 mmol) in DMF (1 mL) was added CDI (50 mg, 0.308 mmol) and the mixture was allowed to stir at room temp for 3.5 hours. The crude reaction product was purified on reverse phase HPLC (0-80% buffer B over 30 minutes at 20 mL/min flow rate—Buffer A=H2O; Buffer B=ACN) to afford 30 mg (70%) of compound 108.

¹H NMR (DMSO-d₆): δ 10.19 (s, 1H), 8.36 (s, 1H), 7.92 (s, 1H), 6.78 (br s, 2H), 6.72 (s, 1H), 5.08 (d, 1H, J=1.5 Hz) 5.04 (d, 1H, J=4.2 Hz), 4.69-4.64 (m, 1H), 4.44-4.36 (m, 2H), 2.08 (s, 3H), 1.22 (s, 3H).

MS: 416.2 (M+1)

### Example 9

### Preparation of Isobutyric Acid 2-(3,4-dihydroxy-5-hydroxymethyl-3-methyl-tetrahydro-furan-2-yl)-7-oxo-6,7-dihydro-2H-2,3,5,6-tetraaza-benzo[cd]azulen-9-ylcarbamoyloxymethyl Ester (Compound 109)

### Step 1: Thiocarbonic Acid O-Chloromethyl Ester S-Ethyl Ester

To a suspension of sodium ethanethiolate (4.21 g, 0.05 mol) in ether (100 mL) at −78° C. was added a solution of chloromethyl chloroformate (4.40 mL, 0.05 mol) in ether (50 mL) dropwise via addition funnel over 1 hour. Reaction was stirred at −78° C. for an additional hour then at room temperature overnight. The salts were removed by filtration and the organic layer was washed with water, dried over Na₂SO₄ and concentrated in vacuo. The crude product was used in Step 2 without further purification.

### Step 2: Isobutyric Acid Ethylsulfanylcarbonyloxymethyl Ester

The crude product from Step 1 (2.05 g, 13.3 mmol) was added to a suspension of cesium isobutyrate (3.3 g, 14.6 mmol) in DMF (25 mL) and the mixture was allowed to stir overnight. The reaction was concentrated in vacuo, re-dissolved in DCM and washed with saturated aqueous sodium bicarbonate solution followed by water. The organic layer was dried over Na₂SO₄ and concentrated in vacuo. The product was purified by distillation under vacuum.

### Step 3: Isobutryloxymethyl Carbonochloridate

The isobutryloxymethyl carbonochloridate was synthesized from the product of Step 2 utilizing the general procedure for making acyloxymethyl carbonochloridates as described in the literature (Synthesis 1990, 1159-1166).

### Step 4: Isobutyric Acid 2-(2,2-di-tert-butyl-7-hydroxy-7-methyl-tetrahydro-furo[3,2-d][1,3,2]dioxasilin-6-yl)-7-oxo-6,7-dihydro-2H-2,3,5,6-tetraaza-benzo[cd]azulen-9-ylcarbamoyloxymethyl Ester

To the product of Step 1, Example 4 (100 mg, 0.205 mmol) and DMAP (37.5 mg, 0.308 mmol) in pyridine at 0° C. was added the product of Step 3 (158 μL, 0.821 mmol) and the reaction was allowed to warm to room temperature. The reaction progress was monitored by QC-LCMS and after 1 hour was quench with MeOH, concentrated in vacuo and purified on Isco CombiFlash purification system utilizing a 12 g silica gel column and 0-10% MeOH gradient in DCM as the eluent over 20 minutes to afford 47 mg (36%).

### Step 5: Isobutyric Acid 2-(3,4-dihydroxy-5-hydroxymethyl-3-methyl-tetrahydro-furan-2-yl)-7-oxo-6,7-dihydro-2H-2,3,5,6-tetraaza-benzo[cd]azulen-9-ylcarbamoyloxymethyl Ester (Compound 109)

To a solution of the product from Step 4 (47 mg, 0.074 mmol) in THF (0.75 mL) was added TEA.3HF (36.4 μL, 0.223 mmol) at 0° C. and the reaction was allowed to warm to room temperature. The reaction progress was monitored by QC-LCMS and was determined complete in 30 minutes. The crude product was purified on reverse phase HPLC (0-100% buffer B over 20 minutes at 20 mL/min flow rate—Buffer A=H2O; Buffer B=ACN) to afford 15 mg (41%) of compound 109.

¹H NMR (DMSO-d₆): δ 10.77 (s, 1H), 9.83 (s, 1H), 8.36 (s, 1H), 7.95 (s, 1H), 6.55 (d, 1H, J=1.5 Hz), 6.18 (s, 1H), 5.80 (s, 2H), 5.29 (s, 1H) 5.17 (m, 1H), 4.90 (t, 1H, J=5.1 Hz), 3.94-3.88 (m, 2H), 3.84-3.64 (m, 2H), 2.64 (m, 1H), 1.14 (d, 3H, J=6.9 Hz), 1.13 (d, 3H, J=6.9 Hz), 0.77 (s, 1H).

MS: m/z=492.3 (M+1)

### Example 10

### Preparation of 2-amino-3-methyl-butyric acid 4-hydroxy-2-hydroxymethyl-5-(9-isobutyryloxymethoxycarbonylamino-7-oxo-6,7-dihydro-2,3,5,6-tetraaza-benzo[cd]azulen-2-yl)-4-methyl-tetrahydro-furan-3-yl Ester (Compound 110)

### Step 1: 9-amino-2-[5-(tert-butyl-dimethyl-silanyloxymethyl)-3,4-dihydroxy-3-methyl-tetrahydro-furan-2-yl]-2,6-dihydro-2,3,5,6-tetraaza-benzo[cd]azulen-7-one

To a solution of 9-amino-2-(3,4-dihydroxy-5-hydroxymethyl-3-methyl-tetrahydro-furan-2-yl)-2,6-dihydro-2,3,5,6-tetraaza-benzo[cd]azulen-7-one (compound 100, prepared according to WO 2006/093987, published on Sep. 8, 2006, 550 mg, 1.59 mmol) in DMF (16 mL) was added imidazole (323 mg, 4.76 mmol) followed by the dropwise addition of tert-butyldimethylsilyl chloride in DMF (3 mL) under rapid stirring. The reaction was stirred at room temperature and monitored by QC-HPLC. After 1 hour, the reaction was quenched with MeOH, concentrated in vacuo onto celite and purified on Isco CombiFlash purification system utilizing a 40 g silica gel column and 0-30% MeOH gradient in DCM as the eluent over 20 minutes to afford 300 mg (41%).

MS: m/z=462.2 (M+1)

### Step 2: 2-benzyloxycarbonylamino-3-methyl-butyric acid 5-(9-amino-7-oxo-6,7-dihydro-2,3,5,6-tetraaza-benzo[cd]azulen-2-yl)-2-(tert-butyl-dimethyl-silanyloxymethyl)-4-hydroxy-4-methyl-tetrahydro-furan-3-yl Ester

A solution of the product from Step 1 (120 mg, 0.260 mmol) in DMF (2.6 mL) was added directly to a dry mixture of DCC (107 mg, 0.521 mmol), carbobenzyloxy-L-valine (131 mg, 0.521 mmol) and DMAP (63.5 mg, 0.521 mmol) and the mixture was stirred at room temperature overnight. The reaction was quenched with MeOH, concentrated in vacuo onto celite and purified on Isco CombiFlash purification system utilizing a 40 g silica gel column and 0-20% MeOH gradient in DCM as the eluent over 20 minutes to afford 110 mg (61%).

MS: m/z=595.3 (M+1)

### Step 3: 2-benzyloxycarbonylamino-3-methyl-butyric acid 2-(tert-butyl-dimethyl-silanyloxymethyl)-4-hydroxy-5-(9-isobutyryloxymethoxycarbonylamino-7-oxo-6,7-dihydro-2,3,5,6-tetraaza-benzo[cd]azulen-2-yl)-4-methyl-tetrahydro-furan-3-yl Ester

To a solution of the product from Step 2 (110 mg, 0.158 mmol) in pyridine (1.6 mL) with DMAP (29.0 mg, 0.237 mmol) was added the product of Step 3, Example 13 (114 μL, 0.633 mmol) at 0° C. and the reaction was allowed to warm to room temperature. The reaction progress was monitored by QC-HPLC. The reaction was quenched with MeOH, concentrated in vacuo onto celite and purified on Isco CombiFlash purification system utilizing a 12 g silica gel column and 0-20% MeOH gradient in DCM as the eluent over 20 minutes to afford 120 mg (90%) of slightly impure material.

MS: m/z=839.3 (M+1)

### Step 4: 2-benzyloxycarbonylamino-3-methyl-butyric acid 4-hydroxy-2-hydroxymethyl-5-(9-isobutyryloxymethoxycarbonylamino-7-oxo-6,7-dihydro-2,3,5,6-tetraaza-benzo[cd]azulen-2-yl)-4-methyl-tetrahydro-furan-3-yl Ester

To a solution of the product from Step 3 (50 mg, 0.0596 mmol) in THF (0.6 mL) was added TEA.3HF (10 μL, 0.0596 mmol) at 0° C. The mixture was allowed to warm to room temperature and monitored by QC-HPLC. After 1 hour a second 10 μL of TEA.3HF was added and continued monitoring via QC-HPCL. Reaction was complete after 4.5 hours. The crude mixture was purified by reverse phase HPLC (20-100% buffer B over 20 minutes at 20 mL/min flow rate—Buffer A=H2O; Buffer B=ACN) to afford 30 mg (70%) of the desired product.

MS: 725.2 (M+1)

### Step 5: 2-amino-3-methyl-butyric acid 4-hydroxy-2-hydroxymethyl-5-(9-isobutyryloxymethoxycarbonylamino-7-oxo-6,7-dihydro-2,3,5,6-tetraaza-benzo[cd]azulen-2-yl)-4-methyl-tetrahydro-furan-3-yl Ester (compound 110)

To a solution of the product from Step 4 (20 mg, 0.028 mmol) in MeOH containing 1% AcOH was added Pd/C (10 mg, 10% Palladium by weight) and the mixture was maintained under a blanket of hydrogen via balloon (1 atmosphere). The reaction progress was monitored by QC-HPLC and was complete in 2.5 hours. The palladium was filtered off, the filtrate was concentrated in vacuo and purified on reverse phase HPLC (0-100% buffer B over 20 minutes at 20 mL/min flow rate—Buffer A=H2O w/0.1% TFA; Buffer B=ACN w/0.1% TFA to afford 8 mg (41%) of compound 110 as the TFA salt.

¹H NMR (DMSO-d₆): δ 10.82 (s, 1H), 9.85 (s, 1H), 8.38 (s, 1H), 8.34 (br s, 3H), 8.05 (s, 1H), 6.58 (d, 1H, J=1.5 Hz), 6.24 (s, 1H), 5.81 (s, 2H) 5.76 (br s, 1H), 5.26 (d, 1H, J=8.1 Hz) 5.19 (br s, 1H), 4.21 (m, 1H), 4.08 (m, 1H), 3.80-3.60 (m, 1H), 2.63 (m, 1H), 2.26 (m, 1H), 1.12 (d, 6H, J=6.9 Hz), 1.01 (m, 6H), 0.92 (s, 3H).

MS: 591.2 (M+1)

### Example 11

### Preparation of Isobutyric Acid 5-(9-acetoxymethoxycarbonylamino-7-oxo-6,7-dihydro-2,3,5,6-tetraaza-benzo[cd]azulen-2-yl)-4-hydroxy-2-isobutyryloxymethyl-4-methyl-tetrahydro-furan-3-yl Ester (Compound 111)

Into a solution of compound 134 (Example 5, 341 mg. 0.7 mmol) in anhydrous pyridine (6 mL) was added TMSCl (89 μL, 0.7 mmol) and the resulting mixture stirred at room temperature for 0.5 h. The mixture was then cooled to 0° C. and acetyloxymethyl chloroformate (0.32 g, 2.1 mmol) was added. After 40 min. stirring at 0° C. the reaction was quenched with MeOH, filtered and the filtrate concentrated. The residue was purified by column chromatography on silica gel using 0-10% gradient of MeOH in CH₂ Cl₂ to yield the target compound as a pale yellow solid (257 mg, 61%).

¹H NMR (DMSO-d₆): δ 10.83 (s, 1H), 9.90 (s, 1H), 8.38 (s, 1H), 7.92 (s, 1H), 6.64 (d, 1H, J=1.5 Hz), 6.25 (s, 1H), 5.90 (s, 1H), 5.79 (2 apparent d, 2H), 5.23 (d, 1H, J=8.7 Hz), 4.27-4.22 (3m, 3H), 2.65 (heptet, 1H, J=6.9 Hz), 2.11 (s, 3H), 1.14 (d, 6H, J=6.9 Hz), 1.11 (d, 3H, J=6.9 Hz), 1.04 (d, 3H, J=6.9 Hz), 0.87 (s, 3H, J=6.9 Hz).

MS: m/z=604.2 (M+1).

### Example 12

### Preparation of Isobutyric Acid 2-(4-acetoxy-3-hydroxy-5-hydroxymethyl-3-methyl-tetrahydro-furan-2-yl)-7-oxo-6,7-dihydro-2H-2,3,5,6-tetraaza-benzo[cd]azulen-9-ylcarbamoyloxymethyl Ester (Compound 112)

### Step 1: Acetic Acid 5-(9-amino-7-oxo-6,7-dihydro-2,3,5,6-tetraaza-benzo[cd]azulen-2-yl)-2-(tert-butyl-dimethyl-silanyloxymethyl)-4-hydroxy-4-methyl-tetrahydro-furan-3-yl Ester

9-Amino-2-[5-(tert-butyl-dimethyl-silanyloxymethyl)-3,4-dihydroxy-3-methyl-tetrahydro-furan-2-yl]-2,6-dihydro-2,3,5,6-tetraaza-benzo[cd]azulen-7-one (Example 10, Step 1, 470 mg, 1.0 mmol) was added into a mixture of DCC (416 mg, 2.0 mmol), DMAP (24 mg, 0.2 mmol) and acetic acid (117 μL, 2.0 mmol) in anhydrous DMF (10 mL). After an overnight stirring at room temperature the reaction was quenched with MeOH and white solid filtered off. The evaporated residue was triturated with MeOH, filtered and evaporated. Silica gel column chromatography with CH₂Cl₂/MeOH (gradient 0-10% MeOH)+0.5% pyridine yielded the target compound as pale-yellow foam (390 mg, 78%).

¹H NMR (DMSO-d₆): δ 10.21 (d, 1H, J=1.5 Hz), 8.34 (s, 1H), 7.87 (s, 1H), 6.84 (br, 2H), 6.21 (s, 1H), 5.77 (s, 1H), 5.09 (d, 1H, J=8.8 Hz), 5.05 (d, 1H, J=1.7 Hz), 4.13 (ddd, 1H, J=4.1, 6.8 and 8.5 Hz), 3.98 (dd, 1H, J=6.9 and 11.4 Hz), 3.84 (dd, 1H, J=4.1 and 11.4 Hz), 2.11 (s, 3H), 0.85 (s, 9H), 0.81 (s, 3H), 0.05 (s, 3H), 0.04 (s, 3H).

MS: m/z=504.2 (M+1).

### Step 2: Isobutyric Acid 2-(4-acetoxy-3-hydroxy-5-hydroxymethyl-3-methyl-tetrahydro-furan-2-yl)-7-oxo-6,7-dihydro-2H-2,3,5,6-tetraaza-benzo[cd]azulen-9-ylcarbamoyloxymethyl Ester

To an ice-cold solution of the product from Step 1 (390 mg, 0.77 mmol) and DMAP (19 mg, 0.15 mmol) in anhydrous pyridine (7 mL) was added isobutyryoxymethyl chloroformate (0.41 g, 2.3 mmol). Reaction mixture was stirred at 0° C. for 50 min then quenched with MeOH and evaporated. Purification on a silica gel column with CH₂Cl₂/MeOH (gradient 0-10% MeOH)+0.5% pyridine yielded the target compound as pale-yellow foam (367 mg, 74%).

¹H NMR (CDCl₃): δ 10.8 (br, 1H), 8.20 (br, 1H), 7.43 (s, 1H), 6.89 (d, 1H, J=1.2 Hz), 6.81 (s, 1H), 6.22 (s, 1H), 5.88 (d, 1H, J=12.3 Hz), 5.86 (d, 1H, J=12.3 Hz), 5.19 (d, 1H, J=5.9 Hz), 5.12 (br, 1H), 4.26 (m, 1H), 4.03 (dd, 1H, J=11.4 and 3.2 Hz), 3.95 (dd, 1H, J=11.4 and 3.8 Hz), 2.65 (heptet, 1H, J=6.9 Hz), 2.23 (s, 3H), 1.23 (d, 3H, J=7.0 Hz), 1.22 (d, 3H, J=7.0 Hz), 0.97 (s, 9H), 0.17 (s, 3H), 0.16 (s, 3H).

MS: m/z=648.3 (M+1).

### Step 3: Isobutyric Acid 2-(4-acetoxy-3-hydroxy-5-hydroxymethyl-3-methyl-tetrahydro-furan-2-yl)-7-oxo-6,7-dihydro-2H-2,3,5,6-tetraaza-benzo[cd]azulen-9-ylcarbamoyloxy-methyl Ester (Compound 112)

To a solution of compound from Step 2 (0.33 g, 0.5 mmol) in THF (5 mL) was added Et₃N.3HF (0.24 mL, 1.5 mmol) and the resulting mixture stirred overnight at room temperature. The reaction was quenched with silica and evaporated to dryness. Purification on a silica gel column with EtOAc as the eluent yielded 208 mg (78%) of the target compound.

¹H NMR (CD₃CN): δ 11.2 (br, 1H), 8.12 (s, 1H), 7.58 (s, 1H), 7.54 (s, 1H), 6.35 (d, 1H, J=1.5 Hz), 6.10 (s, 1H), 5.79 (s, 2H), 5.21 (s, 1H), 4.20 (m, 1H), 4.13 (m, 1H), 4.06-3.98 (m, 2H), 3.82 (m, 1H), 2.68 (heptet, 1H, J=7.1 Hz), 2.18 (s, 3H), 1.21 (d, 6H, J=6.9 Hz), 0.97 (s, 3H).

MS: m/z=534.7 (M+1).

### Example 13

### Preparation of Hexanoic Acid 5-(9-amino-7-oxo-6,7-dihydro-2,3,5,6-tetraaza-benzo[cd]azulen-2-yl)-4-hydroxy-2-hydroxymethyl-4-methyl-tetrahydro-furan-3-yl Ester (Compound 113)

### Step 1: 9-amino-2-[5-(tert-butyl-diphenyl-silanyloxymethyl)-3,4-dihydroxy-3-methyl-tetrahydro-furan-2-yl]-2,6-dihydro-2,3,5,6-tetraaza-benzo[cd]azulen-7-one

9-Amino-2-(3,4-dihydroxy-5-hydroxymethyl-3-methyl-tetrahydro-furan-2-yl)-2,6-dihydro-2,3,5,6-tetraaza-benzo[cd]azulen-7-one (compound 100, prepared according to WO 2006/093987, published on Sep. 8, 2006, 0.3 g, 0.86 mmol) was dissolved in anhydrous DMF (15 mL). To this solution, imidazole (0.5 g, 3.44 mmol) and TBDPSCl (0.77 mL, 3.44 mol) were added under argon. After stirring for overnight at room temperature, reaction was quenched with anhydrous EtOH (0.8 mL). The solvents were evaporated. Residue was purified by ISCO combiflash on silica gel column with MeOH/CH₂Cl₂ (0 to 30% gradient for 30 min) as the eluents to yield 560 mg (50%) of the target compound.

MS: 586.2 (M+1).

### Step 2: Hexanoic Acid 5-(9-amino-7-oxo-6,7-dihydro-2,3,5,6-tetraaza-benzo[cd]azulen-2-yl)-2-(tert-butyl-diphenyl-silanyloxymethyl)-4-hydroxy-4-methyl-tetrahydro-furan-3-yl Ester

The product from Step 1 (0.2 g, 0.34 mmol) was dissolved in anhydrous pyridine (10 mL), and then cooled to 0 to 5° C. (ice/water bath). DMAP (0.083 g, 0.68 mmol) and hexanoyl chloride (92 μL, 0.68 mmol) were added under argon. After stirring for 1 h at room temperature, additional DMAP (0.083 g, 0.68 mmol) and hexanoyl chloride (92 μL, 0.68 mmol) were added. After stirring for additional 1 h at room temperature, reaction mixture was quenched with anhydrous EtOH (0.8 mL). The solvents were evaporated. The residue was purified by ISCO combiflash on silica gel column with MeOH/CH₂Cl₂ (0 to 15% gradient for 30 min) as the eluents to yield 120 mg (52%) of the target compound.

MS: 684.3 (M+1).

### Step 3: Hexanoic Acid 5-(9-amino-7-oxo-6,7-dihydro-2,3,5,6-tetraaza-benzo[cd]azulen-2-yl)-4-hydroxy-2-hydroxymethyl-4-methyl-tetrahydro-furan-3-yl Ester (Compound 113)

The product from Step 2 (0.1 g, 0.15 mmol) was dissolved in anhydrous THF (5 mL). TBAF (290 μL, 0.3 mmol; 1M in THF) was added to this solution and the resulting mixture was stirred at room temperature for 4 h. The reaction mixture was diluted with MeOH (5 mL) and concentrated in vacuo. The solvents were evaporated. Residue was purified by ISCO combiflash on silica gel column with MeOH/CH₂Cl₂ (0 to 20% gradient for 30 min) as the eluents to yield 41 mg of the title compound.

¹H NMR (DMSO-d₆) δ 10.09 (s, 1H), 8.3 (s, 1H), 7.93 (s, 1H), 6.76 (bs, 2H), 6.2 (s, 1H), 5.7 (s, 1H), 5.09 (s, 1H), 5.05 (d, 1H, J=11.4), 4.97 (t, 1H), 4.14 (m, 1H), 3.69 (m, 1H), 2.39 (t, 2H, J=6.2), 1.55 (t, 2H, J=6.2), 1.26 (m, 4H), 0.9-0.86 (m, 6H).

MS (M+1): 446.3

### Example 14

### Preparation of Acetic Acid 5-(9-amino-7-oxo-6,7-dihydro-2,3,5,6-tetraaza-benzo[cd]azulen-2-yl)-4-hydroxy-2-hydroxymethyl-4-methyl-tetrahydro-furan-3-yl Ester (Compound 114)

### Step 1: Acetic Acid 5-(9-amino-7-oxo-6,7-dihydro-2,3,5,6-tetraaza-benzo[cd]azulen-2-yl)-2-(tert-butyl-diphenyl-silanyloxymethyl)-4-hydroxy-4-methyl-tetrahydro-furan-3-yl Ester

To a solution of the product from Example 13, Step 1 (170 mg, 0.29 mmol) in anhydrous pyridine (10 mL), and then cooled to 0 to 5° C. (ice/water bath). DMAP (0.071 g, 0.58 mmol) and acetyl chloride (46 μL1, 0.58 mmol) were added under argon. After stirring for 2 h at room temperature DMAP (0.071 g, 0.58 mmol) and acetyl chloride (46 μL, 0.58 mmol)were added. After stirring for next 2 h at room temperature, reaction mixture was quenched with anhydrous EtOH (0.8 mL). The solvents were evaporated up to dryness. Residue was purified by ISCO combiflash on silica gel column with MeOH/CH₂Cl₂ (0 to 15% gradient for 30 min) as the eluents to yield 101 mg of the target compound.

### Step 2: Acetic Acid 5-(9-amino-7-oxo-6,7-dihydro-2,3,5,6-tetraaza-benzo[cd]azulen-2-yl)-4-hydroxy-2-hydroxymethyl-4-methyl-tetrahydro-furan-3-yl Ester (Compound 114)

To a solution of the product from Step 1 (0.1 g, 0.16 mmol) in anhydrous THF (7 mL), was added TBAF (320 μL, 0.32 mmol; 1M in THF). The resulting mixture was stirred at room temperature for 5 h. the mixture was then diluted with MeOH (5 mL) and concentrated in vacuo. The solvents were evaporated. The residue was purified by ISCO combiflash on silica gel column with MeOH/CH₂Cl₂ (0 to 20% gradient for 30 min) as the eluents to yield 41 mg of the title compound.

¹H NMR (DMSO-d₆) δ 10.09 (s, 1H), 8.3 (s, 1H), 7.93 (s, 1H), 6.76 (bs, 2H), 6.2 (s, 1H), 5.7 (s, 1H), 5.08 (s, 1H), 5.05 (s, 1H), 5.0 (m, 3H), 4.15 (m, 1H), 3.72 (m, 2H), 2.26 (s, 3H), 0.81 (s, 3H).

MS (M+1): 390.2

### Example 15

### Preparation of Isobutyric Acid 5-(9-amino-7-oxo-6,7-dihydro-2,3,5,6-tetraaza-benzo[cd]azulen-2-yl)-4-hydroxy-2-hydroxymethyl-4-methyl-tetrahydro-furan-3-yl Ester (Compound 115)

### Step 1: Isobutyric Acid 5-(9-amino-7-oxo-6,7-dihydro-2,3,5,6-tetraaza-benzo[cd]azulen-2-yl)-2-(tert-butyl-diphenyl-silanyloxymethyl)-4-hydroxy-4-methyl-tetrahydro-furan-3-yl Ester

A solution of the product from Example 13, Step 1 (200 mg, 0.34 mmol) in anhydrous pyridine (10 mL) was cooled to 0 to 5° C. (ice/water bath). DMAP (0.083 g, 0.68 mmol) and isobutyryl chloride (73 μl, 0.68 mmol) were added under argon. After stirring for 1.5 h at room temperature, DMAP (0.030 g, 0.24 mmol) and isobutyryl chloride (31 μL, 0.29 mmol) were added. After stirring for additional 2 h at room temperature, reaction mixture was quenched with anhydrous EtOH (0.5 mL). The solvents were evaporated. The residue was purified by ISCO combiflash on silica gel column with MeOH/CH₂Cl₂ (0 to 15% gradient for 35 min) as the eluents to yield 87 mg of the target compound.

¹H NMR (DMSO-d₆) δ 10.12 (s, 1H), 8.3 (s, 1H), 7.84 (s, 1H), 7.61-7.31 (m, 10H), 6.72 (bs, 2H), 6.23 (s, 1H), 5.8 (s, 1H), 5.08-5.03 (m, 2H), 4.30-4.27 (m, 1H), 4.13-4.07 (m, 1H), 3.83-3.78 (m, 1H), 2.6-2.52 (m, 1H), 1.06, 1.02 (2×d, 6H, J=5.6), 0.78 (s, 3H).

### Step 2: Isobutyric Acid 5-(9-amino-7-oxo-6,7-dihydro-2,3,5,6-tetraaza-benzo[cd]azulen-2-yl)-4-hydroxy-2-hydroxymethyl-4-methyl-tetrahydro-furan-3-yl Ester (Compound 115)

To a solution of the product from Step 1 (0.087 g, 0.13 mmol) in anhydrous THF (7 mL), TBAF (260 μL, 0.26 mmol; 1M in THF) was added and the resulting mixture was stirred at room temperature for 3 h. The solvents were evaporated. The residue was purified by ISCO combiflash on silica gel column with MeOH/CH₂Cl₂ (0 to 15% gradient for 30 min) as the eluents to yield 32 mg of the title compound.

¹H NMR (DMSO-d₆) δ 10.09 (s, 1H), 8.3 (s, 1H), 7.93 (s, 1H), 6.76 (bs, 2H), 6.2 (s, 1H), 5.7 (s, 1H), 5.08 (s, 1H), 5.05 (s, 1H), 4.97 (t, 1H, J=4.5), 4.16 (m, 1H), 3.70 (m, 2H), 2.64 (1H, m), 1.14 (d, 3H, J=1.5), 1.11 (d, 3H, J=1.5), 0.81 (s, 3H),

MS (M+1): 418.2

### Example 16

### Preparation of 9-amino-2-(6-hydroxymethyl-3a-methyl-2-oxo-tetrahydro-furo[3,4-d][1,3]dioxol-4-yl)-2,6-dihydro-2,3,5,6-tetraaza-benzo[cd]azulen-7-one (Compound 116)

### Step 1: 9-amino-2-[6-(tert-butyl-diphenyl-silanyloxymethyl)-3a-methyl-2-oxo-tetrahydro-furo[3,4-d][1,3]dioxol-4-yl]-2,6-dihydro-2,3,5,6-tetraaza-benzo[cd]azulen-7-one

To a solution of the product from Example 13, Step 1 (100 mg, 0.17 mmol) in anhydrous DMF (5 mL), CDI (0.069 g, 0.425 mmol) was added under argon. After stirring for 4 h at room temperature, solvents were evaporated. The residue was purified by ISCO combiflash on silica gel column with MeOH/CH₂Cl₂ (0 to 15% gradient for 35 min) as the eluents to yield 103 mg of the target compound.

MS (M+1): 612.2

### Step 2: 9-amino-2-(6-hydroxymethyl-3a-methyl-2-oxo-tetrahydro-furo[3,4-d][1,3]dioxol-4-yl)-2,6-dihydro-2,3,5,6-tetraaza-benzo[cd]azulen-7-one (Compound 116)

To a solution of the product from Step 1 (0.102 g, 0.17 mmol) in anhydrous THF (10 mL), TBAF (340 μL, 0.34 mmol; 1M in THF) was added and the resulting mixture was stirred at room temperature for 30 min. The solvents were evaporated. The residue was purified by ISCO combiflash on silica gel column with MeOH/CH₂Cl₂ (0 to 15% gradient for 30 min) as the eluents to yield 44 mg of the title compound.

¹H NMR (DMSO-d₆) δ 10.17 (s, 1H), 8.34 (s, 1H), 7.96 (s, 1H), 6.7 (bs, 2H), 6.65 (s, 1H), 5.26 (t, 1H, J=4.8), 5.07 (s, 1H), 4.95 (d, 1H, J=3.5), 5.05 (s, 1H), 4.4 (m, 1H), 3.77 (m, 2H), 1.21 (s, 3H).

MS (M+1): 374.0

### Example 17

### Preparation of Acetic Acid 4-acetoxy-2-acetoxymethyl-5-(9-amino-7-oxo-6,7-dihydro-2,3,5,6-tetraaza-benzo[cd]azulen-2-yl)-4-methyl-tetrahydro-furan-3-yl Ester (Compound 117)

To a solution of DCC (1.2 g, 5.76 mmol) in anhydrous DMF (8 mL), AcOH (346 μL, 5.76 mmol) and DMAP (4-dimethylaminopyridine 70 mg, 0.576 mmol) were added under argon. To this mixture, a solution of 9-amino-2-(3,4-dihydroxy-5-hydroxymethyl-3-methyl-tetrahydro-furan-2-yl)-2,6-dihydro-2,3,5,6-tetraaza-benzo[cd]azulen-7-one (compound 100, prepared according to WO 2006/093987, published on Sep. 8, 2006, 0.5 g, 1.44 mmol) in 8 mL of DMF was added. After stirring for 2 h at room temperature, the reaction mixture was quenched with anhydrous MeOH (0.5 mL). The reaction mixture was filtered, and the filtrate was concentrated in vacuo. The residue was purified on ISCO combiflash using 12.0 g silica gel column with MeOH/CH₂Cl₂ (0 to 10% gradient for 30 min) as the eluents to yield 160 mg of the faster moving product, compound 117, The later fractions afforded 245 mg of acetic acid 3-acetoxy-5-(9-amino-7-oxo-6,7-dihydro-2,3,5,6-tetraaza-benzo[cd]azulen-2-yl)-4-hydroxy-4-methyl-tetrahydro-furan-2-ylmethyl ester, compound 135 (see Example 7).

Compound 117:

¹H NMR (DMSO-d₆) δ 10.16 (s, 1H), 8.34 (s, 1H), 7.98 (s, 1H), 6.8 (bs, 2H), 6.57 (s, 1H), 5.42 (d, 1H, J=5.0), 5.07 (s, 1H), 4.43-4.26 (m, 3H), 2.11, 2.06 (2×s, 6H), 1.38 (s, 3H).

MS (M+1): 474.0

### Example 18

### Preparation of Isobutyric Acid 2-(4-acetoxy-5-acetoxymethyl-3-hydroxy-3-methyl-tetrahydro-furan-2-yl)-7-oxo-6,7-dihydro-2H-2,3,5,6-tetraaza-benzo[cd]azulen-9-ylcarbamoyloxymethyl Ester (Compound 118)

To a solution of compound 135 (Example 7, 410 mg, 0.95 mmol) in anhydrous pyridine (9.5 mL), were added pre-activated molecular sieves. The reaction mixture was stirred for 30 min at room temperature, and then Cooled to 0 to 5° C. (ice/water bath). Isobutyryloxymethyl carbonochloridate (Example 9. step 3, 515 μL, 2.85 mmol) was added to the reaction mixture. After stirring for 1.0 h, the reaction mixture was quenched with anhydrous EtOH (0.5 mL). The solvents were evaporated. The residue was purified by ISCO combiflash on silica gel column with MeOH/CH₂Cl₂ (0 to 15% gradient for 35 min) as the eluents to yield 183 mg of the title compound.

¹H NMR (DMSO-d₆) δ 10.82 (s, 1H), 9.91 (s, 1H), 8.37 (s, 1H), 7.91 (s, 1H), 6.6 (s, 1H), 6.22 (s, 1H), 5.87 (s, 1H), 5.79 (s, 2H), 5.82 (d, 1H, J=7.0), 4.35-4.30 (m, 3H), 2.62-2.57 (m, 1H), 2.12, 2.06 (2×s, 6H), 1.15-1.09 (m, 6H), 0.86 (s, 3H).

MS (M+1): 576.2

### Example 19

### Preparation of Isobutyric Acid 4-hydroxy-3-isobutyryloxy-4-methyl-5-[9-(5-methyl-2-oxo-[1,3]dioxol-4-ylmethoxycarbonylamino)-7-oxo-6,7-dihydro-2,3,5,6-tetraaza-benzo[cd]azulen-2-yl]-tetrahydro-furan-2-ylmethyl Ester (Compound 119)

### Step 1: Thiocarbonic Acid S-ethyl Ester O-(5-methyl-2-oxo-[1,3]dioxol-4-ylmethyl) Ester

To an ice cold solution of 4-hydroxymethyl-5-methyl-1,3-dioxol-2-one (see J. Med. Chem., 1999, 42, 3994-4000 for preparation, 3.0 g, 23.07 mmol) in anhydrous ether (120 mL), were added pyridine (1.83 mL, 23.07 mmol) followed by a preformed solution of ethylchlorothiolate (2.7 mL, 25.4 mmol) in ether (25 mL). The reaction mixture was stirred overnight at room temperature, filtered and concentrated in vacuo. The residue was taken up in dichloromethane (200 mL) and washed with sat aq. NaHCO₃, water (3×100 mL). The organic fraction was dried over sodium sulfate. Solvent was evaporated to give the target compound as brown oil (3.2 g).

### Step 2: 4-hydroxymethyl-5-methyl-1,3-dioxol-2-one Carbonochloridate

To a solution of the product from Step 1 (2.0 g, 9.17 mmol) in anhydrous DCM (4.0 mL) cooled to −30° C., was added a preformed solution of SO₂Cl₂ (0.77 mL, 9.17 mmol) in DCM (5 mL). the resulting mixture was stirred for 30 min. The solvents were evaporated to give title compound as light yellow oil (1.5 g).

### Step 3: Isobutyric Acid 4-hydroxy-3-isobutyryloxy-4-methyl-5-[9-(5-methyl-2-oxo-[1,3]dioxol-4-ylmethoxycarbonylamino)-7-oxo-6,7-dihydro-2,3,5,6-tetraaza-benzo[cd]azulen-2-yl]-tetrahydro-furan-2-ylmethyl Ester (Compound 119)

To a solution of compound 135 (Example 7, 150 mg, 0.31 mmol) in anhydrous pyridine (3 mL), were added pre activated molecular sieves. The reaction mixture was stirred for 30 min at room temperature. TMSCl (0.31 mmol) was added and the resulting mixture was stirred for additional 1 h at room temperature. After cooling to −20° C., the product of Step 2,4-hydroxymethyl-5-methyl-1,3-dioxol-2-one carbonochloridate (173 μL, 0.93 mmol) was added to the reaction and stirring was continued for 1 h. The reaction was quenched with anhydrous MeOH (0.5 mL) and the solvents were evaporated. The residue was purified by ISCO combiflash on silica gel column with MeOH/CH₂Cl₂ (0 to 15% gradient for 35 min) as the eluents to yield 109 mg of the title compound.

¹H NMR (DMSO-d₆) δ 10.79 (s, 1H), 9.75 (bs, 1H), 8.37 (s, 1H), 7.92 (s, 1H), 6.69 (s, 1H), 6.25 (s, 1H), 5.88 (s, 1H), 5.21 (d, 1H, J=7.5), 5.09 (s, 2H), 4.36-4.23 (m, 3H), 2.71-2.62 (m, 2H), 1.15-1.02 (m, 6H), 0.87 (s, 3H)

MS (M+1): 644.0

### Example 20

### Preparation of Acetic Acid 3-acetoxy-5-(9-acetoxymethoxycarbonylamino-7-oxo-6,7-dihydro-2,3,5,6-tetraaza-benzo[cd]azulen-2-yl)-4-hydroxy-4-methyl-tetrahydro-furan-2-ylmethyl Ester (Compound 120)

Compound 135 (Example 7, 300 mg, 0.69 mmol) was dissolved in pyridine (7 mL) and chloro-trimethyl silane (882 μL; 1 eq.) was added. The reaction mixture was stirred for 30 minutes. After cooling to 0° C., acyloxymethyl carbonochloridate (Synthesis 1990, 1159-1166, 159 μL, 3 eq.) was added. The reaction was stirred for additional 2 hr at 0° C., then quenched with methanol and the solvents were evaporated. Reverse-phase HPLC (water/acetonitrile) yielded 194 mg (51%) of the final product.

MS: 548.1 (M+H).

¹H-NMR (DMSO-d₆): δ 10.83 (s, 1H), 9.92 (s, 1H), 8.38 (s, 1H), 7.92 (s, 1H), 6.61 (s, 1H), 6.23 (s, 1H), 5.87 (s, 1H), 5.76 (s, 2H), 5.22 (d, 1H), 4.30-4.35 (m, 3H), 2.13 (s, 3H), 2.11 (s, 3H), 2.05 (s, 3H), 0.86 (3H).

### Example 21

### Preparation of 2-amino-3-methyl-butyric acid 2-(3-hydroxy-4-isobutyryloxy-5-isobutyryloxymethyl-3-methyl-tetrahydro-furan-2-yl)-7-oxo-6,7-dihydro-2H-2,3,5,6-tetraaza-benzo[cd]azulen-9-ylcarbamoyloxymethyl Ester (Compound 121)

### Step 1: (S)-2-benzyloxycarbonylamino-3-methyl-butyric acid ethylsulfanylcarbonyl-oxymethyl Ester

Cbz-L-valine (5 g; 19.9 mmol) was converted to its cesium salt by stirring it with cesium carbonate (3.24 g; 0.5 eq) in methanol for 1 hour, followed by evaporation of the solvent and drying overnight over phosphorous pentoxide. This cesium salt was then added to a solution of thiocarbonic acid O-chloromethyl ester S-ethyl ester (3.07 g; 19.9 mmol) in 200 mL DMF and stirred for 2 days at room temperature. The solvents were removed and remaining mixture was mixed with 100 mL of sat. sodium bicarbonate and 100 mL of dichloromethane. The aqueous layer was separated and extracted two more times with dichloromethane. The combined organic fractions were washed with 100 mL of water, dried with sodium sulfate and evaporated. The residue was chromatographed on silica gel using dichloromethane/methanol to give 4.2 g of the title compound.

¹H-NMR (CDCl₃): δ 7.27-7.35 (m, 5H, phenyl), 5.89 (d, 1H, J=5.9 Hz, O—CH—O), 5.77 (d, 1H, J=5.6 Hz, O—CH—O), 5.23 (d, 1H, J=8.8 Hz, NH), 5.10 (s, 2H, Ph-CH₂—O), 4.35 (dd, 1H, J=4.4 Hz, 9.1 Hz, α-CH), 2.88 (q, 2H, J=7.3 Hz, S—CH₂), 2.16-2.22 (m, 1H, β—CH), 1.32 (tr, 3H, J=7.3 Hz, S—CH₂CH₃), 0.98 (d, 3H, J=6.7 Hz, CHCH₃), 0.88 (d, 3H, J=6.8 Hz, CHCH₃).

### Step 2: 2-benzyloxycarbonylamino-3-methyl-butyryloxymenthyl Carbonochloridate

The product of Step 1 (2.0 g; 16 mmol) was dissolved in 15 mL of dry dichloromethane and cooled to −30° C. Sulfuryl chloride (845 μL, 2 eq.) was added dropwise and the reaction was stirred for 30 minutes. Borontrifluorate diethyl etherate (22 μL) was added via syringe and the reaction mixture was allowed to warm to room temperature. After an additional hour of stirring, the solution was evaporated and placed on high vacuum overnight to give the desired product (2.1 g).

¹H-NMR (CDCl₃): δ 7.27-7.30 (m, 5H), 5.84 (d, 1H, J=5.6 Hz), 5.70 (d, 1H, J=5.6 Hz), 5.10-5.15 (m, 1H), 4.30 (dd, 1H, J=4.7 Hz, 8.8 Hz), 2.086-2.17 (m, 1H), 0.93 (d, 3H, J=6.7 Hz), 0.84 (d, 3H, J=7.0 Hz).

### Step 3: 2-benzyloxycarbonylamino-3-methyl-butyric acid 2-(3-hydroxy-4-isobutyryloxy-5-isobutyryloxymethyl-3-methyl-tetrahydro-furan-2-yl)-7-oxo-6,7-dihydro-2H-2,3,5,6-tetraaza-benzo[cd]azulen-9-ylcarbamoyloxymethyl Ester (compound 146)

Compound 134 (Example 5, 35 mg, 0.072 mmol) was dissolved in pyridine (0.5 mL) and chloro-trimethyl silane (8.7 μL; 1 eq.) was added. The reaction mixture was stirred for 30 minutes and then cooled to 0° C. The product of Step 2 (75 μL, 3 eq.) was added. The reaction was stirred for 2 hr at 0° C., then quenched with methanol. The solvents were evaporated. Column chromatography (methanol/dichloromethane), followed by reverse-phase HPLC (water/acetonitrile) yielded 20 mg of the title compound.

¹H-NMR (DMSO-d₆): δ 10.84 (s, 1H), 9.93 (s, 1H), 8.38 (s, 1H), 7.92 (s, 1H), 7.81 (d, 1H, J=7.9 Hz), 7.27-7.32 (m, 5H), 6.59 (s, 1H), 6.24 (s, 1H), 5.90 (s, 1H), 5.87 (d, 1H, J=6.2 Hz), 5.84 (d, 1H, J=6.2 Hz), 5.21 (d, 1H, J=8.8 Hz), 5.02 (s, 2H), 4.24-4.35 (m, 3H), 3.96-4.00 (m, 1H), 2.59-2.69 (m, 2H), 2.05-2.07 (m, 1H), 1.03-1.15 (m, 12H), 0.86-0.90 (m, 9H).

MS: 795.3 (M+H)

### Step 4: 2-amino-3-methyl-butyric acid 2-(3-hydroxy-4-isobutyryloxy-5-isobutyryloxymethyl-3-methyl-tetrahydro-furan-2-yl)-7-oxo-6,7-dihydro-2H-2,3,5,6-tetraaza-benzo[cd]azulen-9-ylcarbamoyloxymethyl Ester (Compound 121)

The product of Step 3 (20 mg, 0.025 mmol) was dissolved in 1 mL of methanol containing 1% acetic acid. Pd/C (10%, 10 mg) was added. The reaction mixture was placed under 1 atm hydrogen atmosphere and stirred vigorously for 1 hour. The palladium catalyst was removed via filtration and the filtrate concentrated in vacuo after addition of 5 mL toluene. The resulting residue was chromatographed using water/acetonitrile containing 0.75% conc. hydrochloric acid to give 2 mg of the title compound.

¹H-NMR (D₂O): δ 7.81 (s, 1H), 7.33 (s, 1H), 6.00 (s, 1H), 5.68-5.75 (m, 3H, C—H), 4.85 (d, 1H), 4.11-4.19 (m, 3H, 4′CH), 3.90 (d, 1H), 2.45-2.49 (m, 2H), 2.17 (m, 1H), 0.91-0.94 (m, 12H), 0.91-0.94 (m, 12H), 0.78-0.83 (m, 6H), 0.67 (s, 3H).

MS: 661.3 (M+H)

### Example 22

### Preparation of 3-morpholin-4-yl-propionic acid 5-(9-amino-7-oxo-6,7-dihydro-2,3,5,6-tetraaza-benzo[cd]azulen-2-yl)-4-hydroxy-4-methyl-3-(3-morpholin-4-yl-propionyloxy)-tetrahydro-furan-2-ylmethyl Ester (Compound 122)

To a solution of 9-amino-2-(3,4-dihydroxy-5-hydroxymethyl-3-methyl-tetrahydro-furan-2-yl)-2,6-dihydro-2,3,5,6-tetraaza-benzo[cd]azulen-7-one (compound 100, prepared according to WO 2006/093987, published on Sep. 8, 2006, 20 mg, 0.058 mmol) in 0.2 mL of DMF, were added 3-morpholin-4-yl-propionic acid hydrochloride (0.23 mmol, 45 mg), pyridine (18 μl, 0.23 mmol), DCC (47.7 mg, 0.23 mmol), and DMAP (0.023 mmol, 2.8 mg). After stirring for 1 h at room temperature, reaction mixture was filtered, and filtrate was concentrated in vacuo upto dryness. Residue was purified on ISCO combiflash using 12.0 g silica gel column with MeOH/CH₂Cl₂ (0 to 45% gradient for 30 min) as the eluents to yield 10.2 mg of the title compound.

¹H NMR (DMSO-d₆): δ 10.11 (s, 1H), 8.31 (s, 1H), 7.9 (s, 1H), 6.81 (bs, 2H), 6.23 (s, 1H), 5.85 (s, 1H), 5.19 (d, 1H, J=8.1), 5.06 (s, 1H, J=1.8), 4.37-4.33 (m, 3H), 3.53-3.48 (m, 8H), 3.32-2.36 (m, 8H), 2.33-2.3 (m, 8H), 0.83 (s, 3H).

MS (M+1): 630.2

### Example 23

### Preparation of Isobutyric Acid 2-(3,4-diacetoxy-5-acetoxymethyl-3-methyl-tetrahydro-furan-2-yl)-7-oxo-6,7-dihydro-2H-2,3,5,6-tetraaza-benzo[cd]azulen-9-ylcarbamoyloxymethyl Ester (Compound 123)

Into a solution of compound 117 (Example 17, 400 mg, 0.85 mmol) in anhydrous pyridine (8 mL) was added TMSCl (107 μL, 0.85 mmol) and the resulting mixture stirred at room temperature for 0.5 h. The mixture was then cooled to 0° C. and isobutyryloxymethyl chloroformate (0.46 g, 2.6 mmol) was added. After 1 h stirring at 0° C. the reaction was quenched with MeOH and concentrated. The residue was purified by column chromatography on silica gel using 0-7% gradient of MeOH in CH₂Cl₂ to yield the target compound as a pale yellow solid after crystallization from MeOH (270 mg, 51%).

¹H NMR (DMSO-d₆): δ 10.82 (s, 1H), 9.84 (s, 1H), 8.40 (s, 1H), 8.06 (s, 1H), 6.66 (d, 1H, J=1.8 Hz), 6.61 (s, 1H), 5.79 (s, 2H), 5.43 (d, 1H, J=6.2 Hz), 4.42-4.24 (m, 3H), 2.62 (heptet, 1H, J=7.0 Hz), 2.12 (s, 3H), 2.08 (s, 3H), 2.05 (s, 3H), 1.38 (s, 3H), 1.11 (d, 6H, J=7.0 Hz).

MS: m/z=618.7 (M+1).

### Example 24

### Preparation of 2-Acetylamino-3-methyl-butyric acid 5-(9-amino-7-oxo-6,7-dihydro-2,3,5,6-tetraaza-benzo[cd]azulen-2-yl)-3,4-dihydroxy-4-methyl-tetrahydro-furan-2-ylmethyl Ester (Compound 124)

Preparation of 1.2 eq. of activated N-acetyl-L-valine mixture: N-acetyl-L-valine (82.5 mg, 1.2 eq.) and HATU (197 mg, 1.2 eq.) were dissolved in 4 mL of dry DMF. Diisopropylethylamine (90.2 μL, 1.2 eq) was added and the mixture stirred for 10 minutes.

On day 1, 1.2 eq. of activated N-acetyl-L-valine mixture was prepared and added to solid compound 100 (150 mg, 0.431 mmol) and the reaction mixture was stirred overnight. On day 2, another 1.2 eq. of activated N-acetyl-L-valine mixture was prepared and added to the reaction mixture and stirring continued overnight. On day 3, additional 1.2 eq. of activated N-acetyl-L-valine mixture was prepared and added to the reaction mixture. Again, the reaction mixture was stirred overnight.

On day 4, the solvents were removed and the residue was purified by column chromatography (methanol/dichloromethane). Fractions containing the product were re-chromatographed using reverse phase HPLC to give 25 mg of compound 124.

¹H-NMR (DMSO-d₆): δ 10.02 (s, 1H), 8.25 (s, 1H), 8.09 (m, 1H), 7.73 (s, 1H), 6.72 (br s, 2H), 6.14 (s, 1H), 5.43 (d, 1H), 5.35 (s, 1H), 4.97 (s, 1H), 4.34-4.43 (m, 2H), 4.01-4.18 (m, 2H), 3.70-3.82 (m, 1H), 1.91-1.97 (m, 1H), 1.82 (s, 3H), 0.67-0.87 (m, 9H).

MS: 489.2 (M+H)

### Example 25

### Preparation of Isobutyric Acid 4-hydroxy-2-hydroxymethyl-5-(9-isobutyryloxymethoxycarbonylamino-7-oxo-6,7-dihydro-2,3,5,6-tetraaza-benzo[cd]azulen-2-yl)-4-methyl-tetrahydro-furan-3-yl Ester (Compound 125)

### Step 1: 9-amino-2-[5-(di-tert-butyl-hydroxy-silanyloxymethyl)-3,4-dihydroxy-3-methyl-tetrahydro-furan-2-yl]-2,6-dihydro-2,3,5,6-tetraaza-benzo[cd]azulen-7-one

To a solution of 9-amino-2-(3,4-dihydroxy-5-hydroxymethyl-3-methyl-tetrahydro-furan-2-yl)-2,6-dihydro-2,3,5,6-tetraaza-benzo[cd]azulen-7-one (compound 100, prepared according to WO 2006/093987, published on Sep. 8, 2006, 2.5 g, 7.2 mmol) in DMF (28,86 mL) was added imidazole (2.94 g, 43.2 mmol) and followed by the dropwise addition of di-tert-butylsilyl bis(trifluoromethane sulfonate) (2.7 mL, 7.28 mmol) under rapid stirring. The reaction mixture was stirred at room temperature for 3 hours and then quenched with MeOH, concentrated in vacuo onto celite and purified on Isco CombiFlash purification system utilizing a 40 g silica gel column and 0-20% MeOH gradient in DCM as the eluent over 20 minutes to afford 2.25 g (64%) of 9-amino-2-(2,2-di-tert-butyl-7-hydroxy-7-methyl-tetrahydro-furo[3,2-d][1,3,2]dioxasilin-yl)-2,6-dihydro-2,3,5,6-tetraaza-benzo[cd]azulen-7-one (see also example 4, step 1) along with 80 mg of target compound.

MS: m/z=506.2 (M+1)

### Step 2: Isobutyric Acid 5-(9-amino-7-oxo-6,7-dihydro-2,3,5,6-tetraaza-benzo[cd]azulen-2-1)-2-(di-tert-butyl-hydroxy-silanyloxymethyl)-4-hydroxy-4-methyl-tetrahydro-furan-3-yl Ester

Compound from Step 1 (80 mg, 0.16 mmol) was added into a mixture of DCC (130.4 mg, 0.63 mmol), DMAP (7.7 mg, 0.063 mmol) and isobutyric acid (58.7 μL, 0.632 mmol) in anhydrous DMF (1.58 mL). After an overnight stirring at room temperature the reaction was quenched with MeOH and white solid filtered off. The evaporated residue was triturated with MeOH, filtered and evaporated. Silica gel column chromatography with CH₂Cl₂/MeOH (gradient 0-10% MeOH) yielded the target compound as pale-yellow solid (50 mg, 55%).

MS: m/z=576.2 (M+1)

### Step 3: Isobutyric Acid 2-(di-tert-butyl-hydroxy-silanyloxymethyl)-4-hydroxy-5-(9-sobutyryloxymethoxycarbonylamino-7-oxo-6,7-dihydro-2,3,5,6-tetraaza-benzo[cd]azulen-2-1)-4-methyl-tetrahydro-furan-3-yl Ester

To an ice-cold solution of the product from Step 2 (50 mg, 0.087 mmol) and DMAP (2.12 mg, 0.0174 mmol) in anhydrous pyridine (0.87 mL) was added isobutyryoxymethyl chloroformate (47 mg, 0.261 mmol). Reaction mixture was stirred at 0° C. for 50 min then quenched with MeOH and evaporated. Purification on a silica gel column with CH₂Cl₂/MeOH (gradient 0-10% MeOH) yielded the target compound as pale-yellow foam (32 mg, 51%).

MS: m/z=720.3 (M+1).

### Step 4: Isobutyric Acid 4-hydroxy-2-hydroxymethyl-5-(9-isobutyryloxymethoxy-carbonylamino-7-oxo-6,7-dihydro-2,3,5,6-tetraaza-benzo[cd]azulen-2-yl)-4-methyl-tetrahydro-furan-3-yl Ester (Compound 125)

To a solution of compound from Step 3 (32 mg, 0.044 mmol) in THF (0.5 mL) was added Et₃N.3HF (65 μL, 0.4 mmol) and the resulting mixture was stirred for 6 days at room temperature. The reaction was quenched with silica and evaporated to dryness. Purification by HPLC yielded 15 mg (60%) of the target compound.

¹H NMR (DMSO-d₆, 300 MHz): δ 10.793 (s, 1H), δ 9.871 (s, 1H), 8.364 (s, 1H), 8.025 (s, 1H), 6.576 (s, 1H), 6.203 (s, 1H), 5.796 (s, 2H), 5.710 (s, 1H), 5.103 (d, 1H, J=8.7 Hz), 5.02 (t, 1H, J=5.4 Hz), 4.180-4.120 (m, 1H), 3.72-3.66 (m, 2H), 2.67-2.580 (heptet, 2H, J=7.1 Hz), 1.119-1.077 (m, 12H), 0.821 (s, 3H);

MS (M+1): 562.2.

### Example 26

### Preparation of [2-(3,4-Dihydroxy-5-hydroxymethyl-3-methyl-tetrahydro-furan-2-yl)-7-oxo-6,7-dihydro-2H-2,3,5,6-tetraaza-benzo[cd]azulen-9-yl]-carbamic acid 5-methyl-2-oxo-[1,3]dioxol-4-ylmethyl Ester (Compound 126)

### Step 1: [2-(2,2-Di-tert-butyl-7-hydroxy-7-methyl-tetrahydro-furo[3,2-d][1,3,2]dioxasilin-6-yl)-7-oxo-6,7-dihydro-2H-2,3,5,6-tetraaza-benzo[cd]azulen-9-yl]-carbamic acid 5-methyl-2-oxo-[1,3]dioxol-4-ylmethyl Ester

To a solution of 9-amino-2-(2,2-di-tert-butyl-7-hydroxy-7-methyl-tetrahydro-furo[3,2-d][1,3,2]dioxasilin-6-yl)-2,6-dihydro-2,3,5,6-tetraaza-benzo[cd]azulen-7-one (Example 4, step 1, 120 mg, 0.25 mmol) in anhydrous pyridine (2.5 mL), were added pre-activated molecular sieves. The reaction mixture was stirred for 30 min at room temperature. TMSCl (0.25 mmol) was added and the resulting mixture was stirred for additional 1 h at room temperature and then cooled to 0 to 5° C. (ice/water bath). 4-Hydroxymethyl-5-methyl-1,3-dioxol-2-one carbonochloridate (Example 19, Step 2, 236 μL, 1.23 mmol) was added to the reaction mixture. After stirring for 1.0 h, the reaction was quenched with anhydrous MeOH (0.5 mL). The solvents were evaporated. The residue was purified by ISCO combiflash on silica gel column with MeOH/CH₂Cl₂ (0 to 15% gradient for 35 min) as the eluents to yield 39 mg of the target compound.

MS (M+1): 644.2

### Step 2: [2-(3,4-dihydroxy-5-hydroxymethyl-3-methyl-tetrahydro-furan-2-yl)-7-oxo-6,7-dihydro-2H-2,3,5,6-tetraaza-benzo[cd]azulen-9-yl]-carbamic acid 5-methyl-2-oxo-[1,3]dioxol-4-ylmethyl Ester (Compound 126)

To the product from step 1 (0.039 g, 0.06 mmol) dissolved in anhydrous THF (2 mL), was added Et₃N.3HF (12 μL, 0.07 mmol) at 0 to 5° C. The resulting mixture stirred for 30 min. The mixture is concentrated in vacuo and the crude material is taken up in DMF: H₂O (8:2), and purified by Phenomenex-C₁₈ reverse phase HPLC using a 0-99% B gradient over 30 min at 10 mL/min (Buffer A=H₂O, Buffer B=acetonitrile) to afford 11.6 mg of the title compound.

¹H NMR (DMSO-d₆) δ 10.73 (s, 1H), 9.69 (bs, 1H), 8.34 (s, 1H), 7.95 (s, 1H), 6.62 (s, 1H), 6.17 (s, 1H), 5.27 (s, 1H), 5.15 (bs, 1H), 5.07 (s, 2H), 4.89 (t, 1H, J=4.5), 3.90-3.76 (m, 4H), 2.20 (s, 3H), 0.77 (s, 3H).

MS (M+1): 504.1

### Example 27

### Preparation of Propionic Acid 5-(9-amino-7-oxo-6,7-dihydro-2,3,5,6-tetraaza-benzo[cd]azulen-2-yl)-4-methyl-3,4-bis-propionyloxy-tetrahydro-furan-2-ylmethyl Ester (Compound 127)

To a solution of 9-amino-2-(3,4-dihydroxy-5-hydroxymethyl-3-methyl-tetrahydro-furan-2-yl)-2,6-dihydro-2,3,5,6-tetraaza-benzo[cd]azulen-7-one (compound 100, prepared according to WO 2006/093987, published on Sep. 8, 2006, 25 mg, 0.072 mmol) in DMF (0.36 mL) was added DCC (89 mg, 0.43 mmol), DMAP (8.8 mg, 0.072 mmol) and propionic acid (32.3 μL, 0.43 mmol). The reaction was stirred at room temperature overnight. The crude product was concentrated and purified by HPLC to give 20 mg of title compound.

¹H NMR (DMSO-d₆, 300 MHz): δ 10.145 (s, 1H), 8.345 (s, 1H), 7.988 (s, 1H), 6.801 (s, 2H), 6.568 (s, 1H), 5.452 (d, 1H, J=5.7 Hz), 5.072 (d, 1H, J=1.5 Hz), 4.441-4.250 (m, 3H), 2.430-2.310 (m, 6H), 1.369 (s, 3H), 1.086-0.977 (m, 9H);

MS (M+1): 516.2.

### Example 28

### Preparation of Isobutyric Acid 4-hydroxy-3-isobutyryloxy-5-(9-isobutyryloxy-methoxycarbonylamino-7-oxo-6,7-dihydro-2,3,5,6-tetraaza-benzo[cd]azulen-2-yl)-4-methyl-tetrahydro-furan-2-ylmethyl Ester (Compound 128)

To a solution of compound 134 (Example 5, 400 mg, 0.82 mmol) in pyridine (4.1 mL) were added dimethylaminopyridine (20 mg, 0.164 mmol) and molecular sieves. The mixture was stirred at room temperature for 1 hour then isobutyryl-oxymethyl chloroformate was added (440 μL, 2.46 mmol). The reaction was stirred at room temperature overnight. The reaction was quenched by addition of methanol and the mixture was concentrated in vacuuo. The product was purified by HPLC to give 312 mg of the title compound.

¹H NMR (DMSO-d₆, 300 MHz): δ 10.815 (d, 1H, J=1.5 Hz), 9.886 (s, 1H), 8.380 (s, 1H), 7.918 (s, 1H), 6.626 (d, 1H, J=1.8 Hz), 6.250 (s, 1H), 5.888 (s, 1H), 5.813 (s, 2H), 5.237-5.209 (d, 1H, J=8.4 Hz), 4.354-4.200 (m, 3H), 2.70-2.55 (m, 3H), 1.158-1.032 (m, 18H), 0.874 (s, 3H);

MS (M+1): 632.2.

### Example 29

### Preparation of Isobutyric Acid 3,4-dihydroxy-5-(9-isobutyryloxymethoxy-carbonylamino-7-oxo-6,7-dihydro-2,3,5,6-tetraaza-benzo[cd]azulen-2-yl)-4-methyl-tetrahydro-furan-2-ylmethyl Ester (Compound 129)

Following the procedure for the preparation of compound 128, the title compound was isolated as an additional product through HPLC purification.

¹H NMR (DMSO-d₆, 300 MHz): δ 10.783 (d, 1H, J=1.8 Hz), 9.820 (s, 1H), 8.365 (s, 1H), 7.856 (s, 1H), 6.628 (d, 1H, J=1.8 Hz), 6.205 (s, 1H), 5.806 (s, 1H), 5.813 (s, 2H), 5.448-5.412 (m, 2H,), 4.466-4.420 (m, 1H), 4.265-4.207 (m, 1H), 4.129-4.000 (m, 2H), 2.680-2.56 (m, 2H), 1.121-1.018 (m, 12H), 0.813 (s, 3H);

MS (M+1): 562.2.

### Example 30

### Preparation of Propionic Acid 5-(9-amino-7-oxo-6,7-dihydro-2,3,5,6-tetraaza-benzo[cd]azulen-2-yl)-4-hydroxy-4-methyl-3-propionyloxy-tetrahydro-furan-2-ylmethyl Ester (Compound 130)

To a solution of 9-amino-2-(3,4-dihydroxy-5-hydroxymethyl-3-methyl-tetrahydro-furan-2-yl)-2,6-dihydro-2,3,5,6-tetraaza-benzo[cd]azulen-7-one (compound 100, prepared according to WO 2006/093987, published on Sep. 8, 2006, 25 mg, 0.072 mmol) in DMF (0.7 mL) was added DCC (59.3 mg, 0.29 mmol), DMAP (3.5 mg, 0.029 mmol) and propionic acid (21.5 μL, 0.29 mmol). The reaction was stirred at room temperature for one hour. The crude product was concentrated and purified by HPLC to give 15 mg of title compound.

¹H NMR (DMSO-d₆, 300 MHz): δ 10.138 (s, 1H), 8.318 (s, 1H), 7.877 (s, 1H), 6.826 (s, 2H), 6.219 (s, 1H), 5.848 (s, 1H), 5.178-5.151 (d, 1H, J=8.1 Hz), 5.055 (s, 1H), 4.354 (m, 3H), 2.450-2.300 (m, 4H), 1.086-0.985 (m, 6H), 0.812 (s, 3H);

MS (M+1): 460.2.

### Example 31

### Preparation of Propionic Acid 5-(9-amino-7-oxo-6,7-dihydro-2,3,5,6-tetraaza-benzo[cd]azulen-2-yl)-3,4-dihydroxy-4-methyl-tetrahydro-furan-2-ylmethyl Ester (Compound 131)

Following the procedure for the preparation of compound 130, the title compound was isolated as an additional product through HPLC purification.

¹H NMR (DMSO-d₆, 300 MHz): δ 10.095-10.089 (d, 1H, J=1.8 Hz), 8.307 (s, 1H), 7.769 (s, 1H), 6.767 (s, 2H), 6.197 (s, 1H), 5.500-5.477 (d, 1H, J=6.9 Hz), 5.412 (s, 1H), 5.037-5.031 (d, 1H, J=1.8 Hz), 4.453-4.420 (m, 1H), 4.359-4.293 (m, 1H), 4.140-4.100 (m, 1H), 3.892-3.837 (m, 1H), 2.392-2.320 (m, 2H), 1.013 (t, 3H, J=7.8 Hz); 0.768 (s, 3H);

MS (M+1): 404.2.

### Example 32

### Preparation of Isobutyric Acid 5-(9-amino-7-oxo-6,7-dihydro-2,3,5,6-tetraaza-benzo[cd]azulen-2-yl)-3,4-bis-isobutyryloxy-4-methyl-tetrahydro-furan-2-ylmethyl Ester (Compound 132)

To a solution of 9-amino-2-(3,4-dihydroxy-5-hydroxymethyl-3-methyl-tetrahydro-furan-2-yl)-2,6-dihydro-2,3,5,6-tetraaza-benzo[cd]azulen-7-one (compound 100, prepared according to WO 2006/093987, published on Sep. 8, 2006, 25 mg, 0.072 mmol) in DMF (0.36 mL) was added DCC (89 mg, 0.43 mmol), DMAP (8.8 mg, 0.072 mmol) and isobutyric acid (40 μL, 0.43 mmol). The reaction was stirred at room temperature overnight. The crude product was concentrated and purified by HPLC to give 17 mg of title compound.

¹H NMR (DMSO-d₆, 300 MHz): δ 10.155 (s, 1H), 8.347 (s, 1H), 7.993 (s, 1H), 6.800 (s, 2H), 6.579 (s, 1H), 5.489 (d, 1H, J=5.4 Hz), 5.070 (d, 1H, J=1.8 Hz), 4.400-4.300 (m, 3H), 2.650-2.510 (m, 3H), 1.328 (s, 3H), 1.148-1.062 (m, 18H);

MS (M+1): 558.2.

### Example 33

### Preparation of Isobutyric Acid 4-acetoxy-5-(9-amino-7-oxo-6,7-dihydro-2,3,5,6-tetraaza-benzo[cd]azulen-2-yl)-3-isobutyryloxy-4-methyl-tetrahydro-furan-2-ylmethyl Ester (Compound 133)

To a solution of compound 134 (Example 5, 25 mg, 0.05 mmol) in DMF (0.26 mL) was added DCC (42.3 mg, 0.205 mmol), DMAP (2.5 mg, 0.0205 mmol) and acetic acid (12 μL, 0.205 mmol). The reaction was stirred at room temperature for 3 days. The crude product was concentrated and purified by HPLC to give 11 mg of title compound.

¹H NMR (DMSO-d₆, 300 MHz): δ 10.170 (s, 1H), 8.346 (s, 1H), 7.997 (s, 1H), 6.803 (s, 2H), 6.559 (s, 1H), 5.440 (d, 1H, J=5.4 Hz), 5.070 (d, 1H, J=1.8 Hz), 4.420-4.240 (m, 3H), 2.620-2.500 (m, 2H), 2.093 (s, 3H), 1.350 (s, 3H), 1.148-1.061 (m, 12H);

MS (M+1): 530.2.

### Example 34

### Preparation of Isobutyric Acid 4-acetoxy-5-acetoxymethyl-2-(9-amino-7-oxo-6,7-dihydro-2,3,5,6-tetraaza-benzo[cd]azulen-2-yl)-3-methyl-tetrahydro-furan-3-yl Ester (Compound 136)

To a solution of compound 135 (Example 7, 27 mg, 0.063 mmol) in DMF (0.31 mL) was added DCC (51.6 mg, 0.25 mmol), DMAP (3.05 mg, 0.025 mmol) and isobutyric acid (23 μL, 0.25 mmol). The reaction was stirred at room temperature for 36 hours. The crude product was concentrated and purified by HPLC to give 11 mg of title compound.

¹H NMR (DMSO-d₆, 300 MHz): δ 10.131 (s, 1H), 8.343 (s, 1H), 7.981 (s, 1H), 6.790 (s, 2H), 6.577 (s, 1H), 5.40 (d, 1H, J=5.4 Hz), 5.063 (d, 1H, J=1.5 Hz), 4.420-4.230 (m, 3H), 2.720-2.60 (m, 1H), 2.056 (s, 3H), 2.038 (s, 3H), 1.373 (s, 3H), 1.138-1.11 (m, 6H);

MS (M+1): 502.2.

### Example 35

### Preparation of Acetic Acid 4-acetoxy-2-acetoxymethyl-5-(9-amino-7-oxo-6,7-dihydro-2,3,5,6-tetraaza-benzo[cd]azulen-2-yl)-4-methyl-tetrahydro-furan-3-yl Ester (Compound 117)

The starting material compound 100 (100 mg) was co-evaporated three times with anhydrous pyridine, and left on high vacuum for overnight before reaction. To a solution of 9-amino-2-(3,4-dihydroxy-5-hydroxymethyl-3-methyl-tetrahydro-furan-2-yl)-2,6-dihydro-2,3,5,6-tetraaza-benzo[cd]azulen-7-one (compound 100, 100 mg, 0.29 mmol) and DCC (357 mg, 1.73 mmol, 6 equivalent) in anhydrous DMF (source Aldrich 99.8%) (2.9 mL. 0.1 M solution of compound 100 in DMF), was added DMAP (211 mg, 1.73 mmol, 6 equivalent), followed by AcOH (source: Aldrich, Reagent plus, >99%) (104 μL, 1.73 mmol, 6 equivalent) under argon. Reaction was monitored by HPLC after 2 h, 6 h, and 22 h. After stirring for 22 h at room temperature, the reaction mixture was filtered, and reaction flask was washed with DMF (2×3 ml), and washings were filtered. Filtrates were combined and added 0.5 ml of MeOH, and stirred for 5 min at room temperature. Resulting solution was concentrated in vacuo till no residual solvents. The residue left was re-dissolved in 10% MeOH in DCM (10 ml), and was adsorbed on celite. Solvents were evaporated in-vacuo, and was purified on ISCO combiflash using 40.0 g silica gel column with MeOH/CH₂Cl₂ (0 to 10% gradient for 30 min) as the eluents to yield 75.0 mg (55% isolated yield) of the desired compound 117.

### Example 36

### Preparation of [2-(3,4-Dihydroxy-5-hydroxymethyl-3-methyl-tetrahydro-furan-2-yl)-7-oxo-6,7-dihydro-2H-2,3,5,6-tetraaza-benzo[cd]azulen-9-yl]-carbamic Acid Pentyl Ester (Compound 138)

### Step 1: 9-Amino-2-(2,2-di-tert-butyl-7-hydroxy-7-methyl-tetrahydro-furo[3,2-d][1,3,2]dioxasilin-6-yl)-2,6-dihydro-2,3,5,6-tetraaza-benzo[cd]azulen-7-one

To a solution of compound 100 (500 mg, 1.441 mmol) in DMF (5.76 mL) was added imidazole followed by the dropwise addition of di-tert-butylsilyl bis(trifluoromethane sulfonate) under rapid stirring. The reaction mixture was stirred at room temperature for 3 hours then quenched with MeOH, concentrated in vacuo onto celite and purified on Isco CombiFlash purification system utilizing a 40 g silica gel column and 0-20% MeOH gradient in DCM as the eluent over 20 minutes to afford 450 mg (64%).

MS: m/z=488.2 (M+1)

### Step 2: [2-(2,2-Di-tert-butyl-7-hydroxy-7-methyl-tetrahydro-furo[3,2-d][1,3,2]dioxasilin-6-yl)-7-oxo-6,7-dihydro-2H-2,3,5,6-tetraaza-benzo[cd]azulen-9-yl]-carbamic Acid Pentyl Ester

To a solution of the product from step 1 (200 mg, 0.411 mmol) in pyridine (1.65 mL) was added DMAP (63 mg, 0.513 mmol) and chloroformic acid n-amylester (178 μL, 1.232 mmol) and the reaction was stirred at room temperature overnight. The reaction was quenched with methanol, concentrated in vacuo onto celite and purified on Isco CombiFlash purification system utilizing a 12 g silica gel column and 0-10% MeOH gradient in DCM as the eluent over 20 minutes to afford 135 mg (55%).

MS: m/z=602.3 (M+1)

### Step 3: [2-(3,4-Dihydroxy-5-hydroxymethyl-3-methyl-tetrahydro-furan-2-yl)-7-oxo-6,7-dihydro-2H-2,3,5,6-tetraaza-benzo[cd]azulen-9-yl]-carbamic Acid Pentyl Ester (Compound 138)

To a solution of the product from step 2 (132 mg, 0.220 mmol) in THF (1.1 mL) was added TBAF (549 μl, 1Molar solution in THF) at 0° C. and the reaction was allowed to warm to room temperature and stir for 15 minutes. The reaction was quenched with the addition of silica gel, concentrated in vacuo and purified on Isco CombiFlash purification system utilizing a 4 g silica gel column and 0-20% MeOH gradient in DCM as the eluent over 20 minutes followed by a second purification on reverse phase HPLC (0-100% buffer B over 30 minutes at 10 mL/min flow rate —Buffer A=H₂O; Buffer B=ACN) to afford 45 mg (44%) of compound 138.

¹H NMR (DMSO-d₆): δ 10.67 (d, 1H, J=1.5 Hz), 9.46 (s, 1H), 8.35 (s, 1H), 7.97 (s, 1H), 6.62 (d, 1H, J=1.5 Hz), 6.19 (s, 1H), 5.26 (s, 1H) 5.15 (d, 1H, J=6.6 Hz), 4.89 (t, 1H, J=5.4 Hz), 4.14 (t, 2H, J=6.6 Hz), 3.99-3.65 (m, 4H), 1.67 (m, 2H), 1.35 (m, 4H), 0.90 (t, 3H, J=6.6 Hz), 0.79 (s, 3H).

MS: m/z=462.2 (M+1)

### Example 37

### Preparation of Isobutyric Acid 5-(9-amino-7-oxo-6,7-dihydro-2,3,5,6-tetraaza-benzo[cd]azulen-2-yl)-2-hydroxymethyl-4-isobutyryloxy-4-methyl-tetrahydro-furan-3-yl Ester (Compound 139)

### Step 1: 2-(5-O-tert-Butyldimethoxysilyl-2,3-bis-O-isobutyryl-2-C-methyl-β-D-ribofuanosyl)-2,6-dihydro-7H-2,3,5,6-tetraazabenzo[cd]azulen-7-one

Compound from Example 10, Step 1 (400 mg, 0.87 mmol) was added into a pre-stirred mixture of DCC (448 mg, 2.2 mmol), DMAP (42 mg, 0.35 mmol) and isobutyric acid (202 μL, 2.2 mmol) in DMF (5 mL) over molecular sieves (4 Å). The resulting reaction mixture was stirred overnight at room temperature. Another portion of DCC (448 mg, 2.2 mmol), DMAP (42 mg, 0.35 mmol) and isobutyric acid (202 μL, 2.2 mmol) was added then and stirring continued for 1 day. At this point the reaction mixture was diluted with MeOH, solid material filtered and filtrate evaporated. The residue was purified by column chromatography on silica gel using 0-10% gradient of MeOH in CH₂Cl₂ to yield 300 mg of the target compound (57%).

MS: m/z=602.3 (M+1).

### Step 2: Isobutyric Acid 5-(9-amino-7-oxo-6,7-dihydro-2,3,5,6-tetraaza-benzo[cd]azulen-2-yl)-2-hydroxymethyl-4-isobutyryloxy-4-methyl-tetrahydro-furan-3-yl Ester (Compound 139)

To a solution of compound from Step 1 (232 mg, 0.39 mmol) in THF (4 mL) were added Et₃N (108 μL, 0.78 mmol) and Et₃N.3HF (63 μL, 0.39 mmol). The resulting mixture was stirred at room temperature for 4 h and then evaporated. The residue was purified by column chromatography on silica gel using 0-10% gradient of MeOH in CH₂Cl₂ to yield the target compound as a white solid (120 mg, 63%).

¹H NMR (DMSO-d₆): δ 10.10 (s, 1H), 8.34 (s, 1H), 8.03 (s, 1H), 6.77 (br s, 2H), 6.53 (s, 1H), 5.37 (d, 1H, J=3.4 Hz), 5.11 (m, 1H), 5.07 (d, 1H, J=0.8 Hz), 4.09 (dd, 1H, J=6.0 Hz and 2.6 Hz), 3.72 (m, 2H), 2.56 (m, 2H), 1.34 (s, 3H), 1.13, 1.12 (2d, 2×3H, J=4.6 Hz), 1.08, 1.05 (2d, 2×3H, J=48 Hz).

MS: m/z=488.2 (M+1).

### Example 38

### Preparation of Isobutyric Acid 5-(9-benzyloxycarbonylamino-7-oxo-6,7-dihydro-2,3,5,6-tetraaza-benzo[cd]azulen-2-yl)-4-hydroxy-3-isobutyryloxy-4-methyl-tetrahydro-furan-2-ylmethyl Ester (Compound 141)

To a solution of compound 134 (35 mg, 0.071 mmol) in pyridine (3 mL) was added benzyl chloroformate (51.3 μL, 0.359 mmol). The reaction was stirred at room temperature for overnight. The crude product was concentrated and purified by HPLC to give 12 mg of title compound.

¹H NMR (DMSO-d₆, 300 MHz): δ 10.748 (s, 1H), 9.659 (s, 1H), 8.367 (s, 1H), 7.918 (s, 1H), 7.475-7.348 (m, 5H), 6.701 (s, 1H), 6.240 (s, 1H), 5.857 (s, 1H), 5.210 (m, 3H), 4.330 (m, 3H), 2.70-2.50 (m, 2H), 1.142-0.99 (m, 12H), 0.869 (s, 3H);

MS (M+1): 622.2.

### Example 39

### Preparation of 3-Morpholin-4-yl-propionic acid 4-acetoxy-2-acetoxymethyl-5-(9-amino-7-oxo-6,7-dihydro-2,3,5,6-tetraaza-benzo[cd]azulen-2-yl)-4-methyl-tetrahydro-furan-3-yl Ester (Compound 142)

### Step 1: 9-amino-2-[5-(tert-butyl-dimethyl-silanyloxymethyl)-3,4-dihydroxy-3-methyl-tetrahydro-furan-2-yl]-2,6-dihydro-2,3,5,6-tetraaza-benzo[cd]azulen-7-one

To a solution of 9-amino-2-(3,4-dihydroxy-5-hydroxymethyl-3-methyl-tetrahydro-furan-2-yl)-2,6-dihydro-2,3,5,6-tetraaza-benzo[cd]azulen-7-one (Compound 100, prepared according to WO 2006/093987, published on Sep. 8, 2006, 550 mg, 1.59 mmol) in DMF (16 mL) was added imidazole (323 mg, 4.76 mmol) followed by the dropwise addition of tert-butyldimethylsilyl chloride in DMF (3 mL) under rapid stirring. The reaction was stirred at room temperature and monitored by QC-HPLC. After 1 hour, the reaction was quenched with MeOH, concentrated in vacuo onto celite and purified on Isco CombiFlash purification system utilizing a 40 g silica gel column and 0-30% MeOH gradient in DCM as the eluent over 20 minutes to afford 300 mg (41%) of the desired product.

MS: m/z=462.2 (M+1)

### Step 2: 3-Morpholin-4-yl-propionic acid 5-(9-amino-7-oxo-6,7-dihydro-2,3,5,6-tetraaza-benzo[cd]azulen-2-yl)-2-(tert-butyl-dimethyl-silanyloxymethyl)-4-hydroxy-4-methyl-tetrahydro-furan-3-yl Ester

To a solution of DCC (44.6 mg, 0.217 mmol), DMAP (5.27 mg, 0.0.043 mmol), pyridine (36 μL, 0.432 mmol), and 3-morpholin-4-yl-propionic acid hydrochloride (42.12 mg, 0.22 mmol) in DMF (0.15 mL) was added (9-amino-2-[5-(tert-butyl-dimethyl-silanyloxymethyl)-3,4-dihydroxy-3-methyl-tetrahydro-furan-2-yl]-2,6-dihydro-2,3,5,6-tetraaza-benzo[cd]azulen-7-one (50 mg, 0.11 mmol). The reaction was stirred at room temperature overnight. The crude product was concentrated and purified by HPLC to give 20 mg of the desired product.

### Step 3: 3-Morpholin-4-yl-propionic acid 5-(9-amino-7-oxo-6,7-dihydro-2,3,5,6-tetraaza-benzo[cd]azulen-2-yl)-4-hydroxy-2-hydroxymethyl-4-methyl-tetrahydro-furan-3-yl Ester

To a solution of the product from Step 2 (50 mg, 0.083 mmol) in THF (0.6 mL) was added TEA.3HF (14 μL, 0.0596 mmol) at 0° C. The mixture was allowed to warm to room temperature and monitored by QC-HPLC. After 1 hour a second 10 μL of TEA.3HF was added and continued monitoring via QC-HPCL. Reaction was complete after 2.5 hours. The crude mixture was purified by HPLC (0 to 40% MeOH in CH₂Cl₂) afford 30 mg of the desired product.

### Step 4: 3-Morpholin-4-yl-propionic acid 4-acetoxy-2-acetoxymethyl-5-(9-amino-7-oxo-6,7-dihydro-2,3,5,6-tetraaza-benzo[cd]azulen-2-yl)-4-methyl-tetrahydro-furan-3-yl Ester (Compound 142)

To a solution of the product from Step 3 (17 mg, 0.035 mmol) in DMF (0.2 mL) was added DCC (28.7 mg, 0.139 mmol), DMAP (4.25 mg, 0.035 mmol) and acetic acid (8.85 μL, 0.15 mmol). The reaction was stirred at room temperature for 1 hour and heated to 50° C. for one hour. The crude product was concentrated and purified by HPLC to give 17 mg of title compound.

¹H NMR (DMSO-d₆, 300 MHz): δ 10.157 (s, 1H), 8.343 (s, 1H), 7.989 (s, 1H), 6.812 (s, 2H), 6.593 (s, 1H), 5.433 (d, 1H, J=5.4 Hz), 5.064 (d, 1H, J=1.5 Hz), 4.435-4.26 (m, 3H), 3.53-3.38 (m, 6H), 2.60-2.16 (m, 6H), 2.045 (d, 6H, J=5.4 Hz), 1.369 (s, 3H),

MS (M+1): 573.2.

### Example 40

### Preparation of Hexanoic Acid 5-(9-amino-7-oxo-6,7-dihydro-2,3,5,6-tetraaza-benzo[cd]azulen-2-yl)-4-hexanoyloxy-2-hydroxymethyl-4-methyl-tetrahydro-furan-3-yl Ester (Compound 143)

### Step 1: 2-(5-O-tert-Butyldimethoxysilyl-2,3-bis-O-hexanoyl-2-C-methyl-β-D-ribofuanosyl)-2,6-dihydro-7H-2,3,5,6-tetraazabenzo[cd]azulen-7-one

Compound from Example 10, Step 1 (407 mg, 0.88 mmol) was added into a pre-stirred mixture of DCC (0.72 g, 3.5 mmol), DMAP (110 mg, 0.88 mmol) and hexanoic acid (0.45 mL, 3.5 mmol) in DMF (5 mL) over molecular sieves (4 Å). The resulting mixture was stirred at room temperature for 2 h and then concentrated under vacuum at 40° C. to a small volume. Solid material was filtered and filtrate evaporated. The evaporated residue was treated with MeOH, filtered and filtrate evaporated. The residue was purified by column chromatography on silica gel using 0-7% gradient of MeOH in CH₂Cl₂ to yield 471 mg of the target compound (81%).

MS: m/z=658.3

### Step 2: Hexanoic Acid 5-(9-amino-7-oxo-6,7-dihydro-2,3,5,6-tetraaza-benzo[cd]azulen-2-yl)-4-hexanoyloxy-2-hydroxymethyl-4-methyl-tetrahydro-furan-3-yl Ester (Compound 143)

To a solution of compound from Step 1 (385 mg, 0.59 mmol) in THF (4 mL) were added Et₃N (0.33 mL, 2.36 mmol) and Et₃N.3HF (0.19 mL, 1.18 mmol). The resulting mixture was stirred at room temperature for 2 h and then evaporated. The residue was purified by column chromatography on silica gel using 0-8% gradient of MeOH in CH₂Cl₂ to yield the target compound as a white solid (250 mg, 78%).

¹H NMR (DMSO-d₆): δ 10.84 (s, 1H), 8.32 (s, 1H), 8.03 (s, 1H), 6.76 (br s, 2H), 6.52 (s, 1H), 5.37 (d, 1H, J=3.2 Hz), 5.11 (m, 1H), 5.07 (d, 1H, J=1.0 Hz), 4.09 (dd, 1H, J=5.8 Hz and 2.6 Hz), 3.74 (m, 2H), 2.26-2.42 (m, 4H), 1.52 (m, 4H), 1.38 (s, 3H), 1.27 (m, 4H), 1.21 (m, 4H), 0.87 (t, 3H, J=6.7 Hz), 0.81 (t, 3H, J=6.9 Hz).

MS: m/z=544.2 (M+1).

### Example 41

### Preparation of 3-morpholin-4-yl-propionic acid 5-(9-amino-7-oxo-6,7-dihydro-2,3,5,6-tetraaza-benzo[cd]azulen-2-yl)-3,4-dihydroxy-4-methyl-tetrahydro-furan-2 ylmethyl Ester (Compound 144)

To a solution of 9-amino-2-(3,4-dihydroxy-5-hydroxymethyl-3-methyl-tetrahydro-furan-2-yl)-2,6-dihydro-2,3,5,6-tetraaza-benzo[cd]azulen-7-one (Compound 100, prepared according to WO 2006/093987, published on Sep. 8, 2006, 260 mg, 0.75 mmol) in 5.0 ml of DMF, were added 3-morpholin-4-yl-propionic acid hydrochloride (0.75 mmol, 146 mg), pyridine (59 μl, 0.75 mmol), DCC (156.0 mg, 0.75 mmol), and DMAP (0.07 mmol, 9.0 mg). After stirring for 2.5 h at room temperature, reaction mixture was filtered, and filtrate was concentrated in vacuo upto dryness. Residue was purified on ISCO combiflash using 40.0 g silica gel column with MeOH/CH₂Cl₂ (0 to 40% gradient for 30 min) as the eluents to yield 35.0 mg of the title compound.

¹H NMR (DMSO-d₆): δ 10.06 (s, 1H), 8.32 (s, 1H), 7.83 (s, 1H), 6.81 (bs, 2H), 6.21 (s, 1H), 5.53 (d, 1H, J=6.3), 5.4 (s, 1H), 5.05 (s, 1H), 4.5-3.89 (m, 4H), 3.58-3.17 (m, 8H), 2.44-2.36 (m, 4H), 0.79 (s, 3H)

MS (M+1): 489.2

### Example 42

### Preparation of Isobutyric Acid 2-{4-[2-(2-amino-3-methyl-butyrylamino)-acetoxy]-3-hydroxy-5-hydroxymethyl-3-methyl-tetrahydro-furan-2-yl}-7-oxo-6,7-dihydro-2H-2,3,5,6-tetraaza-benzo[cd]azulen-9-ylcarbamoyloxymethyl Ester (Compound 145)

### Step 1: (2-Benzyloxycarbonylamino-3-methyl-butyrylamino)-acetic acid 5-(9-amino-7-oxo-6,7-dihydro-2,3,5,6-tetraaza-benzo[cd]azulen-2-yl)-2-(tert-butyl-dimethyl-silanyloxymethyl)-4-hydroxy-4-methyl-tetrahydro-furan-3-yl Ester

To a solution of 9-amino-2-[5-(tert-butyl-dimethyl-silanyloxymethyl)-3,4-dihydroxy-3-methyl-tetrahydro-furan-2-yl]-2,6-dihydro-2,3,5,6-tetraaza-benzo[cd]azulen-7-one (Example 10, Step 1) (177 mg, 0.384 mmol) in 3.8 ml anhydrous DMF was added Cbz-Val-Gly-OH dipeptide (236 mg 0.767 mmol), DCC (158 mg, 0.767 mmol), and DMAP (9.7 mg, 0.080 mmol) and the mixture was stirred at room temperature overnight. The reaction was quenched with MeOH, concentrated in vacuo and purified on Isco CombiFlash purification system utilizing a 12 g silica gel column and 0-15% MeOH gradient in DCM as the eluent over 20 minutes to afford 175 mg (61%).

¹H NMR (DMSO-d₆): δ 10.11 (d, 1H, J=1.5 Hz), 8.42 (t, 1H, J=5.4 Hz), 8.33 (s, 1H), 7.86 (s, 1H), 7.33 (m, ), 6.81 (br s, 2H), 6.21 (s. 1H), 5.72 (s, 1H), 5.12 (d, 1H, J=8.4 Hz), 5.06 (d, 1H, J=1.8 Hz), 5.02 (d, 1H, J=1.5 Hz), 4.2-3.8 (m, ), 2.96 (m, 1H), 0.92-0.81 (m, )

MS: 572.3 (M+1)

### Step 2: Isobutyric Acid 2-[4-[2-(2-benzyloxycarbonylamino-3-methyl-butyrylamino)-acetoxy]-5-(tert-butyl-dimethyl-silanyloxymethyl)-3-hydroxy-3-methyl-tetrahydro-furan-2-yl]-7-oxo-6,7-dihydro-2H-2,3,5,6-tetraaza-benzo[cd]azulen-9-ylcarbamoyloxymethyl Ester

To a solution of the compound from step 1 (230 mg, 0.306 mmol) in pyridine 3 mL was added several molecular sieves and stirred at room temperature for 1 hour. To this solution was added TMSCl (38 uL, 0.306 mmol) and the reaction was stirred for an additional hour prior to cooling to 0° C. and adding isobutryloxymethyl carbonochloridate (product of Step 3, Example 13) (237 μL, 1.23 mmol) and the reaction was allowed to warm to room temperature. The reaction progress was monitored by QC-HPLC. The reaction was quenched with MeOH, concentrated in vacuo onto celite and purified on Isco CombiFlash purification system utilizing a 12 g silica gel column and 0-10% MeOH gradient in DCM as the eluent over 20 minutes to afford 140 mg of slightly impure material.

MS: 896.4 (M+1)

### Step 3: Isobutyric Acid 2-{4-[2-(2-benzyloxycarbonylamino-3-methyl-butyrylamino)-acetoxy]-3-hydroxy-5-hydroxymethyl-3-methyl-tetrahydro-furan-2-yl}-7-oxo-6,7-dihydro-2H-2,3,5,6-tetraaza-benzo[cd]azulen-9-ylcarbamoyloxymethyl Ester

To a solution of the product from Step 2 (140 mg, 0.156 mmol) in THF (1.5 mL) was added TEA.3HF (50.8 μL, 0.312 mmol) at 0° C. and the reaction was allowed to warm to room temperature. The reaction progress was monitored by QC-LCMS. The crude was concentrated in vacuo and the product was purified on reverse phase HPLC (20-100% buffer B over 20 minutes at 20 mL/min flow rate—Buffer A=H2O; Buffer B=ACN) to afford 35 mg.

### Step 4: Isobutyric Acid 2-{4-[2-(2-amino-3-methyl-butyrylamino)-acetoxy]-3-hydroxy-5-hydroxymethyl-3-methyl-tetrahydro-furan-2-yl}-7-oxo-6,7-dihydro-2H-2,3,5,6-tetraaza-benzo[cd]azulen-9-ylcarbamoyloxymethyl Ester (Compound 145)

To a solution of the product from Step 3 (33 mg, 0.0423 mmol) in MeOH containing 1% AcOH was added Pd/C (15 mg, 10% Palladium by weight) and the mixture was maintained under a blanket of hydrogen via balloon (1 atmosphere). The reaction progress was monitored by QC-HPLC. The palladium was filtered off, the filtrate was concentrated in vacuo and purified on reverse phase HPLC (0-100% buffer B over 20 minutes at 20 mL/min flow rate—Buffer A=H2O w/0.1% TFA; Buffer B=ACN w/0.1% TFA to afford 13 mg of compound 145 as the TFA salt.

¹H NMR (DMSO-d₆): δ 10.78 (s, 1H), 9.84 (s, 1H), 8.81 (s, 1H), 8.37 (s, 1H), 8.08 (br s, 3H), 8.02 (s, 1H), 6.57 (d, 1H, J=1.8 Hz), 6.21 (s, 1H), 5.80 (s, 2H), 5.75 (br s, 1H), 5.20 (d, 1H, J=8.4 Hz), 5.05 (br s, 1H), 4.33-3.6 (m, 6H), 2.6 (m, 1H), 2.09 (m, 1H), 1.13 (s, 3H), 1.1 (s, 3H), 1.0-0.95 (m, 6H), 0.88 (s, 3H).

MS: 648.2 (M+1)

### Example 43

### Preparation of 2-Benzyloxycarbonylamino-3-methyl-butyric acid 2-(3-hydroxy-4-isobutyryloxy-5-isobutyryloxymethyl-3-methyl-tetrahydro-furan-2-yl)-7-oxo-6,7-dihydro-2H-2,3,5,6-tetraaza-benzo[cd]azulen-9-ylcarbamoyloxymethyl Ester (Compound 146)

The preparation of the title compound was described in Example 21, step 3.

### BIOLOGICAL AND PHARMOCOKINETIC EXAMPLES

### Biological and Pharmacokinetic Example 1

### Anti-Hepatitis C Activity

Compounds can exhibit anti-hepatitis C activity by inhibiting viral and host cell targets required in the replication cycle. A number of assays have been published to assess these activities. A general method that assesses the gross increase of HCV virus in culture is disclosed in U.S. Pat. No. 5,738,985 to Miles et al. In vitro assays have been reported in Ferrari et al J. of Vir., 73:1649-1654, 1999; Ishii et al., Hepatology, 29:1227-1235, 1999; Lohmann et al, J. of Bio. Chem., 274:10807-10815, 1999; and Yamashita et al., J. of Bio. Chem., 273:15479-15486, 1998.

### Replicon Assay

A cell line, ET (Huh-lucubineo-ET) was used for screening of compounds of the present invention for inhibition of HCV RNA dependent RNA polymerase. The ET cell line was stably transfected with RNA transcripts harboring a I₃₈₉luc-ubi-neo/NS3-3′/ET; replicon with firefly luciferase-ubiquitin-neomycin phosphotransferase fusion protein and EMCV-IRES driven NS3-5B polyprotein containing the cell culture adaptive mutations (E1202G; T1280I; K1846T) (Krieger at al, 2001 and unpublished). The ET cells were grown in DMEM, supplemented with 10% fetal calf serum, 2 mM Glutamine, Penicillin (100 IU/mL)/Streptomycin (100 μg/mL), 1× nonessential amino acids, and 250 μg/mL G418 (“Geneticin”). They were all available through Life Technologies (Bethesda, Md.). The cells were plated at 0.5-1.0×10⁴ cells/well in the 96 well plates and incubated for 24 hrs before adding the test compounds. The compounds were then added to the cells to achieve a final concentration of 5 or 50 μM. Luciferase activity was measured 48-72 hours later by adding a lysis buffer and the substrate (Catalog number Glo-lysis buffer E2661 and Bright-Glo luciferase system E2620 Promega, Madison, Wis.). Cells should not be too confluent during the assay. Percent inhibition of replication was plotted relative to no compound control. Under the same condition, cytotoxicity of the compounds was determined using cell proliferation reagent, WST-1 (Roche, Germany). The compounds showing antiviral activities, but no significant cytotoxicities were chosen to determine the EC₅₀ and TC₅₀, the effective concentration and toxic concentration at which 50% of the maximum inhibition is observed. For these determinations, 6 dilutions of each compound were used. Compounds were typically diluted 3 fold to span a concentration range of 250 fold. EC₅₀ and similarly TC₅₀ values were calculated by fitting % inhibition at each concentration to the following equation:

% inhibition=100%/[(EC₅₀/[I])^{b}+1]

where b is Hill's coefficient.

In some embodiments, the compounds of Formula (I) or the pharmaceutically acceptable salts or solvates thereof are also prodrugs of compound 100, the compound of Formula (I) wherein R, W, W¹, and W² are H. The compounds tested in the examples below were found to exhibit desirable bioavailability, solubility, and/or acid stability properties as prodrugs of compound 100.

### Biological and Pharmacokinetic Example 2

### In vivo Dosing

Prior to dosing, male beagle dogs were fasted overnight. Unless otherwise noted, prodrugs without a nitrogen protecting group were given two 10 mg tablets of famotidine 1 hour prior to dosing to normalize stomach pH. Prodrugs were dosed at 2 to 4 mg equivalents of compound 100 per kg of body weight to normal or portal vein cannulated male beagle dogs. Prodrugs were administered as aqueous/organic solutions containing propylene glycol, polyethylene glycol, ethanol, di-methylsulfoxide, HCL and/or phosphate, unless specified otherwise in tables. Formulations for prodrugs without a nitrogen protecting group were buffered at neutral pH to maintain stability while nitrogen protected prodrugs were maintained at acidic pH. Blood samples were collected into tubes containing EDTA-K3 as an anticoagulant up to 24 hours post-dosing. The blood samples were centrifuged at 4° C. to separate plasma. Plasma was prepared by protein precipitation by adding acetonitrile to a final concentration of 60% in the presence of internal standard. Samples (200 μL) were dried down completely for approximately 30 minutes and reconstituted with 60 μL 20% acetonitrile.

Parent nucleoside levels in plasma samples were analyzed by reversed phase liquid chromatography coupled to a triple quadrupole mass spectrometer running in positive multiple reaction monitoring mode. For example, some samples were analyzed using an Aquity HPLC BEH C18 1.7 um 2.1×50 mm column and a mobile phase A containing 0.2% formic acid in 1% acetonitrile/water and mobile phase B containing 0.2% formic acid in 95% acetonitrile/water. The following elution program was applied using a binary pump system:

TABLE 2

| | Flow Rate | | |
|---|---|---|---|
| Time (min) | (mL/min) | Mobile Phase A (%) | Mobile Phase B (%) |
| 0.00 | 0.650 | 99.0 | 1.0 |
| 4.00 | 0.650 | 25.0 | 75.0 |
| 4.40 | 0.650 | 15.0 | 85.0 |
| 4.50 | 0.650 | 99.0 | 1.0 |
| 5.00 | 0.650 | 99.0 | 1.0 |

Levels of the parent nucleoside were quantitated by comparing peak area to that of a seven point standard curve made with authentic stock solutions. Separately prepared low and high quality control standards were analyzed in each analytical run to assure acceptable accuracy and precision. The results are summarized in the following tables.

TABLE 3
Maximum plasma concentration and plasma exposure to
parent nucleoside compound 100 upon administration
of ester prodrugs to famotidine pretreated dogs.

| | Cₘₐₓ/D | AUC_{0-∞}/D |
|---|---|---|
| Compound | (nM/(mg- | (nM · hr/(mg- |
| No. | eqv/kg)) | eqv/kg)) |
| 100 | 38 | 87 |
| 114 | 34 | 102 |
| 115 | 25 | 78 |
| 107 | 32 | 111 |
| 105 | 28 | 55 |
| 135 | 8 | 26 |
| 134 | 39 | 80 |
| 117 | 80 | 220 |
| 127 | 110 | 231 |
| 132 | 25 | 91 |
| 106 | 89 | 150 |
| 136 | 112 | 280 |
| 139 | 63 | 209 |
| 143 | 81 | 206 |
| 122 | 18 | 91 |

Cₘₐₓ/D is the maximum plasma concentration of the parent nucleoside compound 100 divided by the mg-equivalents of compound 100 per kg of body weight dosed.
AUC_{0-∞}/D is the exposure extrapolated to infinity of the parent nucleoside compound 100 divided by the mg-equivalents of compound 100 per kg of body weight dosed.
Values represent the mean of results from 3 dogs.

TABLE 4
Maximum plasma concentration and plasma exposure
to parent nucleoside compound 100 upon administration
of acid stable nitrogen prodrugs to dogs.

| | Cₘₐₓ/D | AUC_{0-∞}/D |
|---|---|---|
| Compound | (nM/(mg- | (nM · hr/(mg- |
| No. | eqv/kg)) | eqv/kg)) |
| 120 | 15 | 43 |
| 111 | 20 | 75 |
| 109 | 42 | 95 |
| 112 | 8 | ND |
| 110 | 28 | 90 |
| 118 | 29 | 85 |
| 128 | 35 | 111 |
| 119 | 15 | 71 |
| 123 | 25 | 134 |

Cₘₐₓ/D is the maximum plasma concentration of the parent nucleoside compound 100 divided by the mg-equivalents of compound 100 per kg of body weight dosed.
AUC_{0-∞}/D is the exposure extrapolated to infinity of the parent nucleoside compound 100 divided by the mg-equivalents of compound 100 per kg of body weight dosed.
Values represent the mean of results from 3 dogs.

TABLE 5
Formulation dependence of the maximum plasma concentration and plasma exposure
to parent nucleoside compound 100 upon administration of prodrugs to dogs.

| | | Cₘₐₓ/D | AUC_{0-∞}/D |
|---|---|---|---|
| Compound | | (nM/(mg- | (nM · hr/(mg- |
| No. | Formulation | eqv/kg)) | eqv/kg)) |
| 134 | 0.7 mg/mL (4 mL/kg), 2.8% DMSO, 30% | 39 | 80 |
| | PG, 67.2% phosphate buffer pH 7.7 | | |
| | Average dose 2.73 mg/kg powder in | 19 | 87 |
| | capsule (65%, mean 29.52 mg/capsule), | | |
| | 35% pregelatinized starch | | |
| | 0.7 mg/mL (4 mL/kg), 5% EtOH, 5% | 140 | 320 |
| | Solutol HS-15, 45% PEG, 45% water (pH | | |
| | 7, 0.01 M phosphate) | | |
| | 1.4 mg/mL (4 mL/kg), 4% DMSO, 40% | 99 | 278 |
| | PG, 56% phosphate buffer pH 7.4 | | |
| 128 | 0.91 mg/mL (4 mL/kg), 5% ethanol, 5% | 35 | 111 |
| | DMSO, 30% water, 60% PEG 400 (pH 3, | | |
| | HCl) | | |
| | 0.91 mg/mL (4 mL/kg), 5.0% Ethanol, 5% | 236 | 505 |
| | Solutol HS-15, 45% PEG, 45% Water (pH | | |
| | 3.0, 0.01 M sodium Phosphate) | | |
| 117 | 0.68 mg/mL (4 mL/Kg), 2.7 DMSO, 30% | 80 | 220 |
| | PG, 67% phosphate buffer pH 7 | | |
| | 3.42 mg/mL (2 mL/Kg), 4% EtOH, 4% | 59 | 314 |
| | Labrasol, 4% Solutol HS 15, 8% PG, 30% | | |
| | PEG 400, 50% water (buffered with 0.2M | | |
| | tri-sodium citrate, pH 7) | | |
| | 0.68 mg/mL (4 mL/Kg), 2.7% EtOH, 2.3% | 76 | 220 |
| | DMSO, 5% Solutol, 45% PEG, 45% water | | |
| | pH 7 (0.1M phosphate) | | |
| | 18.8 mg/mL (4 mL/Kg), 4% EtOH, 4% | 62 | 185 |
| | Labrasol, 4% Solutol HS 15, 8% PG, 30% | | |
| | PEG 400, 50% water (0.2M tri-sodium | | |
| | citrate, pH 7) | | |
| | Average dose 6.77 mg/kg powder in | <4 | <51 |
| | capsule (50%, mean 75.2 mg/capsule), 50% | | |
| | lactose | | |
| | Average dose 6.91 mg/kg powder in | <17 | <90 |
| | capsule (50%, mean 75.4 mg/capsule), 43% | | |
| | lactose, 7% sodium lauryl sulfate | | |
| | Average dose 2.80 mg/kg powder in | 10 | 80 |
| | capsule (50% mean 29.15 mg/capsule), | | |
| | 50% pregelatanized starch | | |
| | 0.68 mg/mL (4 mL/Kg), 2.7% DMSO, 30% | 10 | 75 |
| | PG, 67.3% water pH 7 (0.1M phosphate) | | |
| | 0.68 mg/mL (4 mL/Kg), 2.7% DMSO, 30% | ND | ND |
| | PG, 67.3% water pH 7 (0.01M phosphate) | | |
| | 0.68 mg/mL (4 mL/Kg), 2.5% DMSO, 5% | 96 | 295 |
| | Solutol HS 15, 30% PEG 400, 62.5% water | | |
| | pH 7 (0.2M tri-sodium citrate) | | |
| | 0.68 mg/mL (4 mL/Kg), 1% EtOH, 4% | 47 | 163 |
| | Solutol HS 15, 4% Labrasol, 46% PG, 45% | | |
| | water pH 7.5 (0.25M tri-sodium citrate) | | |
| | 0.68 mg/mL (4 mL/Kg), 1% EtOH, 4% | 97 | 339 |
| | Solutol HS 15, 4% Labrasol, 46% PG, 45% | | |
| | water pH 7.5 (0.25M tri-sodium citrate) | | |

Cmax/D is the maximum plasma concentration of the compound 100 divided by the mg-equivalents of compound 100 per kg of body weight dosed.
AUC0-∞/D is the exposure extrapolated to infinity of compound 100 divided by the mg-equivalents of compound 100 per kg of body weight dosed.
Values represent the mean of results from 3 dogs.
20 mg of famotidine was given 1 hr prior to dosing with compound 134 or 117 to increase stomach pH unless otherwise indicated.

### Biological and Pharmacokinetic Example 3

### Solubility

The solubility for certain compounds were determined using the following protocol and procedure. The results are summarized in Table 6.

### Protocol for Solubility:

1) The solution, sterile water or phosphate buffer solution (PBS), was added to the test compound tube to make the final concentration 10 mg/mL.

2) The sample tube was vortexed and incubated at 37° C. for 24 hours. During the incubation period, the sample tube was vortexed several times.

3) After the incubation, vortex the tube and centrifuge the tube at 13,000 rpm for 10 mins using an Eppendorf Centrifuge Model 5415C. If the solution was still cloudy, centrifuge it for longer until a clear supernatant was achieved.

4) The supernatant was diluted to 1×, 10× and 100× in 50% ACN in water.

5) A six point standard curve was prepared separately to make the final concentrations of 1 μg/mL, 5 μg/mL, 10 μg/mL, 20 μg/mL, 40 μg/mL and 60 μg/mL.

6) Samples were quantified using an HPLC with UV detector.

7) From the three concentrations of 1×, 10× and 100× supernatant, choose the value, which was fallen into the standard curve range (1 ug/mL-60 ug/mL), as the final result. If all the results were out of the curve range, adjust using a dilution factor to make the value within the curve range.

TABLE 6

| | | Acid | |
|---|---|---|---|
| | Solubility | Stability | |
| Compound | (Shaking | (T ½, mins | |
| No. | Flask) | @ pH 4.5) | |
| 100 | 0.011 mg/mL PBS | <15 | min |
| 101 | 0.137 mg/mL PBS | <30 | min |
| 102 | 0.011 mg/mL PBS | | |
| 103 | | ≦30 | min |
| 105 | 9.065 mg/mL PBS | <30 | min |
| 106 | 0.065 mg/mL PBS | <30 | min |
| 107 | 9.147 mg/mL PBS | | |
| 108 | 0.028 mg/mL PBS | <15 | min |
| 109 | 0.215 mg/mL PBS | >2 | hours |
| 110 | 8.16 mg/mL PBS | | |
| 111 | 0.002 mg/mL PBS | | |
| 112 | 0.08 mg/mL PBS | | |
| 113 | 0.067 mg/mL PBS | <30 | min |
| 114 | 2.950 mg/mL PBS | | |
| 115 | 0.134 mg/mL PBS | <30 | min |
| 116 | 0.202 mg/mL PBS | <30 | min |
| 117 | 0.51 mg/mL PBS | | |
| 118 | 0.06 mg/mL PBS | | |
| 119 | 0.006 mg/mL PBS | | |
| 120 | 0.06 mg/mL PBS | | |
| 124 | 6.25 mg/mL PBS | <30 | min |
| 125 | 0.051 mg/mL PBS | | |
| 126 | 0.04 mg/mL PBS | | |
| 127 | 0.133 mg/mL PBS | | |
| 129 | 0.12 mg/mL (water) | >2 | hours |
| 130 | 0.85 mg/mL PBS | | |
| 131 | 8.06 mg/mL PBS | | |
| 132 | 0.044 mg/mL PBS | | |
| 133 | 0.017 mg/mL PBS | | |
| 134 | .067 mg/mL PBS | <30 | min |
| 135 | 7.276 mg/mL PBS | <15 | min |
| 138 | 0.032 mg/mL PBS | >2 | hours |

### Biological and Pharmacokinetic Example 4

### Chemical Stability in Acidic Solution

The chemical stability in acidic solution (acid stability) for certain compounds were determined using the following protocol and procedure. The results are summarized in Table 6 above.

### Protocol for Chemical Stability:

1) Prepare 25 μg/mL stock solution of the testing compound with 1:1=ACN:H₂O

2) Add 960 μL of pH 4.5 chemical solution to an incubation tube

3) Pre incubate the chemical solution tube for 5 min at 37° C.

4) Spike 40 μL stock solution to the pre-incubated solution to make the final concentration of 1 g/mL and incubate at 37° C.

5) Aliquot 100 μL of the sample at each time point. Add 100 μL of ACN and 10 μL internal standard to the sample.

6) Vortex and quantify the sample on LC/MS.

### Biological and Pharmacokinetic Example 5

### Permeability

Caco-2 cells were maintained in Dulbecco's Modification of Eagle's Medium (DMEM) with sodium pyruvate, Glutmax supplemented with 1% Pen/Strep, 1% NEAA and 10% fetal bovine serum in an incubator set at 37° C., 90% humidity and 5% CO₂. Caco-2 cells between passage 43 and 61 were grown to confluence over at least 21-days on 24 well PET (polyethylene-terephthalate) plates (BD Biosciences). Experiments were run using a new HBSS donor buffer from Invitrogen containing additional 10 mM HEPES, 15 mM Glucose with pH adjusted to pH 6.5. The receiver well used HBSS buffer supplemented with 1% BSA and the pH adjusted to pH 7.4. After an initial equilibration with transport buffer, TEER values were read to test membrane integrity. The experiment was started by the addition of buffers containing test compounds and 100 μl of solution is taken at 1 and 2 hrs from the receiver compartment. Removed buffer was replaced with fresh buffer and a correction was applied to all calculations for the removed material. Each compound was tested in 2 separate replicate wells for each condition. All samples were immediately collected into 400 μl 100% acetonitrile acid to precipitate protein and stabilize test compounds. Cells were dosed on the apical or basolateral side to determine forward (A to B) and reverse (B to A) permeability. Permeability through a cell free trans-well was also determined as a measure of cellular permeability through the membrane and non-specific binding. To test for non-specific binding and compound instability the total amount of drug was quantitated at the end of the experiment and compared to the material present in the original dosing solution as a percent recovery. Samples were analyzed by LC/MS/MS.

The apparent permeability, Pₐₚₚ, and % recovery were calculated as follows:

Pₐₚₚ=(dR/dt)×Vᵣ/(A×D₀)

% Recovery=100×((Vᵣ×R₁₂₀)+(V_{d}×D₁₂₀))/(V_{d}×D₀)

where,
dR/dt is the slope of the cumulative concentration in the receiver compartment versus time in μM/s based on receiver concentrations measured at 60 and 120 minute.
Vᵣ and V_{d} is the volume in the receiver and donor compartment in cm³, respectively.
A is the area of the cell monolayer (0.33 cm²).
D₀ and D₁₂₀ is the measured donor concentration at the beginning and end of the experiment, respectively.
R₁₂₀ is the receiver concentration at the end of the experiment (120 minutes).

The apparent permeability for certain compounds were determined using the above procedure. The data ranges are classified in Table 7. The results are summarized in Table 8.

TABLE 7
Data Range Classification:

| | |
|---|---|
| Pₐₚₚ (A to B) ≧ 1.0 × 10⁻⁶ cm/s | High |
| 1.0 × 10⁻⁶ cm/s > Pₐₚₚ (A to B) ≧ 0.5 × 10⁻⁶ cm/s | Medium |
| Pₐₚₚ (A to B) < 0.5 × 10⁻⁶ cm/s | Low |

TABLE 8
Permeability

| | Pₐₚₚ (A to B) × 10⁻⁶ cm/s |
|---|---|
| Compound No. | (Caco-2 A-B) |
| 100 | low |
| 101 | high |
| 138 | medium |
| 134 | low |
| 106 | high |
| 114 | medium |
| 135 | medium |
| 103 | low |
| 113 | medium |
| 116 | low |
| 102 | high |
| 105 | high |
| 107 | low |
| 115 | high |
| 124 | low |
| 108 | medium |
| 109 | low |
| 117 | High |

### FORMULATION EXAMPLES

The following are representative pharmaceutical formulations containing a compound of Formula (I).

### Formulation Example 1

### Tablet formulation

The following ingredients are mixed intimately and pressed into single scored tablets.

| | Quantity per |
|---|---|
| Ingredient | tablet, mg |
| compound | 400 |
| cornstarch | 50 |
| croscarmellose sodium | 25 |
| lactose | 120 |
| magnesium stearate | 5 |

### Formulation Example 2

### Capsule Formulation

The following ingredients are mixed intimately and loaded into a hard-shell gelatin capsule.

| | Quantity per |
|---|---|
| Ingredient | capsule, mg |
| compound | 200 |
| lactose, spray-dried | 148 |
| magnesium stearate | 2 |

### Formulation Example 3

### Suspension Formulation

The following ingredients are mixed to form a suspension for oral administration.

| Ingredient | Amount | |
|---|---|---|
| compound | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| colorings | 0.5 | mg |

| | |
|---|---|
| distilled water | q.s. (quantity |
| | sufficient) to |
| | 100 mL |

### Formulation Example 4

### Injectable Formulation

The following ingredients are mixed to form an injectable formulation.

| Ingredient | Amount |
|---|---|
| compound | 0.2 mg-20 mg |
| sodium acetate buffer solution, 0.4 M | 2.0 mL |
| HCl (1N) or NaOH (1N) | q.s. to suitable pH |
| water (distilled, sterile) | q.s. to 20 mL |

### Formulation Example 5

### Suppository Formulation

A suppository of total weight 2.5 g is prepared by mixing the compounds with Witepsol® H-15 (triglycerides of saturated vegetable fatty acid; Riches-Nelson, Inc., New York), and has the following composition:

| Ingredient | Amount |
|---|---|
| compound | 500 mg |
| Witepsol ® H-15 | balance |

What is claimed is:

1. A compound of Formula (I) 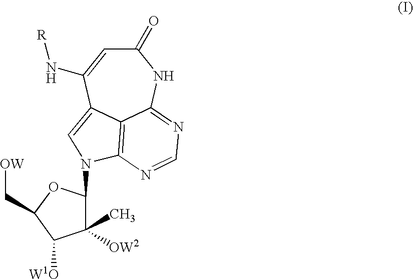
or a pharmaceutically acceptable salt or solvate thereof, wherein
R is selected from the group consisting of H and R¹(CO);
R¹ is selected from the group consisting of C₁₋₆ alkoxy, phenyl(C₁₋₆ alkoxy), substituted phenyl(C₁₋₆ alkoxy), (C₁₋₆ alkyl)(CO)O(C₁₋₆ alkoxy), substituted (C₁₋₆ alkyl)(CO)O(C₁₋₆ alkoxy), heterocyclyl(C₁₋₆ alkoxy), substituted heterocyclyl(C₁₋₆ alkoxy), amino(C₁₋₆ alkyl), substituted amino(C₁₋₆ alkyl), and acylamino(C₁₋₆ alkyl);
W and W¹ are independently selected from the group consisting of H, C₁₋₆ alkyl(CO), amino(C₁₋₆ alkyl)(CO), substituted amino(C₁₋₆ alkyl)(CO), acylamino(C₁₋₆ alkyl)(CO), heterocyclyl(C₁₋₆ alkyl)(CO), substituted heterocyclyl(C₁₋₆ alkyl)(CO), (C₁₋₆ alkyl)(CO)O(C₁₋₆ alkoxy), and substituted (C₁₋₆ alkyl)(CO)O(C₁₋₆ alkoxy);
W² is selected from the group consisting of H and C₁₋₆ alkyl(CO), heterocyclyl(C₁₋₆ alkyl)(CO); or OW¹ and OW² and together form a —O(CO)O— group; and
provided that when W, W¹, and W² are H, then R is not H or CH₃(CO).

2. A compound of claim 1 of Formula (Ia) 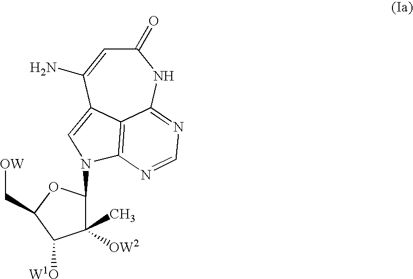
or a pharmaceutically acceptable salt or solvate thereof, wherein W, W¹, and W² are as defined in claim 1.

3. A compound of claim 1 of Formula (Ib) 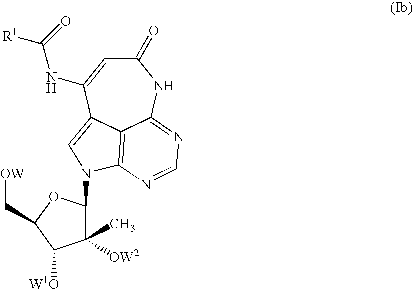
or a pharmaceutically acceptable salt or solvate thereof, wherein R¹ is selected from the group consisting of C₁₋₆ alkoxy, phenyl(C₁₋₆ alkoxy), substituted phenyl(C₁₋₆ alkoxy), (C₁₋₆ alkyl)(CO)O(C₁₋₆ alkoxy), substituted (C₁₋₆ alkyl)(CO)O(C₁₋₆ alkoxy), heterocyclyl(C₁₋₆ alkoxy), and substituted heterocyclyl(C₁₋₆ alkoxy); and W, W¹, and W² are as defined in Formula (I).

4. A compound of any one of claims 1 to 3 wherein at least one of W, W¹, or W² is C₁₋₆ alkyl(CO).

5. A compound of claim 4 wherein W and W¹ are independently C₁₋₆ alkyl(CO).

6. A compound of claim 5 wherein W, W¹, and W² are independently C₁₋₆ alkyl(CO).

7. A compound of claim 6 wherein W, W¹, and W² are independently selected from the group consisting of CH₃(CO), CH₃CH₂(CO), and (CH₃)₂CH(CO).

8. A compound of claim 7 wherein W, W¹, and W² are CH₃(CO).

9. A compound of claim 7 wherein W, W¹, and W² are CH₃CH₂(CO).

10. A compound of claim 7 wherein W, W¹, and W² are (CH₃)₂CH(CO).

11. A compound of claim 7 wherein W is H.

12. A compound of any one of claims 1 to 3 wherein W² is H.

13. A compound of any one of claims 1 to 3 wherein W¹ and W² are H.

14. A compound of any one of claims 1 to 3 wherein OW¹ and OW² together form a —O(CO)O— group.

15. A compound of claim 3 wherein R¹ is (C₁₋₆ alkyl)(CO)O(C₁₋₆ alkoxy).

16. A compound of claim 15 wherein R¹ is (CH₃)₂CH(CO)OCH₂O—.

17. A compound of claim 1 wherein R¹ is amino(C₁₋₆ alkyl).

18. A compound of claim 3 wherein R¹ is substituted heterocyclyl(C₁₋₆ alkoxy).

19. A compound of claim 3 wherein R¹ is amino(C₁₋₆ alkyl)(CO)O(C₁₋₆ alkoxy).

20. A compound of claim 3 wherein R¹ is substituted amino(C₁₋₆ alkyl)(CO)O(C₁₋₆ alkoxy).

21. A compound of claim 3 wherein R¹ is acylamino(C₁₋₆ alkyl)(CO)O(C₁₋₆ alkoxy).

22. A compound or a pharmaceutically acceptable salt or solvate thereof selected from the group consisting of:
Hexanoic acid 5-(9-amino-7-oxo-6,7-dihydro-2,3,5,6-tetraaza-benzo[cd]azulen-2-yl)-3,4-dihydroxy-4-methyl-tetrahydro-furan-2-ylmethyl ester (101);
Hexanoic acid 5-(9-amino-7-oxo-6,7-dihydro-2,3,5,6-tetraaza-benzo[cd]azulen-2-yl)-3-hexanoyloxy-4-hydroxy-4-methyl-tetrahydro-furan-2-ylmethyl ester (102);
Carbonic acid 5-(9-amino-7-oxo-6,7-dihydro-2,3,5,6-tetraaza-benzo[cd]azulen-2-yl)-3,4-dihydroxy-4-methyl-tetrahydro-furan-2-ylmethyl ester pentyl ester (103);
2-Amino-N-[2-(3,4-dihydroxy-5-hydroxymethyl-3-methyl-tetrahydro-furan-2-yl)-7-oxo-6,7-dihydro-2H-2,3,5,6-tetraaza-benzo[cd]azulen-9-yl]-acetamide (104);
Isobutyric acid 5-(9-amino-7-oxo-6,7-dihydro-2,3,5,6-tetraaza-benzo[cd]azulen-2-yl)-3,4-dihydroxy-4-methyl-tetrahydro-furan-2-ylmethyl ester (105);
Isobutyric acid 6-(9-amino-7-oxo-6,7-dihydro-2,3,5,6-tetraaza-benzo[cd]azulen-2-yl)-6a-methyl-2-oxo-tetrahydro-furo[3,4-d][1,3]dioxol-4-ylmethyl ester (106);
Acetic acid 5-(9-amino-7-oxo-6,7-dihydro-2,3,5,6-tetraaza-benzo[cd]azulen-2-yl)-3,4-dihydroxy-4-methyl-tetrahydro-furan-2-ylmethyl ester (107);
Acetic acid 6-(9-amino-7-oxo-6,7-dihydro-2,3,5,6-tetraaza-benzo[cd]azulen-2-yl)-6a-methyl-2-oxo-tetrahydro-furo[3,4-d][1,3]dioxol-4-ylmethyl ester (108);
Isobutyric acid 2-(3,4-dihydroxy-5-hydroxymethyl-3-methyl-tetrahydro-furan-2-yl)-7-oxo-6,7-dihydro-2H-2,3,5,6-tetraaza-benzo[cd]azulen-9-ylcarbamoyloxymethyl ester (109);
2-Amino-3-methyl-butyric acid 4-hydroxy-2-hydroxymethyl-5-(9-isobutyryloxymethoxycarbonylamino-7-oxo-6,7-dihydro-2,3,5,6-tetraaza-benzo[cd]azulen-2-yl)-4-methyl-tetrahydro-furan-3-yl ester (110);
Isobutyric acid 5-(9-acetoxymethoxycarbonylamino-7-oxo-6,7-dihydro-2,3,5,6-tetraaza-benzo[cd]azulen-2-yl)-4-hydroxy-2-isobutyryloxymethyl-4-methyl-tetrahydro-furan-3-yl ester (111);
Isobutyric acid 2-(4-acetoxy-3-hydroxy-5-hydroxymethyl-3-methyl-tetrahydro-furan-2-yl)-7-oxo-6,7-dihydro-2H-2,3,5,6-tetraaza-benzo[cd]azulen-9-ylcarbamoyloxymethyl ester (112);
Hexanoic acid 5-(9-amino-7-oxo-6,7-dihydro-2,3,5,6-tetraaza-benzo[cd]azulen-2-yl)-4-hydroxy-2-hydroxymethyl-4-methyl-tetrahydro-furan-3-yl ester (113);
Acetic acid 5-(9-amino-7-oxo-6,7-dihydro-2,3,5,6-tetraaza-benzo[cd]azulen-2-yl)-4-hydroxy-2-hydroxymethyl-4-methyl-tetrahydro-furan-3-yl ester (114);
Isobutyric acid 5-(9-amino-7-oxo-6,7-dihydro-2,3,5,6-tetraaza-benzo[cd]azulen-2-yl)-4-hydroxy-2-hydroxymethyl-4-methyl-tetrahydro-furan-3-yl ester (115);
9-Amino-2-(6-hydroxymethyl-3a-methyl-2-oxo-tetrahydro-furo[3,4-d][1,3]dioxol-4-yl)-2,6-dihydro-2,3,5,6-tetraaza-benzo[cd]azulen-7-one (116);
Acetic acid 4-acetoxy-2-acetoxymethyl-5-(9-amino-7-oxo-6,7-dihydro-2,3,5,6-tetraaza-benzo[cd]azulen-2-yl)-4-methyl-tetrahydro-furan-3-yl ester (117);
Isobutyric acid 2-(4-acetoxy-5-acetoxymethyl-3-hydroxy-3-methyl-tetrahydro-furan-2-yl)-7-oxo-6,7-dihydro-2H-2,3,5,6-tetraaza-benzo[cd]azulen-9-ylcarbamoyloxymethyl ester (118);
Isobutyric acid 4-hydroxy-3-isobutyryloxy-4-methyl-5-[9-(5-methyl-2-oxo-[1,3]dioxol-4-ylmethoxycarbonylamino)-7-oxo-6,7-dihydro-2,3,5,6-tetraaza-benzo[cd]azulen-2-yl]-tetrahydro-furan-2-ylmethyl ester (119);
Acetic acid 3-acetoxy-5-(9-acetoxymethoxycarbonylamino-7-oxo-6,7-dihydro-2,3,5,6-tetraaza-benzo[cd]azulen-2-yl)-4-hydroxy-4-methyl-tetrahydro-furan-2-ylmethyl ester (120);
2-Amino-3-methyl-butyric acid 2-(3-hydroxy-4-isobutyryloxy-5-isobutyryloxymethyl-3-methyl-tetrahydro-furan-2-yl)-7-oxo-6,7-dihydro-2H-2,3,5,6-tetraaza-benzo[cd]azulen-9-ylcarbamoyloxymethyl ester (121);
3-Morpholin-4-yl-propionic acid 5-(9-amino-7-oxo-6,7-dihydro-2,3,5,6-tetraaza-benzo[cd]azulen-2-yl)-4-hydroxy-4-methyl-3-(3-morpholin-4-yl-propionyloxy)-tetrahydro-furan-2-ylmethyl ester (122);
Isobutyric acid 2-(3,4-diacetoxy-5-acetoxymethyl-3-methyl-tetrahydro-furan-2-yl)-7-oxo-6,7-dihydro-2H-2,3,5,6-tetraaza-benzo[cd]azulen-9-ylcarbamoyloxymethyl ester (123);
2-Acetylamino-3-methyl-butyric acid 5-(9-amino-7-oxo-6,7-dihydro-2,3,5,6-tetraaza-benzo[cd]azulen-2-yl)-3,4-dihydroxy-4-methyl-tetrahydro-furan-2-ylmethyl ester (124);
Isobutyric acid 4-hydroxy-2-hydroxymethyl-5-(9-isobutyryloxymethoxycarbonylamino-7-oxo-6,7-dihydro-2,3,5,6-tetraaza-benzo[cd]azulen-2-yl)-4-methyl-tetrahydro-furan-3-yl ester (125);
[2-(3,4-Dihydroxy-5-hydroxymethyl-3-methyl-tetrahydro-furan-2-yl)-7-oxo-6,7-dihydro-2H-2,3,5,6-tetraaza-benzo[cd]azulen-9-yl]-carbamic acid 5-methyl-2-oxo-[1,3]dioxol-4-ylmethyl ester (126);
Propionic acid 5-(9-amino-7-oxo-6,7-dihydro-2,3,5,6-tetraaza-benzo[cd]azulen-2-yl)-4-methyl-3,4-bis-propionyloxy-tetrahydro-furan-2-ylmethyl ester (127);
Isobutyric acid 4-hydroxy-3-isobutyryloxy-5-(9-isobutyryloxymethoxycarbonylamino-7-oxo-6,7-dihydro-2,3,5,6-tetraaza-benzo[cd]azulen-2-yl)-4-methyl-tetrahydro-furan-2-ylmethyl ester (128);
Isobutyric acid 3,4-dihydroxy-5-(9-isobutyryloxymethoxycarbonylamino-7-oxo-6,7-dihydro-2,3,5,6-tetraaza-benzo[cd]azulen-2-yl)-4-methyl-tetrahydro-furan-2-ylmethyl ester (129);
Propionic acid 5-(9-amino-7-oxo-6,7-dihydro-2,3,5,6-tetraaza-benzo[cd]azulen-2-yl)-4-hydroxy-4-methyl-3-propionyloxy-tetrahydro-furan-2-ylmethyl ester (130);
Propionic acid 5-(9-amino-7-oxo-6,7-dihydro-2,3,5,6-tetraaza-benzo[cd]azulen-2-yl)-3,4-dihydroxy-4-methyl-tetrahydro-furan-2-ylmethyl ester (131);
Isobutyric acid 5-(9-amino-7-oxo-6,7-dihydro-2,3,5,6-tetraaza-benzo[cd]azulen-2-yl)-3,4-bis-isobutyryloxy-4-methyl-tetrahydro-furan-2-ylmethyl ester (132); Isobutyric acid 4-acetoxy-5-(9-amino-7-oxo-6,7-dihydro-2,3,5,6-tetraaza-benzo[cd]azulen-2-yl)-3-isobutyryloxy-4-methyl-tetrahydro-furan-2-ylmethyl ester (133);
Isobutyric acid 5-(9-amino-7-oxo-6,7-dihydro-2,3,5,6-tetraaza-benzo[cd]azulen-2-yl)-4-hydroxy-3-isobutyryloxy-4-methyl-tetrahydro-furan-2-ylmethyl ester (134);
Acetic acid 3-acetoxy-5-(9-amino-7-oxo-6,7-dihydro-2,3,5,6-tetraaza-benzo[cd]azulen-2-yl)-4-hydroxy-4-methyl-tetrahydro-furan-2-ylmethyl ester (135);
Isobutyric acid 4-acetoxy-5-acetoxymethyl-2-(9-amino-7-oxo-6,7-dihydro-2,3,5,6-tetraaza-benzo[cd]azulen-2-yl)-3-methyl-tetrahydro-furan-3-yl ester (136);
[2-(3,4-Dihydroxy-5-hydroxymethyl-3-methyl-tetrahydro-furan-2-yl)-7-oxo-6,7-dihydro-2H-2,3,5,6-tetraaza-benzo[cd]azulen-9-yl]-carbamic acid pentyl ester (138);
Isobutyric acid 5-(9-amino-7-oxo-6,7-dihydro-2,3,5,6-tetraaza-benzo[cd]azulen-2-yl)-2-hydroxymethyl-4-isobutyryloxy-4-methyl-tetrahydro-furan-3-yl ester (139);
3-Morpholin-4-yl-propionic acid 4-acetoxy-5-acetoxymethyl-2-(9-amino-7-oxo-6,7-dihydro-2,3,5,6-tetraaza-benzo[cd]azulen-2-yl)-3-methyl-tetrahydro-furan-3-yl ester (140);
Isobutyric acid 5-(9-benzyloxycarbonylamino-7-oxo-6,7-dihydro-2,3,5,6-tetraaza-benzo[cd]azulen-2-yl)-4-hydroxy-3-isobutyryloxy-4-methyl-tetrahydro-furan-2-ylmethyl ester (141);
3-Morpholin-4-yl-propionic acid 4-acetoxy-2-acetoxymethyl-5-(9-amino-7-oxo-6,7-dihydro-2,3,5,6-tetraaza-benzo[cd]azulen-2-yl)-4-methyl-tetrahydro-furan-3-yl ester (142);
Hexanoic acid 5-(9-amino-7-oxo-6,7-dihydro-2,3,5,6-tetraaza-benzo[cd]azulen-2-yl)-4-hexanoyloxy-2-hydroxymethyl-4-methyl-tetrahydro-furan-3-yl ester (143);
3-Morpholin-4-yl-propionic acid 5-(9-amino-7-oxo-6,7-dihydro-2,3,5,6-tetraaza-benzo[cd]azulen-2-yl)-3,4-dihydroxy-4-methyl-tetrahydro-furan-2-ylmethyl ester (144);
Isobutyric acid 2-{4-[2-(2-amino-3-methyl-butyrylamino)-acetoxy]-3-hydroxy-5-hydroxymethyl-3-methyl-tetrahydro-furan-2-yl}-7-oxo-6,7-dihydro-2H-2,3,5,6-tetraaza-benzo[cd]azulen-9-ylcarbamoyloxymethyl ester (145); and
2-Benzyloxycarbonylamino-3-methyl-butyric acid 2-(3-hydroxy-4-isobutyryloxy-5-isobutyryloxymethyl-3-methyl-tetrahydro-furan-2-yl)-7-oxo-6,7-dihydro-2H-2,3,5,6-tetraaza-benzo[cd]azulen-9-ylcarbamoyloxymethyl ester (146).

23. A pharmaceutical composition comprising a pharmaceutically acceptable carrier and a therapeutically effective amount of a compound of any one of claims 1, 2, 3, or 22.

24. A method for treating a viral infection in a patient mediated at least in part by a virus in the Flaviviridae family of viruses which method comprises administering to the patient a compound of any one of claims 1, 2, 3, or 22.

25. The method of claim 24 wherein said viral infection is a hepatitis C mediated viral infection.

26. A method for preparing a compound of Formula (II) or a pharmaceutically acceptable salt thereof 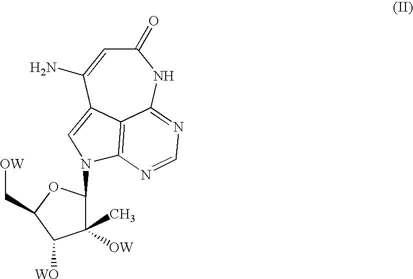
wherein W is optionally substituted C₁₋₆ alkyl(CO), said method comprising:
(a) reacting a compound of Formula (IIa) 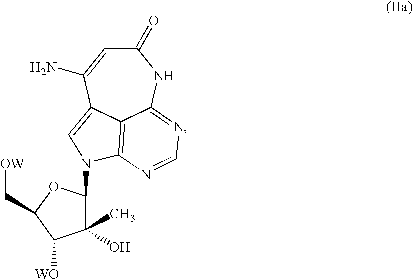
wherein W and W¹ are independently H or optionally substituted C₁₋₆ alkyl(CO), with optionally substituted C₁₋₆ alkyl(CO)OH and an amide coupling agent to form a compound of Formula (II); and
(b) optionally reacting a compound of Formula (II) with an acid to form a pharmaceutically acceptable salt thereof.
